(19) United States
(12) Patent Application Publication
(10) Pub. No.: US 2024/0228545 A9
(43) Pub. Date: Jul. 11, 2024

(54) CHARGE MODIFIED DESIGNED REPEAT DOMAINS AND THEIR USE

(71) Applicant: MOLECULAR PARTNERS AG, Zurich-Schlieren (CH)

(72) Inventors: Andreas BOSSHART, Zurich-Schlieren (CH); Daniel STEINER, Zurich-Schlieren (CH); Christian REICHEN, Zurich-Schlieren (CH)

(21) Appl. No.: 18/362,079

(22) Filed: Jul. 31, 2023

(30) Foreign Application Priority Data
Aug. 1, 2022 (EP) 22188160
Jan. 10, 2023 (EP) 23151023
Apr. 14, 2023 (EP) 23168056

(51) Int. Cl. C07K 14/00; A61K 47/42

(52) U.S. Cl. CPC C07K 14/00; A61K 47/42

(57) ABSTRACT

The present invention relates to designed ankyrin repeat domains having a reduced isoelectric point (pI) and/or having a reduced number of basic amino acid residues. The invention also provides such repeat domains linked to a drug moiety, for example a radionuclide or a cytotoxic agent. The invention furthermore provides methods for producing such repeat domains, as well as the use of such repeat domains in therapeutic and/or diagnostic methods. In addition, the invention provides recombinant proteins comprising such repeat domains, nucleic acids encoding such repeat domains or recombinant proteins, pharmaceutical compositions comprising such repeat domains, recombinant proteins nucleic acids, recombinant expression vectors and host cells, and the use of such proteins, nucleic acids or pharmaceutical compositions in methods for treating diseases, such as cancer.

### CROSS-REFERENCE TO RELATED APPLICATIONS

The present application claims the benefit of priority to EP22188160, filed on Aug. 1, 2022; EP23151023, filed on Jan. 10, 2023; and EP23168056, filed on Apr. 14, 2023. The disclosures of these patent applications are incorporated herein for all purposes by reference in their entirety.

### SEQUENCE LISTING

The XML-format Sequence Listing “SequenceListing.xml” submitted herewith, created on Jul. 31, 2023 with a size of 168,768 bytes, is hereby incorporated by reference in its entirety.

### FIELD OF THE DISCLOSURE

The present invention relates to designed ankyrin repeat domains having a reduced isoelectric point (pI) and/or having a reduced number of basic amino acid residues. The invention also provides such repeat domains linked to a drug moiety, for example a radionuclide or a cytotoxic agent. The invention furthermore provides methods for producing such repeat domains, as well as the use of such repeat domains in therapeutic and/or diagnostic methods. In addition, the invention provides recombinant proteins comprising such repeat domains, nucleic acids encoding such repeat domains or recombinant proteins, pharmaceutical compositions comprising such repeat domains, recombinant proteins nucleic acids, recombinant expression vectors and host cells, and the use of such proteins, nucleic acids or pharmaceutical compositions in methods for treating diseases, such as cancer.

### BACKGROUND

Targeted radiopharmaceuticals have emerged as a promising tool in the diagnosis and treatment of cancers. Such radiopharmaceuticals typically consist of a radioactive agent (e.g. radionuclide) linked to a binding molecule (e.g. antibodies or fragments thereof, engineered protein scaffolds, peptides or small molecules). By combining a specificity for a biological target with an ionizing radiation source, radiopharmaceuticals can concentrate radiation emission in the vicinity of a biomarker of interest. High target selectivity and tumor retention, low uptake in non-tumoral organs and tissues and a fast clearance are desired characteristics for precise cancer diagnostics (e.g. the radioactive agent reveals the tumor location) and/or therapy (e.g. the radioactive agent damages the tumor) with radiopharmaceuticals.

Early work in radioimmunotherapy highlighted important drawbacks of using radiolabeled antibodies, such as a slow extravasation and slow clearance of intact antibodies from the blood due to their size (150 kDa). With antibody based-radiopharmaceuticals, an optimal tumor-to-background ratio can typically be reached only after several days, therefore inducing indirect damage to radiosensitive organs and tissues such as bone marrow. Subsequently, alternative binders of lower molecular weights were developed to improve pharmacokinetics and increase tumor-to-normal tissue dose ratios. Drug clearance in the context of radiolabeled peptides, including small antibody fragments, is predominantly driven by renal excretion which involves the physiological processes of glomerular filtration, active tubular secretion, and tubular reabsorption. Glomerular filtration ensures that circulating cells and valuable macromolecular components of blood plasma are selectively retained based on molecular size. Molecules weighing more than 70 kDa or being larger than 4.2 nm in radius, and those bound to plasma proteins (such as albumin) undergo negligible glomerular filtration (Parihar, A. S. et al., Translational Oncology 15.1 (2022): 101295).

Several radiopharmaceuticals, due to their inherent properties, are retained within the kidneys, herewith contributing to an increased radiation absorbed dose to the kidneys. Even though small format binding molecules with a lower molecular weight can provide a combined advantage of rapid targeting and rapid clearance with minimal uptake in normal tissues or organs early after injection, their use also induces undesired high renal accumulation of radioactivity thereby hindering their broader clinical application. Both the choice of radionuclide and the nature of the binding molecule may impact the severity of nephrotoxicity resulting from such high radioactivity accumulation in kidneys (Chigoho, D. M. et al., Current opinion in chemical biology 63 (2021): 219-228).

A key contributor to nephrotoxicity is the process of renal reabsorption. Low to moderate molecular weight radiolabeled molecules are readily filtered through the glomerulus and are subsequently reabsorbed via a charge attraction to the negatively charged phospholipid bilayer of proximal tubular cells and subsequently catabolized in the cells. After proteolytic degradation in lysosomes, radiolabeled catabolites are released and, depending on their physical properties, are either freely washed out of the cells (non-residualizing radionuclide) or are retained intracellularly (residualizing radionuclide). Residualizing radionuclides are typically advantageous from the viewpoint of tumor cytotoxicity but can increase the toxicity profile due to off-target localization in normal tissues.

To palliate such renal retention, pharmacological and/or physicochemical approaches have been tried, for instance by co-injecting positively charged amino acids which inhibit the reabsorption of radiotracers from the negatively charged proximal tubular membrane and/or by modulating the net charge of the binding protein used in the conjugate through acylation, respectively. It was reported that the renal clearance and the tumor targeting of ⁹⁹ mTc-labeled humanized anti-Tac monoclonal antibody Fab could be enhanced by neutralizing the positive charges of the Fab through acylation. In addition, co-injection of lysine further improved blockage of renal uptake (Kim, M. K, et al., Nuclear medicine and biology, 29.2 (2002): 139-146). Further, the effect of single amino acid substitutions on renal radioactivity accumulation has been studied by Akizawa et al., in the context of a very short, 8 amino acid peptide (¹¹¹In-DTPA-conjugated octreotide derivatives), but not for larger binding domains or proteins (Akizawa, H., et al., Nuclear medicine and biology 28.7 (2001): 761-768).

Pharmacological approaches to lower the uptake of peptide- and protein-based radiopharmaceuticals in the kidneys may help to circumvent the efforts required in modulating the structural properties of radiolabeled compounds. However, administration of such mitigation compounds acting on various parts of the reabsorption system in the kidney has shown not to be effective for all radiopharmaceuticals. A study on the prevention of renal uptake of ⁹⁹ mTc-labeled designed ankyrin repeat proteins (DARPins) showed that common clinical strategies were not effective for reducing kidney uptake of these radiolabeled DARPins in mice. More specifically, co-injection of lysine or Gelofusine did not reduce renal uptake. Pre-administration of high doses of maleate or fructose, which inhibit ATP-mediated endocytosis, resulted in a reduction of kidney uptake of these protein scaffolds, but at a required dose not suitable for a clinical application. No other compounds were effective. As such, according to the authors, the study suggests that the renal uptake of ⁹⁹ mTc-labeled DARPins proceeds through a mechanism independent of DARPin structure and binding site composition. (Altai, M. et al., EJNMMI research 10.1 (2020): 1-8). Thus, despite attempts to reduce renal uptake and mitigate nephrotoxicity with pharmacological and/or physicochemical approaches, this could not consistently be achieved and nephrotoxicity remains a hurdle for the application of radiopharmaceuticals in radiopharmaceutical therapy or diagnostics. Similar considerations apply to pharmaceuticals in which a small format target-specific binder is linked to a cytotoxic agent instead of a radionuclide, such as, for example, in protein conjugates using antibody mimetics or small binding moieties (Richards, D. A. Drug Discovery Today: Technologies 30 (2018): 35-46).

Taken together, there remains a need for improved target-specific binders with beneficial properties that result, when administered to a mammal, in reduced renal accumulation, in particular in reduced renal accumulation of a linked drug moiety, such as a radionuclide or a cytotoxic agent. Such improved target-specific binders may be used in therapeutic or diagnostic applications, such as, for example, in radiopharmaceuticals or cytotoxic drug-conjugates.

### SUMMARY

The present invention provides engineered DARPins which have a reduced number of basic amino acids (positive charges) and/or a reduced isoelectric point (pI) compared to conventional DARPins. These properties have been surprisingly found to reduce renal accumulation of a linked drug moiety (such as, e.g., a radionuclide) following in vivo administration of drug moiety-linked DARPins of the invention.

DARPins are small engineered scaffold proteins (about 14 kDa for a single designed repeat domain) that can be selected to bind a given target protein with high affinity and specificity. Their application in the context of radiopharmaceutical therapy or diagnostics has started to be investigated, for example in a phase I clinical trial involving ⁹⁹ mTc labeled DARPin for breast cancer imaging (Bragina, O, et al., Journal of Nuclear Medicine 63.4 (2022): 528-535). However, as with other radiopharmaceuticals based on low molecular weight binders, solutions to address nephrotoxicity are required to fully exploit the potential of DARPin-based radiopharmaceuticals. Modification of the net charge of DARPins has been described, e.g. in WO2016023898 (in this case DARPins were modified to become less negative, in an approach to create DARPins capable of crossing the lipophilic membrane barrier of cells), but modification of the charge properties or of the pI of a DARPin has not been assessed in the context of renal accumulation.

The present invention relates to designed ankyrin repeat proteins (DARPins) having structural modifications that improve their pharmacokinetic properties, in particular leading to reduced renal accumulation. More particularly, the invention provides designed ankyrin repeat domains with a reduced number of basic amino acid residues and/or a reduced isoelectric point (pI), as compared to commonly described designed ankyrin repeat domains. The invention also provides such improved repeat domains linked to a drug moiety, for example a radionuclide or a cytotoxic agent. The invention furthermore provides methods for producing such repeat domains, as well as the use of such repeat domains in therapeutic and/or diagnostic methods. In addition, the invention provides recombinant proteins comprising such repeat domains, nucleic acids encoding such repeat domains or recombinant proteins, pharmaceutical compositions comprising such repeat domains, recombinant proteins nucleic acids, recombinant expression vectors and host cells, and the use of such proteins, nucleic acids or pharmaceutical compositions in methods for treating diseases, such as cancer in a mammal, including a human.

The DARPins of the invention, when linked to a radioactive or cytotoxic agent, exhibit a reduced accumulation of the agent in the kidneys upon in vivo administration. Accordingly, the improved repeat domains and recombinant proteins provided in the present invention open the door to new therapeutic and diagnostic applications, for instance in the field of nuclear medicine such as in radiopharmaceutical therapy, or in cancer therapy using cytotoxic agent-conjugated proteins.

In one aspect, the invention provides a designed ankyrin repeat domain comprising an N-terminal capping module, at least one internal repeat module and a C-terminal capping module, wherein said repeat domain has at least one characteristic selected from the following list of characteristics:
(a) an isoelectric point (pI) equal to or lower than pH 4.07;
(b) among all amino acid residues comprised in said repeat domain, a percentage of basic amino acid residues equal to or lower than 7.0%; and
(c) among the framework residues comprised in said repeat domain, a percentage of basic amino acid residues equal to or lower than 6.1%.

In another aspect, the invention provides a method of generating a modified ankyrin repeat domain, the method comprising steps (a) and (b),
wherein step (a) is providing an ankyrin repeat domain which has an isoelectric point (pI) higher than pH 4.07, and/or among all amino acid residues comprised in the repeat domain, a percentage of basic amino acid residues higher than 7.0%, and/or among the framework residues comprised in the repeat domain, a percentage of basic amino acid residues higher than 6.1%; and wherein step (b) is (1) substituting at least one basic amino acid residue of the repeat domain of step (a) with a neutral or acidic amino acid residue; and/or
(2) substituting at least one neutral amino acid residue of the repeat domain of step (a) with an acidic amino acid;
   wherein said modified ankyrin repeat domain has at least one characteristic selected from the following list of characteristics:
(i) an isoelectric point (pI) equal to or lower than pH 4.07;
(ii) among all amino acid residues comprised in the repeat domain, a percentage of basic amino acid residues equal to or lower than 7.0%; and
(iii) among the framework residues comprised in the repeat domain, a percentage of basic amino acid residues equal to or lower than 6.1%.

In another aspect, the invention provides a designed ankyrin repeat domain comprising an N-terminal capping module, at least one internal repeat module and a C-terminal capping module, wherein said repeat domain is linked to a drug moiety, and wherein said repeat domain has at least one characteristic selected from the following list of characteristics:
(a) an isoelectric point (pI) equal to or lower than pH 4.6;
(b) among all amino acid residues comprised in said repeat domain, a percentage of basic amino acid residues equal to or lower than 12.0%;
(c) among all amino acid residues comprised in said repeat domain, a percentage of Arg and Lys residues equal to or lower than 8.0%;
(d) among the framework residues comprised in said repeat domain, a percentage of basic amino acid residues equal to or lower than 9.6%; and
(e) among the framework residues comprised in said repeat domain, a percentage of Arg and Lys residues equal to or lower than 6.7%.

In another aspect, the invention provides a method of generating a modified ankyrin repeat domain linked to a drug moiety, the method comprising steps (a) and (b):
wherein step (a) is providing an ankyrin repeat domain which has an isoelectric point (pI) higher than pH 4.6, and/or among all amino acid residues comprised in the repeat domain, a percentage of basic amino acid residues higher than 12.0%, and/or among all amino acid residues comprised in the repeat domain, a percentage of Arg and Lys residues higher than 8.0%, and/or among the framework residues comprised in the repeat domain, a percentage of basic amino acid residues higher than 9.6%, and/or among the framework residues comprised in the repeat domain, a percentage of Arg and Lys residues higher than 6.7%; and
wherein step (b) is (1) substituting at least one basic amino acid residue of the repeat domain of step (a) with a neutral or acidic amino acid residue; and/or
(2) substituting at least one neutral amino acid residue of the repeat domain of step (a) with an acidic amino acid;
   wherein said modified repeat domain has at least one characteristic selected from the following list of characteristics:
(i) an isoelectric point (pI) equal to or lower than pH 4.6;
(ii) among all amino acid residues comprised in the repeat domain, a percentage of basic amino acid residues equal to or lower than 12.0%;
(iii) among all amino acid residues comprised in the repeat domain, a percentage of Arg and Lys residues equal to or lower than 8.0%;
(iv) among the framework residues comprised in the repeat domain, a percentage of basic amino acid residues equal to or lower than 9.6%; and
(v) among the framework residues comprised in the repeat domain, a percentage of Arg and Lys residues equal to or lower than 6.7%.

In another aspect, the invention provides a designed ankyrin repeat domains obtainable or obtained by the method of the invention.

In another aspect, the invention provides recombinant proteins comprising such designed repeat domains of the invention.

In another aspect, the invention provides isolated nucleic acids encoding a designed repeat domain of the invention or encoding a recombinant protein of the invention, a recombinant expression vector comprising such nucleic acids, host cells comprising such expression vectors and pharmaceutical compositions comprising the designed repeat protein, recombinant protein, nucleic acid and/or recombinant expression vector of the invention and optionally at least one pharmaceutically acceptable carrier or diluent.

In another aspect, the invention provides a method of treating and/or diagnosing a medical condition, the method comprising the step of administering to a patient in need thereof a therapeutically and/or diagnostically effective amount of the designed repeat domain, recombinant protein, nucleic acid, or pharmaceutical composition of the invention. In one particular aspect, said medical condition is a cancer.

In another aspect, the invention provides the designed repeat domain, recombinant protein, nucleic acid, or pharmaceutical composition of the invention for use in a method of treating a medical condition. In one particular aspect, said medical condition is a cancer.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E):
E1. A designed ankyrin repeat domain comprising an N-terminal capping module, at least one internal repeat module and a C-terminal capping module, wherein said repeat domain has at least one characteristic selected from the following list of characteristics:
   (a) an isoelectric point (pI) equal to or lower than pH 4.07;
   (b) among all amino acid residues comprised in said repeat domain, a percentage of basic amino acid residues equal to or lower than 7.0%; and
   (c) among the framework residues comprised in said repeat domain, a percentage of basic amino acid residues equal to or lower than 6.1%.
E2. The repeat domain according to E1, wherein said repeat domain has at least two characteristics selected from said characteristics (a), (b) and (c).
E3. The repeat domain according to any one of E1 to E2, wherein said repeat domain has at least characteristics (a) and (b), (a) and (c), or (b) and (c).
E4. The repeat domain according to any one of E1 to E3, wherein said repeat domain has characteristics (a), (b) and (c).
E5. The repeat domain according to any one of E1 to E4, wherein said repeat domain binds specifically to a target.
E6. The repeat domain according to E5, wherein said repeat domain binds said target with a dissociation constant (K_{D}) lower than 10⁻⁷ M.
E7. The repeat domain according to any one of E1 to E6, wherein said repeat domain comprises one internal repeat module, two internal repeat modules, three internal repeat modules, or four internal repeat modules.
E8. The repeat domain according to any one of E1 to E7, wherein said repeat domain has a KR/DE ratio equal to or lower than 0.44, and/or wherein said repeat domain has a KR/DE ratio among the framework residues equal to or lower than 0.36, and/or wherein said repeat domain has a KR/DE ratio among all residues of the N-terminal capping module equal to or lower than 0.66.
E9. The repeat domain according to any one of E1 to E8, wherein each internal repeat module independently comprises a sequence selected from the group consisting of (1) SEQ ID NOs: 131 to 142 and (2) sequences in which up to 9 framework residues in any of SEQ ID NOs: 131 to 142 are substituted by another amino acid, and/or said N-terminal capping module comprises a sequence selected from the group consisting of (1) SEQ ID NOs: 25 and 115 to 122 and (2) sequences in which up to 9 framework residues in any of SEQ ID NOs: 25 and 115 to 122 are substituted by another amino acid, and/or said C-terminal capping module comprises a sequence selected from the group consisting of (1) SEQ ID NOs: 123 to 130 and (2) sequences in which up to 9 framework residues in any of SEQ ID NOs: 123 to 130 are substituted by another amino acid.
E10. The repeat domain according to any one of E1 to E9, wherein each internal repeat module independently comprises a sequence selected from the group consisting of (1) SEQ ID NO: 31 and (2) sequences in which up to 5 framework residues other than positions 1, 10, 13, 17, 19, 21, 22 and 26 in SEQ ID NO: 31 are substituted by another amino acid, and/or said N-terminal capping module comprises a sequence selected from the group consisting of (1) SEQ ID NO: 19 and (2) sequences in which up to 5 framework residues other than positions 5, 17, 20 and 23 in SEQ ID NOs: 19 are substituted by another amino acid, and/or said C-terminal capping module comprises a sequence selected from the group consisting of (1) SEQ ID NO: 40 and (2) sequences in which up to 5 framework residues other than positions 10, 11, 17, 18, 19, 22 and 26 in SEQ ID NO: 40 are substituted by another amino acid.
E11. The repeat domain according to any one of E1 to E10, wherein each internal repeat module independently comprises a sequence selected from SEQ ID NOs: 31 to 35, 61 to 62, 92 to 94 and 131 to 142, and/or said N-terminal capping module comprises a sequence selected from SEQ ID NOs: 19 to 25, 57 to 60, 65 to 81 and 115 to 122, and/or said C-terminal capping module comprises a sequence selected from SEQ ID NOs: 40 to 42, 63 to 64, 82 to 91 and 123 to 130.
E12. The repeat domain according to any one of E1 to E11, wherein each internal repeat module independently comprises a sequence selected from the group consisting of (1) SEQ ID NOs: 26 to 30 and 47 to 48 and (2) sequences in which up to 9 amino acids in any of SEQ ID NOs: 26 to 30 and 47 to 48 are substituted by another amino acid, and/or the N-terminal capping module comprises a sequence selected from the group consisting of (1) SEQ ID NOs: 15 to 18, 43 and 99 to 101 and (2) sequences in which up to 9 amino acids in any of SEQ ID NOs: 15 to 18, 43 and 99 to 101 are substituted by another amino acid, and/or the C-terminal capping module comprises a sequence selected from the group consisting of (1) SEQ ID NOs: 36 to 39, 54 and 102 to 103 and (2) sequences in which up to 9 amino acids in any of SEQ ID NOs: 36 to 39, 54 and 102 to 103 are substituted by another amino acid. In certain embodiments, the substitutions are substitutions according to Table 3. In further embodiments, the substitutions are conservative substitutions according to Table 3.
E12a. The repeat domain according to any one of E1 to E12, wherein each internal repeat module independently comprises a sequence selected from the group consisting of (1) SEQ ID NOs: 26 to 30 and 47 to 48 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 26 to 30 and 47 to 48 are substituted by another amino acid, and/or the N-terminal capping module comprises a sequence selected from the group consisting of (1) SEQ ID NOs: 15 to 18, 43 and 99 to 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 15 to 18, 43 and 99 to 101 are substituted by another amino acid, and/or the C-terminal capping module comprises a sequence selected from the group consisting of (1) SEQ ID NOs: 36 to 39, 54 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 36 to 39, 54 and 102 to 103 are substituted by another amino acid. In certain embodiments, the substitutions are substitutions according to Table 3. In further embodiments, the substitutions are conservative substitutions according to Table 3.
E12b. The repeat domain according to any one of E1 to E12a, wherein each internal repeat module independently comprises a sequence selected from the group consisting of (1) SEQ ID NOs: 26 to 30 and 47 to 48 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 26 to 30 and 47 to 48 are substituted by another amino acid.
E12c. The repeat domain according to any one of E1 to E12b, wherein the N-terminal capping module comprises a sequence selected from the group consisting of (1) SEQ ID NOs: 15 to 18, 43 and 99 to 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 15 to 18, 43 and 99 to 101 are substituted by another amino acid.
E12d. The repeat domain according to any one of E1 to E12c, wherein the C-terminal capping module comprises a sequence selected from the group consisting of (1) SEQ ID NOs: 36 to 39, 54 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 36 to 39, 54 and 102 to 103 are substituted by another amino acid.
E13. The repeat domain according to any one of E1 to E12, wherein the repeat domain comprises an amino acid sequence selected from the group consisting of (1) SEQ ID NOs: 104, 108, 109 and 112 to 114 and (2) sequences with at least 80% amino acid sequence identity among the framework residues of any one of SEQ ID NOs: 104, 108, 109 and 112 to 114.
E14. The repeat domain according to any one of E1 to E13, wherein the repeat domain comprises an amino acid sequence selected from the group consisting of (1) SEQ ID NOs: 8 to 9 and 95 to 98 and (2) sequences with at least 80% amino acid sequence identity with any one of SEQ ID NOs: 8 to 9 and 95 to 98.
E15. A method of generating a modified ankyrin repeat domain, the method comprising steps (a) and (b), wherein step (a) is providing an ankyrin repeat domain which has an isoelectric point (pI) higher than pH 4.07, and/or among all amino acid residues comprised in the repeat domain, a percentage of basic amino acid residues higher than 7.0%, and/or among the framework residues comprised in the repeat domain, a percentage of basic amino acid residues higher than 6.1%; and
   wherein step (b) is (1) substituting at least one basic amino acid residue of the repeat domain of step
   (a) with a neutral or acidic amino acid residue; and/or
   (2) substituting at least one neutral amino acid residue of the repeat domain of step (a) with an acidic amino acid;
      wherein said modified ankyrin repeat domain has at least one characteristic selected from the following list of characteristics:
(i) an isoelectric point (pI) equal to or lower than pH 4.07;
(ii) among all amino acid residues comprised in the repeat domain, a percentage of basic amino acid residues equal to or lower than 7.0%; and
(iii) among the framework residues comprised in the repeat domain, a percentage of basic amino acid residues equal to or lower than 6.1%.
E16. The method according to E15, wherein the method comprises steps (a) and (b),
   wherein step (a) is providing an ankyrin repeat domain which has an isoelectric point (pI) higher than 35 pH 4.07; and
   wherein step (b) is (1) substituting at least one basic amino acid residue of the repeat domain of step
   (a) with a neutral or acidic amino acid residue; and/or
   (2) substituting at least one neutral amino acid residue of the repeat domain of step (a) with an acidic amino acid;
      wherein said modified ankyrin repeat domain has an isoelectric point (pI) equal to or lower than pH 4.07.
E17. The method according to E15, wherein the method comprises steps (a) and (b),
   wherein step (a) is providing an ankyrin repeat domain which has, among all amino acid residues comprised in the repeat domain, a percentage of basic amino acid residues higher than 7.0%; and
   wherein step (b) comprises substituting at least one basic amino acid residue of the repeat domain of step (a) with a neutral or acidic amino acid residue;
      wherein said modified ankyrin repeat domain has, among all amino acid residues comprised in the repeat domain, a percentage of basic amino acid residues equal to or lower than 7.0%.
E18. The method according to E15, wherein the method comprises steps (a) and (b),
   wherein step (a) is providing an ankyrin repeat domain which has, among the framework residues comprised in the repeat domain, a percentage of basic amino acid residues higher than 6.1%; and
   wherein step (b) comprises substituting at least one basic amino acid residue among the framework residues of the repeat domain of step (a) with a neutral or acidic amino acid residue;
      wherein said modified ankyrin repeat domain has, among the framework residues comprised in the repeat domain, a percentage of basic amino acid residues equal to or lower than 6.1%.
E19. The method according to any one of E15 to E18, wherein the repeat domain in step (a) has a KR/DE ratio higher than 0.44 and the modified repeat domain has a KR/DE ratio equal to or lower than 0.44, and/or wherein the repeat domain in step (a) has a KR/DE ratio among the framework residues higher than 0.36 and the modified repeat domain has a KR/DE ratio equal to or lower than 0.36, and/or wherein the repeat domain in step (a) has a KR/DE ratio among all residues of the N-terminal capping module higher than 0.66 and the modified repeat domain has a KR/DE ratio among all residues of the N-terminal capping module equal to or lower than 0.66.
E20. The method according to any one of E15 to E19, wherein the repeat domain of step (a) binds specifically to a target, optionally with a dissociation constant (K_{D}) lower than 10⁻⁷ M.
E21. The method according to E20, wherein said modified repeat domain binds specifically said target with a K_{D} lower than 10⁻⁷ M, preferably wherein said modified repeat domain binds said target with a K_{D} that is (1) about equal to the K_{D} with which said repeat domain of step (a) binds said target or (2) less than 100 fold, less than 10 fold, less than 5 fold or less than 2 fold higher than the K_{D} with which said repeat domain of step (a) binds said target.
E22. The method according to any one of E15 to E21, wherein said modified repeat domain comprises one internal repeat module, two internal repeat modules, three internal repeat modules, or four internal repeat modules.
E23. A designed ankyrin repeat domain comprising an N-terminal capping module, at least one internal repeat module and a C-terminal capping module, wherein said repeat domain is linked to a drug moiety, and wherein said repeat domain has at least one characteristic selected from the following list of characteristics:
   (a) an isoelectric point (pI) equal to or lower than pH 4.6;
   (b) among all amino acid residues comprised in said repeat domain, a percentage of basic amino acid residues equal to or lower than 12.0%;
   (c) among all amino acid residues comprised in said repeat domain, a percentage of Arg and Lys residues equal to or lower than 8.0%;
   (d) among the framework residues comprised in said repeat domain, a percentage of basic amino acid residues equal to or lower than 9.6%; and
   (e) among the framework residues comprised in said repeat domain, a percentage of Arg and Lys residues equal to or lower than 6.7%.
E24. The repeat domain according to E23, wherein said isoelectric point (pI) is in a range between pH 3.0 and pH 5.0, preferably between pH 3.0 and pH 4.6, and more preferably between pH 3.50 and pH 4.53.
E25. The repeat domain according to any one of E23 to E24, wherein said repeat domain has at least characteristic (b), and wherein the percentage in (b) is optionally equal to or lower than 8.7%.
E26. The repeat domain according to any one of E23 to E25, wherein said repeat domain has at least characteristic (c), and wherein the percentage in (c) is optionally equal to or lower than 4.7%.
E27. The repeat domain according to any one of E23 to E26, wherein said repeat domain has at least characteristic (d), and wherein the percentage in (d) is optionally equal to or lower than 6.7%.
E28. The repeat domain according to any one of E23 to E27, wherein said repeat domain has at least characteristic (e), and wherein the percentage in (e) is optionally equal to or lower than 5.7%.
E29. The repeat domain according to any one of E23 to E28, wherein said repeat domain has a KR/DE ratio equal to or lower than 0.44, and/or wherein said repeat domain has a KR/DE ratio among the framework residues equal to or lower than 0.36, and/or wherein said repeat domain has a KR/DE ratio among all residues of the N-terminal capping module equal to or lower than 0.66.
E30. The repeat domain according to any one of E23 to E29, wherein said repeat domain has a total number of basic amino acid residues equal to or lower than n, wherein n=4+5R, wherein R is the number of internal repeat modules comprised in said repeat domain.
E31. The repeat domain according to any one of E23 to E30, wherein said repeat domain has a total number of Arg and Lys residues equal or lower than m, wherein m=5+2R, wherein R is the number of internal repeat modules comprised in said repeat domain.
E32. The repeat domain according to any one of E23 to E31, wherein said repeat domain has no Arg residue and no Lys residue within the framework residue positions.
E33. The repeat domain according to any one of E23 to E32, wherein said repeat domain comprises one internal repeat module, two internal repeat modules, three internal repeat modules, or four internal repeat modules.
E34. The repeat domain according to any one of E23 to E33, wherein said repeat domain binds specifically to a target.
E35. The repeat domain according to E34, wherein said repeat domain binds said target with a dissociation constant (K_{D}) lower than 10⁻⁷ M.
E36. The repeat domain according to any one of E23 to E35, wherein the drug moiety is a therapeutic moiety or a diagnostic moiety.
E37. The repeat domain according to any one of E23 to E36, wherein said drug moiety is a toxin.
E38. The repeat domain according to E37, wherein said toxin is a radionuclide.
E39. The repeat domain according to E37, wherein said toxin is a cytotoxin.
E40. The repeat domain according to any one of E23 to E39, wherein the repeat domain comprises an amino acid sequence selected from the group consisting of (1) SEQ ID NOs: 104 to 114 and (2) sequences with at least 80% amino acid sequence identity among the framework residues of any one of SEQ ID NOs: 104 to 114.
E41. The repeat domain according to any one of E23 to E40, wherein the repeat domain comprises an amino acid sequence selected from the group consisting of (1) SEQ ID NOs: 104 and 112 and (2) sequences with at least 80% amino acid sequence identity among the framework residues of any one of SEQ ID NOs: 104 and 112.
E42. The repeat domain according to any one of E23 to E41, wherein the repeat domain comprises an amino acid sequence selected from the group consisting of (1) SEQ ID NOs: 8 to 10, 12 and 95 to 98 and (2) sequences with at least 80% amino acid sequence identity with any one of SEQ ID NOs: 8 to 10, 12 and 95 to 98.
E43. A designed ankyrin repeat domain linked to a drug moiety according to any one of E23 to E42 for use in a method of treating and/or diagnosing a medical condition comprising the step of administering to a patient in need thereof a therapeutically and/or diagnostically effective amount of said repeat domain, and wherein optionally said administration is not an oral administration.
E44. The designed ankyrin repeat domain linked to a drug moiety for use in a method of treating and/or diagnosing a medical condition according to E43, wherein the medical condition is a cancer.
E45. A method of generating a modified ankyrin repeat domain linked to a drug moiety, the method comprising steps (a) and (b),
   wherein step (a) is providing an ankyrin repeat domain which has an isoelectric point (pI) higher than pH 4.6, and/or among all amino acid residues comprised in the repeat domain, a percentage of basic amino acid residues higher than 12.0%, and/or among all amino acid residues comprised in the repeat domain, a percentage of Arg and Lys residues higher than 8.0%, and/or among the framework residues comprised in the repeat domain, a percentage of basic amino acid residues higher than 9.6%, and/or among the framework residues comprised in the repeat domain, a percentage of Arg and Lys residues higher than 6.7%; and
   wherein step (b) is (1) substituting at least one basic amino acid residue of the repeat domain of step (a) with a neutral or acidic amino acid residue; and/or
   (2) substituting at least one neutral amino acid residue of the repeat domain of step (a) with an acidic amino acid;
      wherein said modified repeat domain has at least one characteristic selected from the following list of characteristics:
(i) an isoelectric point (pI) equal to or lower than pH 4.6;
(ii) among all amino acid residues comprised in the repeat domain, a percentage of basic amino acid residues equal to or lower than 12.0%;
(iii) among all amino acid residues comprised in the repeat domain, a percentage of Arg and Lys residues equal to or lower than 8.0%;
(iv) among the framework residues comprised in the repeat domain, a percentage of basic amino acid residues equal to or lower than 9.6%; and
(v) among the framework residues comprised in the repeat domain, a percentage of Arg and Lys residues equal to or lower than 6.7%.
E46. The method according to E45, wherein the method comprises steps (a) and (b),
   wherein step (a) is providing an ankyrin repeat domain which has an isoelectric point (pI) higher than pH 4.6; and
   wherein step (b) is (1) substituting at least one basic amino acid residue of the repeat domain of step
   (a) with a neutral or acidic amino acid residue; and/or
   (2) substituting at least one neutral amino acid residue of the repeat domain of step (a) with an acidic amino acid;
      wherein said modified repeat domain has an isoelectric point (pI) equal to or lower than pH 4.6;
E47. The method according to E45, wherein the method comprises steps (a) and (b),
   wherein step (a) is providing an ankyrin repeat domain which has, among all amino acid residues comprised in the repeat domain, a percentage of basic amino acid residues higher than 12.0%; and
   wherein step (b) comprises substituting at least one basic amino acid residue of the repeat domain of step (a) with a neutral or acidic amino acid residue;
      wherein said modified repeat domain has, among all amino acid residues comprised in the repeat domain, a percentage of basic amino acid residues equal to or lower than 12.0%.
E48. The method according to E45, wherein the method comprises steps (a) and (b),
   wherein step (a) is providing an ankyrin repeat domain which has, among all amino acid residues comprised in the repeat domain, a percentage of Arg and Lys residues higher than 8.0%; and
   wherein step (b) comprises substituting at least one Arg or Lys residue of the repeat domain of step (a) with a neutral or acidic amino acid residue;
      wherein said modified repeat domain has, among all amino acid residues comprised in the repeat domain, a percentage of Arg and Lys residues equal to or lower than 8.0%.
E49. The method according to E45, wherein the method comprises steps (a) and (b),
   wherein step (a) is providing an ankyrin repeat domain which has, among the framework residues comprised in the repeat domain, a percentage of basic amino acid residues higher than 9.6%; and
   wherein step (b) comprises substituting at least one basic amino acid residue among the framework residues of the repeat domain of step (a) with a neutral or acidic amino acid residue;
      wherein said modified repeat domain has, among the framework residues comprised in the repeat domain, a percentage of basic amino acid residues equal to or lower than 9.6%.
E50. The method according to E45, wherein the method comprises steps (a) and (b),
   wherein step (a) is providing an ankyrin repeat domain which has, among the framework residues comprised in the repeat domain, a percentage of Arg and Lys residues higher than 6.7%; and
   wherein step (b) comprises substituting at least one Arg or Lys residue among the framework residues of the repeat domain of step (a) with a neutral or acidic amino acid residue;
      wherein said modified repeat domain has, among the framework residues comprised in the repeat domain, a percentage of Arg and Lys residues equal to or lower than 6.7%.
E51. The method according to any one of E45 to E50, wherein the repeat domain in step (a) has a KR/DE ratio higher than 0.44 and the modified repeat domain has a KR/DE ratio equal to or lower than 0.44, and/or wherein the repeat domain in step (a) has a KR/DE ratio among the framework residues higher than 0.36 and the modified repeat domain has a KR/DE ratio equal to or lower than 0.36, and/or wherein the repeat domain in step (a) has a KR/DE ratio among all residues of the N-terminal capping module higher than 0.66 and the modified repeat domain has a KR/DE ratio among all residues of the N-terminal capping module equal to or lower than 0.66.
E52. The method according to any one of E45 to E51, wherein said method further comprises a step of linking a drug moiety to said modified repeat domain.
E53. The method according to any one of E45 to E52, wherein the repeat domain of step (a) binds specifically to a target, optionally with a dissociation constant (K_{D}) lower than 10⁻⁷ M.
E54. The method according to E53, wherein said modified repeat domain binds specifically said target with a K_{D} lower than 10⁻⁷ M, preferably wherein said modified repeat domain binds said target with a K_{D} that is (1) about equal to the K_{D} with which said repeat domain of step (a) binds said target or (2) less than 100 fold, less than 10 fold, less than 5 fold or less than 2 fold higher than the K_{D} with which said repeat domain of step (a) binds said target.
E55. The method according to any one of E45 to E54, wherein said modified repeat domain comprises one internal repeat module, two internal repeat modules, three internal repeat modules, or four internal repeat modules.
E56. A designed ankyrin repeat domain obtainable or obtained by the method according to any one of E15 to E22 or E45 to E55.
E57. A recombinant protein comprising the designed repeat domain according to any one of E1 to E14, E23 to E42 and E56.
E58. An isolated nucleic acid encoding the designed repeat domain according to any one of E1 to E14, E23 to E42 and E56 or the recombinant protein according to E57.
E59. A recombinant expression vector comprising the nucleic acid according to E58.
E60. A host cell comprising the recombinant expression vector according to E59.
E61. A pharmaceutical composition comprising one or more of: (i) the repeat domain according to any one of E1 to E140, E23 to E42 and E56, (ii) the recombinant protein according to E57, (iii) the nucleic acid according to E58, and/or (iv) the recombinant expression vector according to E59, and optionally at least one pharmaceutically acceptable carrier or diluent.
E62. A method of treating and/or diagnosing a medical condition, the method comprising the step of administering to a patient in need thereof a therapeutically and/or diagnostically effective amount of the repeat domain according to any one of E1 to E14, E23 to E42 and E56, the recombinant protein according to E57, the nucleic acid according to E58 or the pharmaceutical composition according to E61.
E63. The method of E62, wherein said medical condition is a cancer.
E64. The repeat domain according to any one of E1 to E14, E23 to E42 and E56, the recombinant protein according to E57, the nucleic acid according to E58 or the pharmaceutical composition according to E61, for use in a method of treating and/or diagnosing a medical condition.
E65. The repeat domain, the recombinant protein, the nucleic acid or the pharmaceutical composition for use according to E64, wherein said medical condition is a cancer.

### BRIEF DESCRIPTION OF THE FIGURES

FIGS. 1A-1B: Sequences and properties of representative DARPin variants according to the invention, the parental DARPins, a negative control DARPin variant (DARPin07), and the consensus DARPin are shown. Sequence names in bold underlined represent the parental DARPins which were taken as basis for the engineered variants. FIG. 1A shows some characteristics of the DARPins computed either on the entire repeat domain (i.e. by considering all residues comprised in the repeat domain) or on the framework positions only (i.e. by considering all residues comprised in the repeat domain except potential target interaction residues). The symbol “#” as used for instance in #(Arg+Lys) means the summed number of Arg and Lys residues. The symbol “%” as used for instance in % (Arg+Lys) means the percentage of Arg and Lys residues comprised in a repeat domain or the framework positions as indicated. As shown in FIG. 1B, the exemplary DARPin variants comprise an N-terminal capping module, a C-terminal capping module and two internal repeat modules. FIG. 1B is an alignment of all DARPin variant sequences, where randomized positions are marked by an underlined “X” in the consensus row (the full sequences are split into different boxes for practical reasons only). Some positions have a strong tendency for a certain acidic/basic residue based on the consensus sequence (such as position 10 in internal repeat module 1) but have been found to differ for the shown variants (e.g. due to mutations that increase affinity).

FIG. 2: Size exclusion chromatography (SEC) profiles for the tested parental DARPins and DARPin variants. Plots 1 to 12 show the SEC profiles of DARPin01 to DARPin12 (SEQ ID NOs: 1 to 12 respectively), where each DARPin additionally comprises a C-terminal GSGSC tag (SEQ ID NO: 14). All SEC profiles exhibit a dimeric peak before the main monomeric peak, due to the partial formation of disulfide-linked dimers (C-terminal Cys).

FIG. 3: Graphical summary of the production process of the radiolabeled DARPins according to the invention. DARPin variants were expressed in E. coli and purified over IMAC (immobilized metal affinity chromatography) and GF (gel filtration). Constructs were cleaved by recombinant TEV protease to cleave off the His-tag. Subsequently, non-cleaved DARPin variants as well as His-tagged TEV protease were removed by inverse IMAC, flow-through was collected and loaded on a SEC column. Purified DARPins were reduced and coupled with the chelator DTPA. Chelated DARPins were subsequently loaded with radionuclide indium-111 (also referred to as ¹¹¹In).

FIG. 4: Single-trace SPR profiles of DTPA-coupled DARPins (also referred to as constructs, see Table 7) against biotinylated full-length HER2. As reference, plots 1 and 2 show the profiles of the HER2-binding parental DARPin06 (SEQ ID NO: 6) and DARPin11 (SEQ ID NO: 11), respectively. Plots 3, 4, 5, 6 and 7 show the profiles of construct 6 (DARPin06-GSGSC-DTPA), construct 7 (DARPin07-GSGSC-DTPA), construct 8 (DARPin08-GSGSC-DTPA), construct 9 (DARPin09-GSGSC-DTPA) and construct 10 (DARPin10-GSGSC-DTPA), respectively. Plots 8 and 9 show construct 11 (DARPin11-GSGSC-DTPA) and construct 12 (DARPin12-GSGSC-DTPA), respectively. The GSGSC tag (SEQ ID NO: 14) was fused to the C-terminal end of the DARPins. All analytes (500 nM) were injected in succession for 120s, dissociation was recorded for 180s (25 ul/min of PBS-Tween20 (0.005%)). Each injection was followed by a regeneration step with glycine pH2.0 for 60s. The data was double referenced (control spot and buffer injection) and fitted to a 1:1 Langmuir model.

FIG. 5: Kidney uptake of indium-labeled DARPins. Radiolabeled DARPin01 to DARPin12 were injected in wild-type mice. Data are shown as mean % injected activity/gram of tissue mass (% IA/g). Measures were taken 4 hours after injection. Error bars show standard deviations. Bars represented in black correspond to the parental DARPins. DARPin07 is a negative control having a higher pI and higher percentage of basic amino acids among the framework residues as compared to its parental DARPin06.

FIGS. 6A-6C: Biodistribution study of ¹¹¹In-labeled DARPins in mice bearing HER2-expressing SKOV3ip tumors. Two DARPins with binding specificity for HER2 (i.e. DARPin06 and DARPin08) and two DARPins not binding to HER2 (i.e. DARPin01 and DARPin02) where tested in this study. Mice were injected with radiolabeled DARPins (1 mg/kg, approx. 150 KBq) two weeks (n=6 per group) or three weeks (n=6 per group) after SKOV3ip tumor cell implantation. Measurements were taken 4 hours after injection. Data are shown as mean % injected activity/gram of tissue mass (% IA/g, pooled n=12 per group). FIG. 6A: Kidney accumulation of pI-engineered DARPins radiolabeled with 111 In compared to ¹¹¹In-labelled parental DARPins. A strong reduction in kidney accumulation is observed for the engineered variants (DARPin02 and DARPin08) as compared to the respective parental DARPins (DARPin01 and DARPin06, respectively). More specifically, a reduction of 90% (DARPin02 vs DARPin01) and 78% (DARPin08 vs DARPin06) results from the pI-engineering of the DARPin variants. FIG. 6B: DARPin uptake in tumors. Significant accumulation of HER2-specific DARPins in the tumors was observed and the accumulation of DARPin08 in the tumor was comparable to its parental DARPin06. Only negligible unspecific accumulation of non-binding DARPin01 and DARPin02 was observed in the tumor. In 6A and 6B, results corresponding to engineered variants are represented in white bars, results corresponding to parental DARPins in black bars. FIG. 6C: Accumulation of the DARPins in further tissues or organs (blood, heart, lung, spleen, liver, small intestines, large intestines, muscle, bone and tail) was measured. Overall, accumulation of the engineered variants (DARPin02 and DARPin08) was similar to the respective parental DARPins (DARPin01 and DARPin06, respectively). Error bars show SD.

FIG. 7: Time course biodistribution study of ¹¹¹In-labeled DARPins (DARPin06 and DARPin08) in mice bearing HER2-expressing SKOV3ip tumors. Mice were injected with radiolabeled DARPins (1 mg/kg, approx. 150 KBq) at a tumor volume of approx. 350 mm³. Evolution of the radioactivity in isolated kidneys, tumors and blood samples was measured 1 h, 4 h, 24 h, 48 h and 96 h after injection. Data are shown as mean % injected activity/gram of tissue mass (% IA/g, n=4 per group). The area under the curve (AUC) in the kidney for charge-engineered DARPin08 is reduced by 76% compared to parental DARPin06. The AUCs in the tumor and blood remains comparable for the two DARPins.

FIGS. 8A-8B: Sequences and properties of further exemplary DARPin variants according to the invention, derived from parental DARPin06. FIG. 8A shows characteristics of the DARPins computed either on the entire repeat domain (i.e. by considering all residues comprised in the repeat domain) or on the framework positions only (i.e. by considering all residues comprised in the repeat domain except potential target interaction residues). As shown in FIG. 8B, these further exemplary DARPin variants comprise an N-terminal capping module, a C-terminal capping module and two internal repeat modules. Randomized positions are marked by an underlined “X” in the consensus row (the full sequences are split into different boxes for practical reasons only). Definitions and legend for FIGS. 8A-8B correspond to the ones of FIGS. 1A-1B.

FIGS. 9A-9F: Multi-trace SPR profiles of DTPA-coupled DARPins (also referred to as constructs, see Tables 7 and 16) against biotinylated full-length HER2. FIGS. 9A, 9B, 9C, 9D, 9E, and 9F show the profiles of construct 6 (DARPin06-GSGSC-DTPA), construct 8 (DARPin08-GSGSC-DTPA), construct 13 (DARPin13-GSGSC-DTPA), construct 14 (DARPin14-GSGSC-DTPA), construct 15 (DARPin15-GSGSC-DTPA) and construct 16 (DARPin16-GSGSC-DTPA), respectively. The GSGSC tag (SEQ ID NO: 14) was fused to the C-terminal end of the DARPins. Three-fold dilutions of the analytes (50, 16.667, 5.556, 1.852 and 0.617 nM) were injected for 300s, dissociation was recorded for 1500s (25 ul/min). Each injection was followed by a regeneration step with glycine pH 2.0 for 60s. Dissociation constants (K_{D}) were calculated from the globally fitted on- and off-rates using standard 1:1-Langmuir model (see Table 17).

FIGS. 10A-10B: Biodistribution study of ¹¹¹In-labeled DARPins in mice bearing HER2-expressing SKOV3ip tumors. All tested DARPins have binding specificity for HER2. DARPin06 is the parental DARPin and DARPins 08, 13, 14, 15 and 16 are charge-engineered variants according to the invention. Mice were injected with radiolabeled DARPins (1 mg/kg, approx. 150 KBq) at a tumor volume of approx. 350 mm³. Measurements were taken 4 hours after injection. Data are shown as mean % injected activity/gram of tissue mass (% IA/g, n=4 per group). FIG. 10A: Kidney accumulation of charge-engineered DARPins 08, 13, 14, 15 and 16 radiolabeled with 111 In compared to ¹¹¹In-labelled parental DARPin06. A strong reduction in kidney accumulation is observed for the engineered variants as compared to the parental DARPin. More specifically, a reduction of 82% (DARPin08 vs DARPin06), 90% (DARPin13 vs DARPin06), 85% (DARPin14 vs DARPin06), 93% (DARPin15 vs DARPin06) and 87% (DARPin16 vs DARPin06) results from the charge-engineering of the DARPin variants. FIG. 10B: DARPin uptake in tumors. Accumulation of HER2-specific DARPins in the tumors was observed and the accumulation of the charge-engineered DARPin variants in the tumor was comparable to that of the parental DARPin06. In 10A and 10B, results corresponding to engineered variants are represented in white bars, results corresponding to parental DARPins in black bars. Error bars show SD.

### DETAILED DESCRIPTION OF THE INVENTION

Designed ankyrin repeat domains are structural units of designed ankyrin repeat proteins. Designed repeat protein libraries, including designed ankyrin repeat protein libraries (WO2002020565; Binz et al., Nat. Biotechnol. 22, 575-582, 2004; Stumpp et al., Drug Discov. Today 13, 695-701, 2008), can be used for the selection of target-specific designed repeat domains that bind to their target with high affinity. Such target-specific designed repeat domains in turn can be used as valuable components of recombinant binding proteins for the treatment and/or diagnosis of diseases.

Designed ankyrin repeat proteins are a class of binding molecules which have the potential to overcome limitations of monoclonal antibodies, hence allowing novel therapeutic and/or diagnostic approaches. Such ankyrin repeat proteins may comprise a single designed ankyrin repeat domain, or may comprise a combination of two, three, four, five or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two, three, four, five or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

The inventors of the present invention have surprisingly discovered that pharmacokinetic properties of designed ankyrin repeat domains can be improved by reducing the number of positive charges comprised in such repeat domains and/or by reducing the isoelectric point (pI) of such repeat domains. More particularly, it was found that such designed repeat domains of the invention exhibit a reduced accumulation in the kidneys upon in vivo administration. Further, when a designed repeat domain of the invention is linked to a drug moiety, the drug moiety exhibits a reduced accumulation in the kidneys upon in vivo administration. For example, when a designed repeat domain of the invention is linked to a radioactive agent, there is reduced accumulation of radioactivity in the kidneys upon in vivo administration. This property is highly desirable since renal accumulation of a toxic drug moiety in the kidneys leads to nephrotoxicity. For this reason, renal accumulation of radioactivity constrains the use of radiolabeled DARPins in therapy or diagnostics, and similarly, renal accumulation of a cytotoxic agent constrains the use of cytotoxic agent-conjugated DARPins. The biophysical properties and the target-specific accumulation of designed ankyrin repeat proteins of the invention in tumors expressing the target are not significantly affected by the charge engineering. Designed ankyrin repeat domains of the invention therefore provide an improved tumor to kidney ratio. Accordingly, the improved repeat domains and recombinant proteins provided in the present invention open the door to new therapeutic and/or diagnostic applications, for instance in the field of nuclear medicine such as in radiopharmaceutical therapy or in the field of cancer therapy using cytotoxic agent-conjugated proteins.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise stated. No language in the specification should be construed as indicating any non-claimed element as essential to the practice of the disclosure. The term “about” as used herein is equivalent to ±10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The term “nucleic acid” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid may either be present in isolated form or be comprised in recombinant nucleic acid molecules or vectors.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coh), these bacteria can produce the polypeptide(s) encoded by this recombinant DNA. The correspondingly produced polypeptide or protein is called a recombinant polypeptide or recombinant protein.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

The term “target” refers to an individual molecule such as a nucleic acid, a polypeptide or protein, a carbohydrate, or any other naturally or non-naturally occurring molecule or moiety, including any part of such individual molecule, or complexes of two or more of such molecules. The target may be a whole cell or a tissue sample. Preferably, the target is a naturally occurring or non-natural polypeptide or a polypeptide containing chemical modifications, for example modified by natural or non-natural phosphorylation, acetylation, or methylation.

Patent application WO2002020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers.

The term “repeat domain” refers to a protein domain comprising two or more consecutive repeat modules as structural units, wherein said repeat modules have structural and sequence homology. Preferably, a repeat domain also comprises an N-terminal and/or a C-terminal capping module. For clarity, a capping module can be a repeat module. Such repeat domains, repeat modules, and capping modules, sequence motifs, as well as structural homology and sequence homology are well known to the practitioner in the art from examples of ankyrin repeat domains (Binz et al., J. Mol. Biol. 332, 489-503, 2003; Binz et al., 2004, loc. Cit.; WO2002020565; WO2012069655), leucine-rich repeat domains (WO2002020565), tetratricopeptide repeat domains (Main, E. R., Xiong, Y., Cocco, M. J., D′Andrea, L., Regan, L., Structure 11(5), 497-508, 2003), and armadillo repeat domains (WO2009040338). It is further well known to the practitioner in the art that such repeat domains are different from proteins comprising repeated amino acid sequences, where every repeated amino acid sequence is able to form an individual domain (for example FN3 domains of Fibronectin). The repeat domains can be binding domains.

The term “ankyrin repeat domain” refers to a repeat domain comprising two or more consecutive ankyrin repeat modules as structural units, wherein said ankyrin repeat modules have structural and sequence homology.

The term “designed” as used in designed repeat protein, designed repeat domain and the like refers to the property that such repeat proteins and repeat domains, respectively, are man-made and do not occur in nature. The binding domains of the instant invention are designed repeat domains. Preferably, a designed repeat domain of the invention is a designed ankyrin repeat domain.

The term “repeat modules” refers to the repeated amino acid sequence and structural units of the designed repeat domains, which are originally derived from the repeat units of naturally occurring repeat proteins.

Each repeat module comprised in a repeat domain is derived from one or more repeat units of a family or subfamily of naturally occurring repeat proteins, preferably the family of ankyrin repeat proteins. Furthermore, each repeat module comprised in a repeat domain may comprise a “repeat sequence motif” deduced from homologous repeat modules obtained from repeat domains selected on a target and having the same target specificity. A repeat module as used in the present invention encompasses internal repeat modules and capping modules such as N-terminal and C-terminal capping modules. An “internal repeat module” refers to a repeat module that is flanked by two repeat modules. In other words, an internal repeat module is N-terminally flanked by one repeat module and C-terminally flanked by another repeat module.

Accordingly, the term “ankyrin repeat module” refers to a repeat module, which is originally derived from the repeat units of naturally occurring ankyrin repeat proteins. Ankyrin repeat proteins are well known to the person skilled in the art. Designed ankyrin repeat proteins have been described previously; see, e.g., International Patent Publication WO2002020565, WO2010060748, WO2011135067, WO2012069654, WO2012069655, WO2014001442, WO2014191574, WO2014083208, WO2016156596, and WO2018054971, all of which are incorporated by reference in their entireties. Typically, an ankyrin repeat module comprises about 31 to 33 amino acid residues that form two alpha helices, separated by loops.

Repeat modules may comprise positions with amino acid residues which have not been randomized in a library for the purpose of selecting target-specific repeat domains (“non-randomized positions” or “fixed positions” used interchangeably herein) and positions with amino acid residues which have been randomized in the library for the purpose of selecting target-specific repeat domains (“randomized positions”). Non-randomized positions comprise framework residues and may also comprise target interaction residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, amino acid residues 3, 4, 6, 11, 14 and 15 of SEQ ID Nos: 26 to 35, 46 to 52, 61 to 62, 92 to 94 and 131 to 142, amino acid residues 4, 8, 11 and 12 of SEQ ID Nos: 15 to 25, 43 to 45, 57 to 60, 65 to 81, 99 to 101 and 115 to 122, and amino acid residues 3, 4, 6, 14 and 15 of SEQ ID Nos: 36 to 42, 53 to 56, 63 to 64, 82 to 91, 102 to 103 and 123 to 130 are randomized positions of the ankyrin repeat modules of the instant invention.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

A residue or amino acid residue refers to an amino acid comprised in a peptide chain. The term “target interaction residues” refers to amino acid residues of a repeat module, which contribute to the direct interaction with a target. Such contribution of a residue can be tested, e.g., in a binding assay, for example in a mutagenesis study performed to identify residues required, sufficient, and/or necessary for a repeat domain to bind a target with its original binding affinity or quantity (i.e. its binding affinity or quantity in the absence of any mutations). Target interaction residues can also be determined by structural analyses of a repeat domain bound to a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops. Specifically for the purpose of calculating (1) the percentage of basic amino acid residues among the framework residues comprised in a designed ankyrin repeat domain of the invention, or (2) the percentage of Arg and Lys residues together among the framework residues comprised in a designed ankyrin repeat domain of the invention, the term “framework residues” includes the amino acid residues located at the positions within the designed ankyrin repeat domain that correspond to the positions listed in Table 4 for the representative N-terminal capping module (i.e. positions 1 to 3, 5 to 7, 9, 10, and 13 to 30 of SEQ ID NO: 43), the representative internal repeat module (i.e. positions 1, 2, 5, 7 to 10, 12, 13, and 16 to 33 of SEQ ID NO: 48) and the representative C-terminal capping module (i.e. positions 1, 2, 5, 7 to 13, and 16 to 28 of SEQ ID NO: 54). The term “framework residues” does not include the amino acid residues located at the positions within the designed ankyrin repeat domain that correspond to the positions listed in Table 5 for the representative N-terminal capping module (i.e. positions 4, 8, 11 and 12 of SEQ ID NO: 43), the representative internal repeat module (i.e. positions 3, 4, 6, 11, 14 and 15 of SEQ ID NO: 48) and the representative C-terminal capping module (i.e. positions 3, 4, 6, 14 and 15 of SEQ ID NO: 54).

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “binding specificity”, “has binding specificity for a target”, “specifically binding to a target”, “binding to a target with high specificity”, “specific for a target” or “target specificity” and the like means that a binding protein or binding domain binds to a target with a lower dissociation constant (i.e. it binds with higher affinity) than it binds to an unrelated protein such as the E. coli maltose binding protein (MBP). Preferably, the dissociation constant (“K_{D}”) for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS.

Binding of any molecule to another is governed by two forces, namely the association rate (Icon) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {{⇌}^{{k}_{on}}}_{{k}_{off}} [ BT ]$$

kₒₙ is a second-order rate constant of the binding reaction, with the unit M⁻¹ s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015 May 5; 15(5):10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). A K_{D} value may be measured using OCTET® technology (Octet Qke system, ForteBio). Alternatively, or in addition, a KinExA® (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred. Most preferably, the K_{D} values are determined in PBS and by SPR.

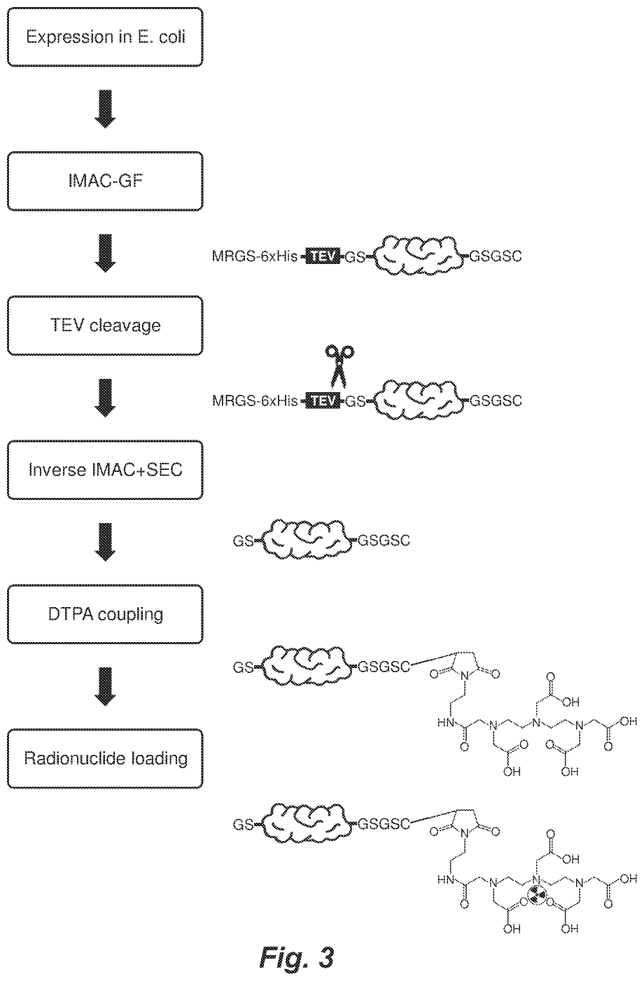

The term “Isoelectric point” or “pI” refers to the pH value at which a macromolecule such as a protein carries no net electrical charge. In proteins there may be many charged groups, and at the isoelectric point the sum of all these charges is zero. At a pH above the isoelectric point the overall net charge of the polypeptide will be negative, whereas at pH values below the isoelectric point the overall net charge of the polypeptide will be positive. Isoelectric points can be determined experimentally or can be calculated for polypeptides based on the primary sequence. The skilled person is aware of methods to determine the isoelectric point of a protein. Most commonly, the isoelectric point of a protein is computed based on the amino acid sequence of the protein. Numerous tools are available online that allow computing the isoelectric point of a protein. One such preferred tool is “ExPASy Compute pI/Mw” (https://web.expasy.org/computepi/); see Protein Identification and Analysis Tools on the ExPASy Server; Gasteiger E., Hoogland C., Gattiker A., Duvaud S., Wilkins M. R., Appel R. D., Bairoch A.; (In) John M. Walker (ed): The Proteomics Protocols Handbook, Humana Press (2005), pp. 571-607. This “ExPASy Compute pI/Mw” tool is preferably used for the determination of the pI of ankyrin repeat domains of the invention. Any N-terminal or C-terminal tags comprising one or more amino acids which may be fused to a repeat domain for production or other purposes, as well as any N-terminal or C-terminal peptide linkers are not considered for computing the pI of the repeat domains of the invention. Such tags or linkers are well known in the art and include for instance the His6-TEV tag of SEQ ID NO: 13 (N-terminal), the GS residues (N-terminal), and the GSGSC tag of SEQ ID NO: 14 (C-terminal), as shown in FIG. 3.

The term “basic amino acid” refers to a hydrophilic amino acid having a positively charged side chains at physiological pH. From the 20 common amino acids, His (H), Arg (R), and Lys (K) are basic amino acids (see Table 1). The term “acidic amino acid” refers to a hydrophilic amino acid having a negatively charged side chains at physiological pH. From the 20 common amino acids, Asp (D) and Glu (E) are acidic amino acids (see Table 1). Basic and acidic amino acids can also be collectively referred to as charged amino acids, since at physiological pH their side chains are ionized. “Neutral amino acid” refers to amino acids which are neither basic nor acidic, and are hence effectively non-ionized under physiological conditions. 15 of the 20 common amino acids are neutral amino acids (see Table 1). Such considerations are well known to a skilled in the art.

The term “drug moiety” refers to a chemical moiety that is linked or is suitable for linkage to a protein, in particular to a designed repeat domain or designed repeat protein, and includes any therapeutic or diagnostic agent that has desired therapeutic or diagnostic properties, respectively. Drug moiety as used herein encompasses the terms “therapeutic moiety” and “diagnostic moiety”. Such moieties can be linked to repeat domains or repeat proteins of the invention using methods available in the art, or for instance as described in Example 3.

The term “therapeutic moiety” refers to a chemical moiety that can function as a therapeutic agent (or perform a therapeutic function), such as for a treatment of a disease or disorder when administered to or otherwise provided to a patient or subject.

The term “diagnostic moiety” refers to a chemical moiety that can function as a diagnostic agent (or perform a diagnostic function), such as for a diagnosis of a disease or disorder when administered to or otherwise provided to a patient or subject.

The term “linked” or “linkage” refers to any covalent or non-covalent linkage between a chemical moiety and a protein such as a designed repeat domain or a designed repeat protein.

The term “toxin” refers to any agent that is detrimental to the growth, proliferation and/or survival of cells and may act to reduce, inhibit, kill and/or destroy a cell or malignancy. This term encompasses for instance a radionuclide, which may be toxic because of its radioactivity, and a cytotoxic agent.

The term “cytotoxic agent” or “cytotoxin” refers to a substance that causes cell death or toxicity primarily by interfering with a cell's vital processes, such as for example gene expression activity, DNA replication, cell division, and/or cell survival. Non-limiting examples of cytotoxins include chemotherapeutic agents, mitotic inhibitors, growth inhibitory agents, enzymes and fragments thereof such as nucleolytic enzymes, antibiotics, toxins or enzymatically active toxins of bacterial, fungal, plant or animal origin, auristatins, calicheamicins, maytansinoids and camptothecin analogues. Further non-limiting examples of cytotoxins are cytotoxins which can be used in antibody-drug conjugates as described e.g. in Drago, Joshua Z., Shanu Modi, and Sarat Chandarlapaty. Nature Reviews Clinical Oncology 18.6 (2021).

The term “radionuclide” or “radioisotope” refers to isotopes of natural or artificial origin with an unstable neutron to proton ratio that disintegrates with the emission of corpuscular (i.e. protons (alpha-radiation) or electrons (beta-radiation)) or electromagnetic radiation (gamma-radiation). In other words, radionuclides undergo radioactive decay. Such radionuclides include, without limitation, ⁹⁴ Tc, ⁹⁹ mTc, ⁹⁰In, ¹¹¹In, ⁶⁷Ga, ⁶⁸Ga, ⁸⁶Y, ⁹⁰Y, ¹⁷⁷Lu, ¹⁵¹ Tb, ²²³Ra, ¹⁸⁶Re, ¹⁸⁸Re, ⁶⁴Cu, ⁶⁷ Cu, ⁵⁵Co, ⁵⁷Co, ⁴³Sc, ⁴⁴Sc, ⁴⁷Sc, ²³⁵Ac, ²¹³Bi, ²¹²Bi, ²¹²Pb, ²²⁷ Th, ¹⁵³ Sm, ¹⁶⁶Ho, ¹⁵²Gd, ¹⁵³ Gd, ¹⁵⁷ Gd, ²²⁵Ac or ¹⁶⁶Dy. The choice of suitable radionuclides may depend on the chemical structure and chelating capability of the chelating agent, and the intended application of the resulting drug (e.g. diagnostic vs. therapeutic).

The terms “chelator” or “chelating agent” refer to polydentate (multiple bonded) ligands capable of forming two or more separate coordinate bonds with (“coordinating”) a central (metal) ion. Specifically, such molecules or molecules sharing one electron pair may also be referred to as “Lewis bases”. The central (metal) ion is usually coordinated by two or more electron pairs to the chelating agent. Usually, the electron pairs of a chelating agent forms coordinate bonds with a single central (metal) ion; however, in certain examples, a chelating agent may form coordinate bonds with more than one metal ion, with a variety of binding modes being possible. The terms “coordinating” and “coordination” refer to an interaction in which one multi-electron pair donor coordinatively bonds (“is coordinated”) to, i.e. shares two or more unshared pairs of electrons with, one central (metal) ion. The chelating agent is preferably chosen based on its ability to coordinate the desired central (metal) ion, usually a radionuclide as specified herein.

The term “percentage” as used for instance in percentage of basic amino acid residues means the value obtained by dividing the number of basic residues comprised in a considered sequence (such as, e.g., (1) the entire sequence of a repeat domain or (2) all framework residues comprised in a repeat domain) by the total number of amino acid residues comprised in said considered sequence, and multiplying the result by 100 to yield the percentage. Any N-terminal or C-terminal tags comprising one or more amino acids which may be fused to a repeat domain for production or other purposes, as well as any N-terminal or C-terminal peptide linker are not considered for computing such percentage. Such tags or linkers are well known in the art and include for instance the His6-TEV tag of SEQ ID NO: 13 (N-terminal), the GS residues (N-terminal), and the GSGSC tag of SEQ ID NO: 14 (C-terminal), as shown in FIG. 3.

The term “physiological conditions” refers to conditions normally present in a mammalian body. Thus, for example for humans, physiological conditions mean a pH between 7.35 and 7.45, with the average at 7.40, and a temperature between 36.1° C. and 37.2° C., with the average at 37° C.

Repeat Domains of the Invention

DARPins (designed ankyrin repeat proteins) are repeat proteins comprising a designed ankyrin repeat domain, which typically comprises one or more internal repeat modules that are identical or similar except for their randomized positions and that are flanked by an N-terminal and a C-terminal capping repeat module (see, e.g., Binz et al. 2003, loc. cit.). As used herein, the term “repeat module” encompasses internal repeat modules and terminal repeat modules (C-cap and N-cap modules). 27 of the 33 amino acid positions of typical internal repeat modules are highly conserved, whereas the other 6 amino acid positions are less conserved and to the most part responsible for the specific interaction of the ankyrin repeat domain with its target (Binz et al. 2003, loc. cit.). The paratope of the ankyrin repeat domain is formed by the continuous surface formed largely by these variable positions of the internal repeat modules and sometimes also the capping repeat modules. DARPins also encompass proteins which comprise multiple designed ankyrin repeat domains linked together by appropriate linkers. Such linkers are known to the person skilled in the art.

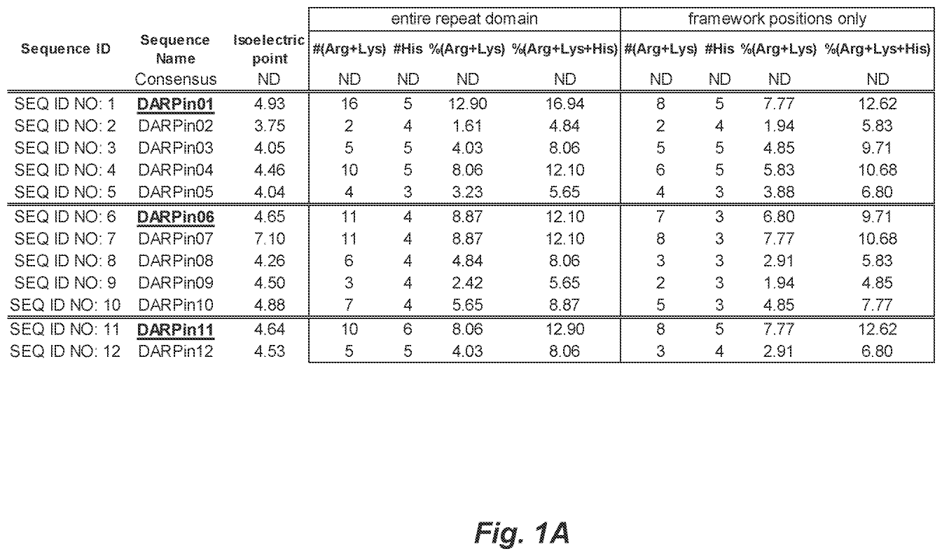

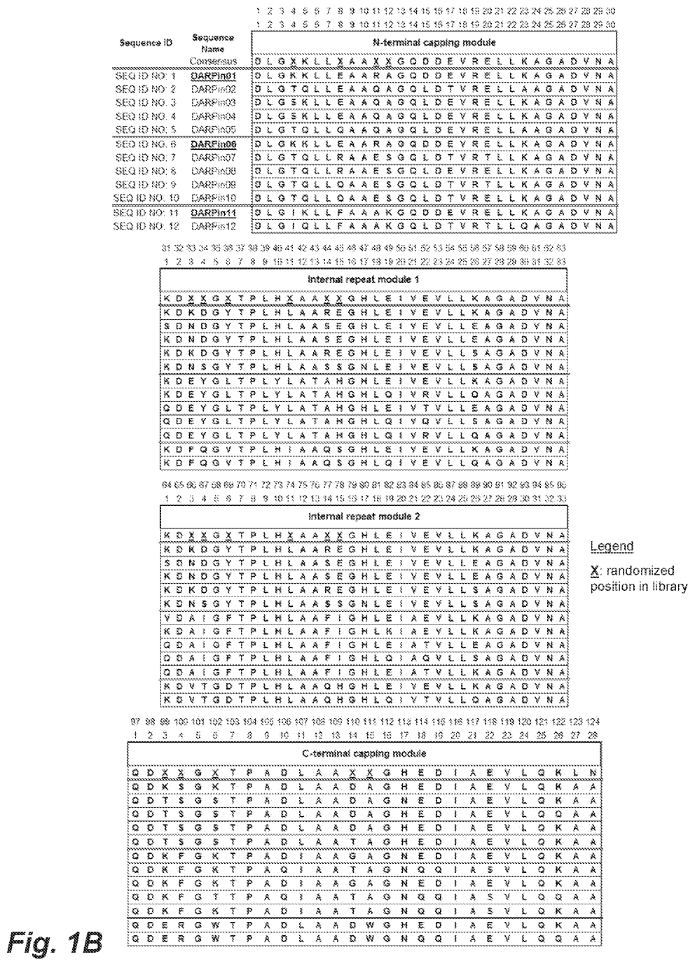

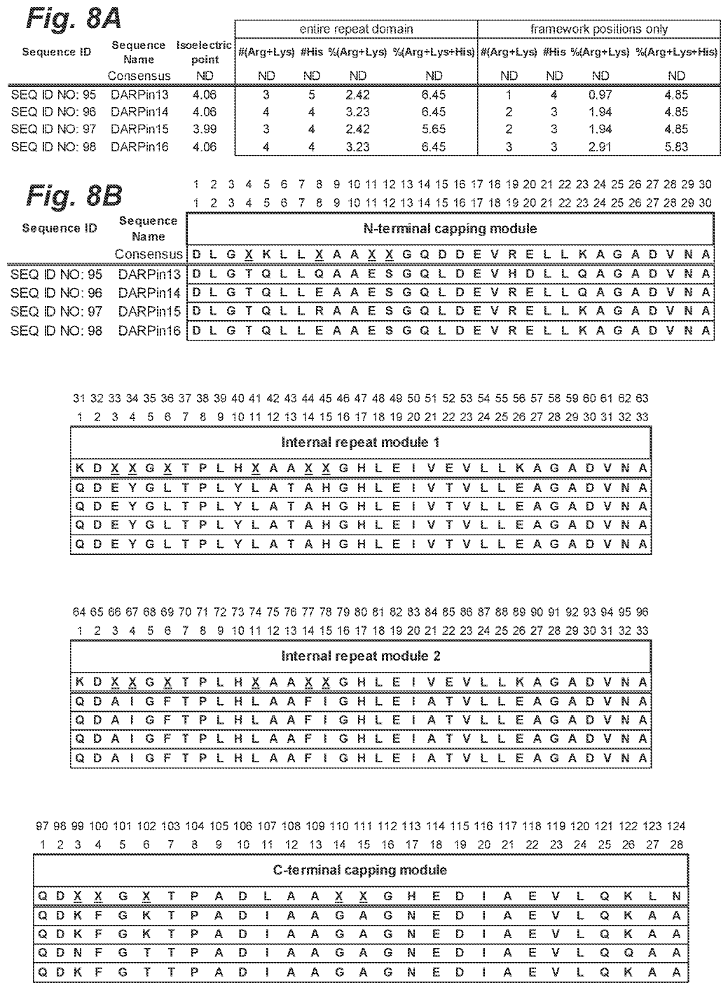

Representative designed ankyrin repeat domains according to the invention have been generated and characterized as described in Examples 1 to 8, and are shown in FIGS. 1A-1B and FIGS. 8A-8B.

Accordingly, in one aspect, the invention provides a designed ankyrin repeat domain having a reduced number of basic amino acid residues and/or a reduced pI as compared to a conventional designed ankyrin repeat domain. As will be described in more details below, designed ankyrin repeat domains of the invention may be obtained by substitution of basic amino acids, methods to perform such substitutions are well known in the art and include mutagenesis of the cDNA encoding the described repeat domains.

The ankyrin repeat domains described herein generally comprise a core scaffold that provides structure, and target interaction residues that bind to a target. The structural core includes conserved amino acid residues, also referred to as framework residues, and the target binding surface includes target interaction residues that differ depending on the target.

Table 1 shows typical properties of common amino acids as considered in the present specification.

TABLE 1

| | | Charge (under physiological |
|---|---|---|
| Amino acid | Category | conditions) |
| Arginine, Arg, R | basic | positive |
| Lysine, Lys, K | basic | positive |
| Histidine, His, H | basic | positive |
| Glutamate, Glu, E | acidic | negative |
| Aspartate, Asp, D | acidic | negative |
| Glycine, Gly, G | neutral | none |
| Alanine, Ala, A | neutral | none |
| Proline, Pro, P | neutral | none |
| Valine, Val, V | neutral | none |
| Leucine, Leu, L | neutral | none |
| Isoleucine, Ile, I | neutral | none |
| Methionine, Met, M | neutral | none |
| Phenylalanine, Phe, F | neutral | none |
| Tyrosine, Tyr, Y | neutral | none |
| Tryptophan, Trp, W | neutral | none |
| Serine, Ser, S | neutral | none |
| Threonine, Thr, T | neutral | none |
| Cysteine, Cys, C | neutral | none |
| Asparagine, Asn, N | neutral | none |
| Glutamine, Gln, Q | neutral | none |

Categories of charge-modifying amino acid residue substitutions considered in this specification and their impact on charge are shown in Table 2.

TABLE 2

| Original | Substitution | Impact on charge |
|---|---|---|
| basic | neutral | decrease |
| neutral | acidic | decrease |
| basic | acidic | highest decrease |

Examples of conservative and other exemplary amino acid residue substitutions that may occur in repeat domains and proteins of the invention are shown in Table 3.

TABLE 3

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

In some embodiments, the designed ankyrin repeat domain of the invention has a pI equal to or lower than pH 4.9, equal to or lower than pH 4.8, equal to or lower than pH 4.7, equal to or lower than pH 4.6, equal to or lower than pH 4.5, equal to or lower than pH 4.4, equal to or lower than pH 4.3, equal to or lower than pH 4.2, equal to or lower than pH 4.1, equal to or lower than pH 4.0, equal to or lower than pH 3.9, equal to or lower than pH 3.8, equal to or lower than pH 3.7, equal to or lower than pH 3.6, or equal to or lower than pH 3.5.

In some embodiments, the designed ankyrin repeat domain of the invention has a pI equal to or lower than pH 4.53, equal to or lower than pH 4.52, equal to or lower than pH 4.50, equal to or lower than pH 4.48, equal to or lower than pH 4.46, equal to or lower than pH 4.44, equal to or lower than pH 4.42, equal to or lower than pH 4.40, equal to or lower than pH 4.38, equal to or lower than pH 4.36, equal to or lower than pH 4.34, equal to or lower than pH 4.32, equal to or lower than pH 4.30, equal to or lower than pH 4.28, equal to or lower than pH 4.26, equal to or lower than pH 4.24, equal to or lower than pH 4.22, equal to or lower than pH 4.20, equal to or lower than pH 4.18, equal to or lower than pH 4.16, equal to or lower than pH 4.14, equal to or lower than pH 4.12, equal to or lower than pH 4.10, equal to or lower than pH 4.08, equal to or lower than pH 4.06, equal to or lower than pH 4.04, equal to or lower than pH 4.02, equal to or lower than pH 4.00, equal to or lower than pH 3.98, equal to or lower than pH 3.96, equal to or lower than pH 3.94, equal to or lower than pH 3.92, equal to or lower than pH 3.90, equal to or lower than pH 3.88, equal to or lower than pH 3.86, equal to or lower than pH 3.84, equal to or lower than pH 3.82, equal to or lower than pH 3.80, equal to or lower than pH 3.78, equal to or lower than pH 3.76, equal to or lower than pH 3.74, equal to or lower than pH 3.72, equal to or lower than pH 3.70, equal to or lower than pH 3.68, equal to or lower than pH 3.66, equal to or lower than pH 3.64, equal to or lower than pH 3.62, equal to or lower than pH 3.60, equal to or lower than pH 3.58, equal to or lower than pH 3.56, equal to or lower than pH 3.54, equal to or lower than pH 3.52, or equal to or lower than pH 3.50.

In some embodiments, the designed ankyrin repeat domain of the invention has a pI in a range between pH 3.00 and pH 4.60, between pH 3.10 and pH 4.60, between pH 3.20 and pH 4.60, between pH 3.30 and pH 4.60, between pH 3.40 and pH 4.60, between pH 3.50 and pH 4.60, between pH 3.50 and pH 4.58, between pH 3.50 and pH 4.56, between pH 3.50 and pH 4.54, between pH 3.50 and pH 4.53, between pH 3.50 and pH 4.52, between pH 3.50 and pH 4.50, between pH 3.50 and pH 4.48, between pH 3.50 and pH 4.46, between pH 3.50 and pH 4.44, between pH 3.50 and pH 4.42, between pH 3.50 and pH 4.40, between pH 3.50 and pH 4.38, between pH 3.50 and pH 4.36, between pH 3.50 and pH 4.34, between pH 3.50 and pH 4.32, between pH 3.50 and pH 4.30, between pH 3.60 and pH 4.60, between pH 3.60 and pH 4.58, between pH 3.60 and pH 4.56, between pH 3.60 and pH 4.54, between pH 3.60 and pH 4.53, between pH 3.60 and pH 4.52, between pH 3.60 and pH 4.50, between pH 3.60 and pH 4.48, between pH 3.60 and pH 4.46, between pH 3.60 and pH 4.44, between pH 3.60 and pH 4.42, between pH 3.60 and pH 4.40, between pH 3.60 and pH 4.38, between pH 3.60 and pH 4.36, between pH 3.60 and pH 4.34, between pH 3.60 and pH 4.32, between pH 3.60 and pH 4.30, between pH 3.70 and pH 4.60, between pH 3.70 and pH 4.58, between pH 3.70 and pH 4.56, between pH 3.70 and pH 4.54, between pH 3.70 and pH 4.53, between pH 3.70 and pH 4.52, between pH 3.70 and pH 4.50, between pH 3.70 and pH 4.48, between pH 3.70 and pH 4.46, between pH 3.70 and pH 4.44, between pH 3.70 and pH 4.42, between pH 3.70 and pH 4.40, between pH 3.70 and pH 4.38, between pH 3.70 and pH 4.36, between pH 3.70 and pH 4.34, between pH 3.70 and pH 4.32, or between pH 3.70 and pH 4.30.

In some embodiments, the designed ankyrin repeat domain of the invention has a percentage of basic amino acid residues equal to or lower than 12.0%, equal to or lower than 11.5%, equal to or lower than 11.0%, equal to or lower than 10.5%, equal to or lower than 10.0%, equal to or lower than 9.5%, or equal to or lower than 9.0%.

In some embodiments, the designed ankyrin repeat domain of the invention has a percentage of basic amino acid residues equal to or lower than 8.87%, equal to or lower than 8.80%, equal to or lower than 8.70%, equal to or lower than 8.60%, equal to or lower than 8.50%, equal to or lower than 8.40%, equal to or lower than 8.30%, equal to or lower than 8.20%, equal to or lower than 8.10%, equal to or lower than 8.00%, equal to or lower than 7.90%, equal to or lower than 7.80%, equal to or lower than 7.70%, equal to or lower than 7.60%, equal to or lower than 7.50%, equal to or lower than 7.40%, equal to or lower than 7.30%, equal to or lower than 7.20%, equal to or lower than 7.10%, equal to or lower than 7.00%, equal to or lower than 6.90%, equal to or lower than 6.80%, equal to or lower than 6.70%, equal to or lower than 6.60%, equal to or lower than 6.50%, equal to or lower than 6.40%, equal to or lower than 6.30%, equal to or lower than 6.20%, equal to or lower than 6.10%, equal to or lower than 6.00%, equal to or lower than 5.90%, equal to or lower than 5.80%, equal to or lower than 5.70%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, or equal to or lower than 4.80%.

Histidine residues under physiological conditions are typically partially positively charged. The impact on charge reduction resulting from a mutation of His to a neutral or acidic residue is less pronounced than mutating a Lys or Arg residue to a neutral or acidic residue. Unless specifically indicated otherwise, basic amino acid residues include Arg, Lys and His.

In some embodiments, the designed ankyrin repeat domain of the invention has a percentage of Arg and Lys residues equal to or lower than 8.0%, equal to or lower than 7.5%, equal to or lower than 7.0%, equal to or lower than 6.5%, or equal to or lower than 6.0%.

For clarity, the terms “percentage of Arg and Lys residues” as used in the present disclosure shall mean the percentage of Arg residues and Lys residues together.

In some embodiments, the designed ankyrin repeat domain of the invention has a percentage of Arg and Lys residues equal to or lower than 5.65%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, equal to or lower than 4.80%, equal to or lower than 4.70%, equal to or lower than 4.60%, equal to or lower than 4.50%, equal to or lower than 4.40%, equal to or lower than 4.30%, equal to or lower than 4.20%, equal to or lower than 4.10%, equal to or lower than 4.00%, equal to or lower than 3.90%, equal to or lower than 3.80%, equal to or lower than 3.70%, equal to or lower than 3.60%, equal to or lower than 3.50%, equal to or lower than 3.40%, equal to or lower than 3.30%, equal to or lower than 3.20%, equal to or lower than 3.10%, equal to or lower than 3.00%, equal to or lower than 2.90%, equal to or lower than 2.80%, equal to or lower than 2.70%, equal to or lower than 2.60%, equal to or lower than 2.50%, equal to or lower than 2.40%, equal to or lower than 2.30%, equal to or lower than 2.20%, equal to or lower than 2.10%, equal to or lower than 2.00%, equal to or lower than 1.90%, equal to or lower than 1.80%, equal to or lower than 1.70%, equal to or lower than 1.60%, equal to or lower than 1.50%, equal to or lower than 1.40%, equal to or lower than 1.30%, equal to or lower than 1.20%, equal to or lower than 1.10%, equal to or lower than 1.00%, equal to or lower than 0.90%, equal to or lower than 0.80%, equal to or lower than 0.70%, equal to or lower than 0.60%, equal to or lower than 0.50%, equal to or lower than 0.40%, equal to or lower than 0.30%, equal to or lower than 0.20%, or equal to or lower than 0.10%.

The number of basic amino acid residues in the designed ankyrin repeat domain of the invention may be alternatively defined with regards to the position of said basic amino acid residues within the repeat domain. In some embodiments, the basic amino acid number is considered among the framework residues of the repeat domain only. The positions of framework residues can be determined by the skilled person in the art. Preferably, framework residues shall correspond to residues occupying specific positions within the repeat modules as described in Table 4:

TABLE 4

| | Representative and | Position of framework |
|---|---|---|
| Repeat modules | reference sequence | residues |
| N-terminal capping | SEQ ID NO: 43 | 1 to 3, 5 to 7, |
| module | | 9, 10, 13 to 30 |
| Internal repeat | SEQ ID NO: 48 | 1, 2, 5, 7 to 10, |
| module | | 12, 13, 16 to 33 |
| C-terminal capping | SEQ ID NO: 54 | 1, 2, 5, 7 to 13, 16 to 28 |
| module | | |

In some embodiments, the designed ankyrin repeat domain of the invention has a percentage of basic amino acid residues among the framework residues equal to or lower than 9.6%, equal to or lower than 9.4%, equal to or lower than 9.2%, equal to or lower than 9.0%, equal to or lower than 8.8%, equal to or lower than 8.6%, equal to or lower than 8.4%, equal to or lower than 8.2%, or equal to or lower than 8.0%, equal to or lower than 7.8%.

In some embodiments, the designed ankyrin repeat domain of the invention has a percentage of basic amino acid residues among the framework residues equal to or lower than 7.77%, equal to or lower than 7.70%, equal to or lower than 7.60%, equal to or lower than 7.50%, equal to or lower than 7.40%, equal to or lower than 7.30%, equal to or lower than 7.20%, equal to or lower than 7.10%, equal to or lower than 7.00%, equal to or lower than 6.90%, equal to or lower than 6.80%, equal to or lower than 6.70%, equal to or lower than 6.60%, equal to or lower than 6.50%, equal to or lower than 6.40%, equal to or lower than 6.30%, equal to or lower than 6.20%, equal to or lower than 6.10%, equal to or lower than 6.00%, equal to or lower than 5.90%, equal to or lower than 5.80%, equal to or lower than 5.70%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, equal to or lower than 4.80%, equal to or lower than 4.70%, equal to or lower than 4.60%, or equal to or lower than 4.50%.

In some embodiments, the designed ankyrin repeat domain of the invention has a percentage of Arg and Lys residues among the framework residues equal to or lower than 6.7%, equal to or lower than 6.6%, equal to or lower than 6.5%, equal to or lower than 6.4%, equal to or lower than 6.3%, equal to or lower than 6.2%, equal to or lower than 6.1%, equal to or lower than 6.0%, or equal to or lower than 5.9%.

In some embodiments, the designed ankyrin repeat domain of the invention has a percentage of Arg and Lys residues among the framework residues equal to or lower than 5.83%, equal to or lower than 5.80%, equal to or lower than 5.70%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, equal to or lower than 4.80%, equal to or lower than 4.70%, equal to or lower than 4.60%, equal to or lower than 4.50%, equal to or lower than 4.40%, equal to or lower than 4.30%, equal to or lower than 4.20%, equal to or lower than 4.10%, equal to or lower than 4.00%, equal to or lower than 3.90%, equal to or lower than 3.80%, equal to or lower than 3.70%, equal to or lower than 3.60%, equal to or lower than 3.50%, equal to or lower than 3.40%, equal to or lower than 3.30%, equal to or lower than 3.20%, equal to or lower than 3.10%, equal to or lower than 3.00%, equal to or lower than 2.90%, equal to or lower than 2.80%, equal to or lower than 2.70%, equal to or lower than 2.60%, equal to or lower than 2.50%, equal to or lower than 2.40%, equal to or lower than 2.30%, equal to or lower than 2.20%, equal to or lower than 2.10%, equal to or lower than 2.00%, equal to or lower than 1.90%, equal to or lower than 1.80%, equal to or lower than 1.70%, equal to or lower than 1.60%, equal to or lower than 1.50%, equal to or lower than 1.40%, equal to or lower than 1.30%, equal to or lower than 1.20%, equal to or lower than 1.10%, equal to or lower than 1.00%, equal to or lower than 0.90%, equal to or lower than 0.80%, equal to or lower than 0.70%, equal to or lower than 0.60%, equal to or lower than 0.50%, equal to or lower than 0.40%, equal to or lower than 0.30%, equal to or lower than 0.20%, or equal to or lower than 0.10%.

Designed ankyrin repeat domains of the invention may comprise an N-terminal capping module, a C-terminal capping module and at least one internal repeat module. Preferably, said repeat domains of the invention comprise 1, 2, 3, 4, 5, 6, 7, 8 or 9 internal repeat module(s). More preferably, said repeat domains of the invention comprise 1, 2, 3 or 4 internal repeat modules.

In some embodiments, the total number of basic amino acid residues comprised in the designed ankyrin repeat domain of the invention can be expressed as a function based on the number of repeat modules comprised in said repeat domain. In one embodiment, the ankyrin repeat domain of the invention has a total number of basic amino acid residues equal to or lower than n, wherein n=R, n=1+R, n=2+R, n=3+R, n=4+R, n=5+R, n=6+R, n=7+R, n=8+R, n=9+R, n=2R, n=1+2R, n=2+2R, n=3+2R, n=4+2R, n=5+2R, n=6+2R, n=7+2R, n=8+2R, n=9+2R, n=3R, n=1+3R, n=2+3R, n=3+3R, n=4+3R, n=5+3R, n=6+3R, n=7+3R, n=8+3R, n=9+3R, n=4R, n=1+4R, n=2+4R, n=3+4R, n=4+4R, n=5+4R, n=6+4R, n=7+4R, n=8+4R, n=9+4R, n=5R, n=1+5R, n=2+5R, n=3+5R, n=4+5R, n=5+5R, n=6+5R, n=7+5R, n=8+5R, n=9+5R, n=6R, n=1+6R, n=2+6R, n=3+6R, n=4+6R, n=5+6R, n=6+6R, n=7+6R, n=8+6R, or n=9+6R, wherein R is the number of internal repeat modules comprised in said repeat domain. In such embodiments, “xR” means x multiplied by R.

In another embodiment, the total number of Arg and Lys residues comprised in the designed ankyrin repeat domain of the invention is equal to or lower than m, wherein m=R, m=1+R, m=2+R, m=3+R, m=4+R, m=5+R, m=6+R, m=7+R, m=8+R, m=9+R, m=2R, m=1+2R, m=2+2R, m=3+2R, m=4+2R, m=5+2R, m=6+2R, m=7+2R, m=8+2R, m=9+2R, m=3R, m=1+3R, m=2+3R, m=3+3R, m=4+3R, m=5+3R, m=6+3R, m=7+3R, m=8+3R, m=9+3R, m=4R, m=1+4R, m=2+4R, m=3+4R, m=4+4R, m=5+4R, m=6+4R, m=7+4R, m=8+4R, or m=9+4R, wherein R is the number of internal repeat modules comprised in said repeat domain.

In some embodiments, the designed ankyrin repeat domain of the invention has a total number of basic amino acid residues among the framework residues equal to or lower than x, wherein x=R, x=l+R, x=2+R, x=3+R, x=4+R, x=5+R, x=6+R, x=7+R, x=2R, x=1+2R, x=2+2R, x=3+2R, x=4+2R, x=5+2R, x=6+2R, x=7+2R, x=3R, x=1+3R, x=2+3R, x=3+3R, x=4+3R, x=5+3R, x=6+3R, or x=7+3R, wherein R is the number of internal repeat modules comprised in said repeat domain.

In other embodiments, the designed ankyrin repeat domain of the invention has a total number of Arg and Lys residues among the framework residues equal to or lower than y, wherein y=1+R, y=2-FR, y=3-FR, y=4-FR, y=5-FR, y=6-FR, y=2R, y=1+2R, y=2+2R, y=3+2R, y=4+2R, y=5+2R, or y=6+2R, wherein R is the number of internal repeat modules comprised in said repeat domain.

Preferably, designed ankyrin repeat domains of the invention bind specifically to a target. In preferred embodiments, the repeat domain of the invention binds to a target with a dissociation constant (K_{D}) of about 10 ⁻⁵ M or less, about 10⁻⁶ M or less, about 10⁻⁷ M or less, about 10⁻⁷ M or less, about 10⁻⁹ M or less, about 10 ⁻¹³ M or less, about 10⁻¹¹ M or less, about 10⁻¹² M or less, about 10⁻¹³ M or less, about 10⁻¹⁴ M or less.

In some embodiments, said target is HER2. Accordingly, in particular embodiments, the designed ankyrin repeat domains of the invention bind specifically to HER2. HER2, as used herein, relates to Human Epidermal Growth Factor Receptor 2, also known as Neu, ErbB-2, CD340 (cluster of differentiation 340) or p185. HER2 is a member of the epidermal growth factor receptor (EGFR/ErbB) family. HER2 is, in humans, encoded by ERBB2, a known proto-oncogene located at the long arm of human chromosome 17 (17q12).

HER2 has the UniProtKB/Swiss-Prot number P04626.

In one embodiment, the invention provides a designed ankyrin repeat domain comprising an N-terminal capping module, at least one internal repeat module and a C-terminal capping module, and wherein said repeat domain has at least one characteristic selected from the following list of characteristics:
(a) an isoelectric point (pI) equal to or lower than pH 4.6;
(b) among all amino acid residues comprised in said repeat domain, a percentage of basic amino acid residues equal to or lower than 12.0%;
(c) among all amino acid residues comprised in said repeat domain, a percentage of Arg and Lys residues equal to or lower than 8.0%;
(d) among the framework residues comprised in said repeat domain, a percentage of basic amino acid residues equal to or lower than 9.6%; and
(e) among the framework residues comprised in said repeat domain, a percentage of Arg and Lys residues equal to or lower than 6.7%.

In preferred embodiments, said repeat domain is linked to a drug moiety.

The invention encompasses embodiments in which said repeat domain has any combination of the above characteristics (a) to (e). Accordingly, in some embodiments, said repeat domain has at least characteristic (a), (b), (c), (d) or (e). In other embodiments, said repeat domain has at least characteristics (a) and (b), (a) and (c), (a) and (d), (a) and (e), (b) and (c), (b) and (d), (b) and (e), (c) and (d), (c) and (e) or (d) and (e). In other embodiments, said repeat domain has at least characteristics (a), (b) and (c); (a), (b) and (d); (a), (b) and (e); (a), (c) and (d); (a), (c) and (e); (a), (d) and (e); (b), (c) and (d); (b), (c) and (e); (b), (d) and (e) or (c), (d) and (e). In other embodiments, said repeat domain has at least characteristics (a), (b), (c) and (d); (a), (b), (c) and (e); (a), (b), (d) and (e); (a), (c), (d) and (e) or (b), (c), (d) and (e). In other embodiments, said repeat domain has characteristics (a), (b), (c), (d) and (e). In some preferred embodiments, said repeat domain has an isoelectric point (pI) is in a range between pH 3.0 and pH 4.6, preferably between pH 3.3 and pH 4.6, and more preferably between pH 3.50 and pH 4.53. In some preferred embodiments, said percentage in (b) is equal to or lower than 8.7%. In some most preferred embodiments, said percentage in (b) is equal to or lower than 8.06%. In some preferred embodiments, the percentage in (c) is equal to or lower than 4.7%. In some most preferred embodiments, said percentage in (c) is equal to or lower than 4.03%. In some preferred embodiments, said percentage in (d) is equal to or lower than 6.7%. In some most preferred embodiments, said percentage in (d) is equal to or lower than 6.1%. In some preferred embodiments, said percentage in (e) is equal to or lower than 5.7%. In some most preferred embodiments, said percentage in (e) is equal to or lower than 4.85%.

In some embodiments, said repeat domain has a total number of basic amino acid residues equal to or lower than n, wherein n=R, n=1+R, n=2+R, n=3+R, n=4+R, n=5+R, n=6+R, n=7+R, n=8+R, n=9+R, n=2R, n=1+2R, n=2+2R, n=3+2R, n=4+2R, n=5+2R, n=6+2R, n=7+2R, n=8+2R, n=9+2R, n=3R, n=1+3R, n=2+3R, n=3+3R, n=4+3R, n=5+3R, n=6+3R, n=7+3R, n=8+3R, n=9+3R, n=4R, n=1+4R, n=2+4R, n=3+4R, n=4+4R, n=5+4R, n=6+4R, n=7+4R, n=8+4R, n=9+4R, n=5R, n=1+5R, n=2+5R, n=3+5R, n=4+5R, n=5+5R, n=6+5R, n=7+5R, n=8+5R, n=9+5R, n=6R, n=1+6R, n=2+6R, n=3+6R, n=4+6R, n=5+6R, n=6+6R, n=7+6R, n=8+6R, or n=9+6R, wherein R is the number of internal repeat modules comprised in said repeat domain. In some more particular embodiments, n=4+5R. In such embodiments, “xR” means x multiplied by R.

In some embodiments, said repeat domain has a total number of Arg and Lys residues equal to or lower than m, wherein m=R, m=1+R, m=2+R, m=3+R, m=4+R, m=S+R, m=6+R, m=7+R, m=8+R, m=9+R, m=2R, m=1+2R, m=2+2R, m=3+2R, m=4+2R, m=5+2R, m=6+2R, m=7+2R, m=8+2R, m=9+2R, m=3R, m=1+3R, m=2+3R, m=3+3R, m=4+3R, m=5+3R, m=6+3R, m=7+3R, m=8+3R, m=9+3R, m=4R, m=1+4R, m=2+4R, m=3+4R, m=4+4R, m=5+4R, m=6+4R, m=7+4R, m=8+4R, or m=9+4R, wherein R is the number of internal repeat modules comprised in said repeat domain. In some more particular embodiments, m=5+2R.

In some embodiments, said repeat domain has a total number of basic amino acid residues among the framework residues equal to or lower than x, wherein x=R, x=1+R, x=2+R, x=3+R, x=4+R, x=S+R, x=6+R, x=7+R, x=2R, x=1+2R, x=2+2R, x=3+2R, x=4+2R, x=5+2R, x=6+2R, x=7+2R, x=3R, x=1+3R, x=2+3R, x=3+3R, x=4+3R, x=5+3R, x=6+3R, or x=7+3R, wherein R is the number of internal repeat modules comprised in said repeat domain.

In some embodiments, said repeat domain has a total number of Arg and Lys residues among the framework residues equal to or lower than y, wherein y=1+R, y=2+R, y=3+R, y=4+R, y=S+R, y=6+R, y=2R, y=1+2R, y=2+2R, y=3+2R, y=4+2R, y=5+2R, or y=6+2R, wherein R is the number of internal repeat modules comprised in said repeat domain.

In some embodiments, said repeat domain has no Arg residue and no Lys residue within the framework residue positions.

In some embodiments, said repeat domain comprises two internal repeat modules. In some embodiments, said repeat domain comprising two internal repeat modules may have a total number of basic amino acid residues equal to or lower than 1, 2, 3, 4, 5, 6, 7, 8, 9, 10, 11, 12, 13 or 14. Preferably said total number of basic residues is equal to or lower than 14. More preferably said total number of basic residues is equal to or lower than 10. In another particular embodiment, said repeat domain comprising two internal repeat modules may have a total number of Arg and Lys residues equal to or lower than 1, 2, 3, 4, 5, 6, 7, 8 or 9. Preferably said total number of Arg and Lys residues is equal to or lower than 9. More preferably, said total number of Arg and Lys residues is equal to or lower than 5. In another particular embodiment, said repeat domain comprising two internal repeat modules may have a total number of Arg and Lys residues among the framework residues equal to or lower than 1, 2, 3, 4, 5 or 6. Preferably said total number of Arg and Lys residues among the framework residues is equal to or lower than 6. More preferably, said total number of Arg and Lys residues among the framework residues is equal to or lower than 5.

In another embodiment, the invention provides a designed ankyrin repeat domain comprising an N-terminal capping module, at least one internal repeat module and a C-terminal capping module, wherein said repeat domain has at least one characteristic selected from the following list of characteristics:
(a) an isoelectric point (pI) equal to or lower than pH 4.07;
(b) among all amino acid residues comprised in said repeat domain, a percentage of basic amino acid residues equal to or lower than 7.0%; and
(c) among the framework residues comprised in said repeat domain, a percentage of basic amino acid residues equal to or lower than 6.1%.

The invention encompasses embodiments in which said repeat domain has any combination of the above characteristics (a) to (c). Accordingly, in some embodiments, said repeat domain has at least characteristic (a), (b) or (c). In other embodiments, said repeat domain has at least characteristics (a) and (b), (a) and (c), or (b) and (c). In other embodiments, said repeat domain has characteristics (a), (b) and (c).

In some embodiments, said repeat domain binds to a target with a dissociation constant (K_{D}) of about 10⁻⁵ M or less, about 10⁻⁶ M or less, about 10⁻⁷ M or less, about 10⁻⁸ M or less, about 10⁻⁹ M or less, about 10⁻¹⁹ M or less, about 10⁻¹¹ M or less, about 10⁻¹² M or less, about 10⁻¹³ M or less, or about 10⁻¹⁴ M or less. In a preferred embodiment, said repeat domain binds to a target with a dissociation constant (K_{D}) lower than 10⁻⁷.

The charge characteristics of the designed ankyrin repeat domain according to the invention can alternatively or in addition be defined by a ratio between the sum of Arg (R) and Lys (K) residues over the sum of Asp (D) and Glu (E) residues comprised in said designed ankyrin repeat domain (also referred to as KR/DE ratio). The KR/DE ratio can be computed based on the amino acid sequences of the designed ankyrin repeat domains of the invention.

Accordingly, in some embodiments, the designed ankyrin repeat domain of the invention has a KR/DE ratio equal to or lower than 0.44, equal to or lower than 0.42, equal to or lower than 0.40, equal to or lower than 0.38, equal to or lower than 0.36, equal to or lower than 0.34, equal to or lower than 0.32, equal to or lower than 0.30, equal to or lower than 0.28, equal to or lower than 0.26, equal to or lower than 0.24, equal to or lower than 0.22, equal to or lower than 0.20, equal to or lower than 0.18, equal to or lower than 0.16, equal to or lower than 0.14, equal to or lower than 0.12, equal to or lower than 0.10, or equal to or lower than 0.08. Preferably, said KR/DE ratio is equal to or lower than 0.3.

Unless specified otherwise, the KR/DE ratio is determined by considering all residues comprised in the repeat domain. In other embodiments, the KR/DE ratio is computed among the framework residues of the repeat domain only. Framework residues are defined in Table 4 as the residues in repeat modules that correspond to specific positions within the repeat modules of reference sequences. Accordingly, in some embodiments, the designed ankyrin repeat domain of the invention has a KR/DE ratio among the framework residues equal to or lower than 0.36, equal to or lower than 0.34, equal to or lower than 0.32, equal to or lower than 0.30, equal to or lower than 0.28, equal to or lower than 0.26, equal to or lower than 0.24, equal to or lower than 0.22, equal to or lower than 0.20, equal to or lower than 0.18, equal to or lower than 0.16, equal to or lower than 0.14, equal to or lower than 0.12, equal to or lower than 0.10, or equal to or lower than 0.08. Preferably, said KR/DE ratio among the framework residues is equal to or lower than 0.25.

In further embodiments, the KR/DE ratio is computed among all residues comprised in the N-terminal capping module only. Accordingly, in some embodiments, the designed ankyrin repeat domain of the invention has a KR/DE ratio among all residues of the N-terminal capping module equal to or lower than 0.66, equal to or lower than 0.64, equal to or lower than 0.62, equal to or lower than 0.60, equal to or lower than 0.58, equal to or lower than 0.56, equal to or lower than 0.54, equal to or lower than 0.52, equal to or lower than 0.50, equal to or lower than 0.48, equal to or lower than 0.46, equal to or lower than 0.44, equal to or lower than 0.42, equal to or lower than 0.40, equal to or lower than 0.38, equal to or lower than 0.36, equal to or lower than 0.34, equal to or lower than 0.32, equal to or lower than 0.30, equal to or lower than 0.28, equal to or lower than 0.26, equal to or lower than 0.24, equal to or lower than 0.22, equal to or lower than 0.20, equal to or lower than 0.18, equal to or lower than 0.16, equal to or lower than 0.14, equal to or lower than 0.12, equal to or lower than 0.10, equal to or lower than 0.08, equal to or lower than 0.06, equal to or lower than 0.04, or equal to or lower than 0.02. Preferably said KR/DE ratio among all residues of the N-terminal capping module is equal to or lower than 0.5.

In some embodiments, the designed ankyrin repeat domain of the invention comprises one or more internal repeat modules, wherein each internal repeat module independently comprises a sequence selected from the group consisting of (1) SEQ ID NOs: 131 to 142 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 131 to 142 are substituted by another amino acid.

In one embodiment, the designed ankyrin repeat domain of the invention comprises one or more internal repeat modules, wherein each internal repeat module independently comprises a sequence selected from the group consisting of (1) SEQ ID NOs: 131, 135 and 139 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 131, 135 and 139 are substituted by another amino acid.

In another embodiment, the designed ankyrin repeat domain of the invention comprises one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid.

Alternatively or in addition to the above defined internal repeat modules, in some embodiments, the designed ankyrin repeat domain of the invention comprises an N-terminal capping module having a sequence selected from the group consisting of (1) SEQ ID NOs: 25 and 115 to 122 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 25 and 115 to 122 are substituted by another amino acid.

In one embodiment, said N-terminal capping module comprises a sequence selected from the group consisting of (1) SEQ ID NOs: 115 and 121 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 115 and 121 are substituted by another amino acid.

In another embodiment, said N-terminal capping module comprises a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid.

Alternatively or in addition to the above defined internal repeat modules and/or N-terminal capping modules, in some embodiments, the designed ankyrin repeat domain of the invention comprises a C-terminal capping module having a sequence selected from the group consisting of (1) SEQ ID NOs: 123 to 130 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 to 130 are substituted by another amino acid.

In one embodiment, said C-terminal capping module comprises a sequence selected from the group consisting of (1) SEQ ID NOs: 123 and 126 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 and 126 are substituted by another amino acid.

In another embodiment, said C-terminal capping module comprises a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In a particular embodiment, the designed ankyrin repeat domain of the invention comprises:
(a) an N-terminal capping module having a sequence selected from the group consisting of (1) SEQ ID NOs: 115 and 121 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 115 and 121 are substituted by another amino acid, and (b) a C-terminal capping module having a sequence selected from the group consisting of (1) SEQ ID NOs:

123 and 126 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 and 126 are substituted by another amino acid.

In another embodiment, the designed ankyrin repeat domain of the invention comprises:
(a) an N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid, and
(b) a C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In a particular embodiment, the designed ankyrin repeat domain of the invention comprises:
(a) an N-terminal capping module having a sequence selected from the group consisting of (1) SEQ ID NOs: 115 and 121 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 115 and 121 are substituted by another amino acid, and
(b) one or more internal repeat modules, wherein each internal repeat module independently comprises a sequence selected from the group consisting of (1) SEQ ID NOs: 131, 135 and 139 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 131, 135 and 139 are substituted by another amino acid.

In another embodiment, the designed ankyrin repeat domain of the invention comprises:
(a) an N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid, and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid.

In a particular embodiment, the designed ankyrin repeat domain of the invention comprises:
(a) a C-terminal capping module having a sequence selected from the group consisting of (1) SEQ ID NOs: 123 and 126 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 and 126 are substituted by another amino acid, and
(b) one or more internal repeat modules, wherein each internal repeat module independently comprises a sequence selected from the group consisting of (1) SEQ ID NOs: 131, 135 and 139 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 131, 135 and 139 are substituted by another amino acid.

In another embodiment, the designed ankyrin repeat domain of the invention comprises:
(a) a C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid, and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid.

In a particular embodiment, the designed ankyrin repeat domain of the invention comprises:
(a) an N-terminal capping module having a sequence selected from the group consisting of (1) SEQ ID NOs: 115 and 121 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 115 and 121 are substituted by another amino acid; and
(b) one or more internal repeat modules, wherein each internal repeat module independently comprises a sequence selected from the group consisting of (1) SEQ ID NOs: 131, 135 and 139 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 131, 135 and 139 are substituted by another amino acid; and
(c) a C-terminal capping module having a sequence selected from the group consisting of (1) SEQ ID NOs: 123 and 126 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 and 126 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid; and
(c) a C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 9 framework residues in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 9 framework residues in SEQ ID NO: 131 are substituted by another amino acid; and
(c) a C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO:

115 and (2) sequences in which up to 5 framework residues in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 5 framework residues in SEQ ID NO: 131 are substituted by another amino acid; and
(c) a C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 3 framework residues in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 3 framework residues in SEQ ID NO: 131 are substituted by another amino acid; and
(c) a C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 9 framework residues in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid; and
(c) a C-terminal capping module having a sequence selected from the group consisting of (1) SEQ ID NO: 123 and (2) sequences in which up to 9 framework residues in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 5 framework residues in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid; and
(c) a C-terminal capping module having a sequence selected from the group consisting of (1) SEQ ID NO: 123 and (2) sequences in which up to 5 framework residues in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 3 framework residues in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid; and
(c) a C-terminal capping module having a sequence selected from the group consisting of (1) SEQ ID NO: 123 and (2) sequences in which up to 3 framework residues in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 2 framework residues in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid; and
(c) a C-terminal capping module having a sequence selected from the group consisting of (1) SEQ ID NO: 123 and (2) sequences in which up to 2 framework residues in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 1 framework residue in SEQ ID NO: 115 is substituted by another amino acid; and
(b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid; and
(c) a C-terminal capping module having a sequence selected from the group consisting of (1) SEQ ID NO: 123 and (2) sequences in which up to 1 framework residue in SEQ ID NO: 123 is substituted by another amino acid.

In one embodiment, the designed ankyrin repeat domain of the invention comprises:
(a) an N-terminal capping module having the sequence of SEQ ID NO: 115; and (b) one or more internal repeat modules, wherein each internal repeat module independently comprises a sequence selected from the group consisting of (1) SEQ ID NO: 131 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 131 are substituted by another amino acid; and
(c) a C-terminal capping module having the sequence of SEQ ID NO: 123.

In one embodiment, the designed ankyrin repeat domain of the invention comprises:
(a) an N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 9 framework residues in SEQ ID NO: 131 are substituted by another amino acid; and
(c) a C-terminal capping module having a sequence selected from the group consisting of (1) SEQ ID NO: 123 and (2) sequences in which up to 9 framework residues in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 5 framework residues in SEQ ID NO: 131 are substituted by another amino acid; and
(c) a C-terminal capping module having a sequence selected from the group consisting of (1) SEQ ID NO: 123 and (2) sequences in which up to 5 framework residues in SEQ ID NO: 123 are substituted by another amino acid.

In one embodiment, the designed ankyrin repeat domain of the invention comprises:
(a) an N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid; and
(b) one or more internal repeat modules, wherein each internal repeat module independently comprises a sequence selected from the group consisting of (1) SEQ ID NO: 131 and (2) sequences in which up to 3 framework residues in SEQ ID NO: 131 are substituted by another amino acid; and
(c) a C-terminal capping module having a sequence selected from the group consisting of (1) SEQ ID NO: 123 and (2) sequences in which up to 3 framework residues in SEQ ID NO: 123 are substituted by another amino acid.

In further embodiments, the designed ankyrin repeat domain of the invention comprises one or more internal repeat module independently comprises a sequence selected from the group consisting of (1) SEQ ID NO: 31 and (2) sequences in which up to 5, 4, 3, 2 or 1 framework residues other than positions 1, 10, 13, 17, 19, 21, 22 and 26 in SEQ ID NO: 31 are substituted by another amino acid, and/or said N-terminal capping module comprises a sequence selected from the group consisting of (1) SEQ ID NO: 19 and (2) sequences in which up to 5, 4, 3, 2 or 1 framework residues other than positions 5, 17, 20 and 23 in SEQ ID NOs: 19 are substituted by another amino acid, and/or said C-terminal capping module comprises a sequence selected from the group consisting of (1) SEQ ID NO: 40 and (2) sequences in which up to 5, 4, 3, 2 or 1 framework residues other than positions 10, 11, 17, 18, 19, 22 and 26 in SEQ ID NO: 40 are substituted by another amino acid.

In particular embodiments, the designed ankyrin repeat domain of the invention comprises a sequence selected from SEQ ID NOs: 19 to 25, 57 to 60, 65 to 81, 31 to 35, 61 to 62, 92 to 94, 40 to 42, 63 to 64 and 82 to 91.

In particular embodiments, each internal repeat module comprised in the designed ankyrin repeat domain of the invention may independently comprise a sequence selected from SEQ ID NOs: 31 to 35, 61 to 62 and 92 to 94. Alternatively or in addition, the N-terminal capping module comprised in the designed ankyrin repeat domain of the invention may comprise a sequence selected from SEQ ID NOs: 19 to 25, 57 to 60 and 65 to 81. Alternatively or in addition, the C-terminal capping module comprised in the ankyrin repeat domain of the invention may comprise a sequence selected from SEQ ID NOs: 40 to 42, 63 to 64 and 82 to 91.

In particular embodiments, each internal repeat module comprised in the designed ankyrin repeat domain of the invention independently comprises a sequence selected from SEQ ID NOs: 31 to 35, 61 to 62, 92 to 94 and 131 to 142. Alternatively or in addition, the N-terminal capping module comprised in the designed ankyrin repeat domain of the invention comprises a sequence selected from SEQ ID NOs: 19 to 25, 57 to 60, 65 to 81 and 115 to 122. Alternatively or in addition, the C-terminal capping module comprised in the ankyrin repeat domain of the invention comprises a sequence selected from SEQ ID NOs: 40 to 42, 63 to 64, 82 to 91 and 123 to 130.

In further embodiments, each internal repeat module comprised in the designed ankyrin repeat domain of the invention independently comprises a sequence selected from the group consisting of (1) SEQ ID NOs: 31 to 35, 61 to 62 and 92 to 94 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 31 to 35, 61 to 62 and 92 to 94 are substituted by another amino acid. Alternatively or in addition, the N-terminal capping module comprised in the designed ankyrin repeat domain of the invention comprises a sequence selected from the group consisting of (1) SEQ ID NOs: 19 to 25, 57 to 60 and 65 to 81 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 19 to 25, 57 to 60 and 65 to 81 are substituted by another amino acid. Alternatively or in addition, the C-terminal capping module comprised in the designed ankyrin repeat domain of the invention comprises a sequence selected from the group consisting of (1) SEQ ID NOs: 40 to 42, 63 to 64 and 82 to 91 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 40 to 42, 63 to 64 and 82 to 91 are substituted by another amino acid.

In particular embodiments, each internal repeat module comprised in the designed ankyrin repeat domain of the invention independently comprises a sequence selected from the group consisting of (1) SEQ ID NOs: 31 to 32 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 31 to 32 are substituted by another amino acid. Alternatively or in addition, the N-terminal capping module comprised in the designed ankyrin repeat domain of the invention comprises a sequence selected from the group consisting of (1) SEQ ID NOs: 19 to 20 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 19 to 20 are substituted by another amino acid.

Alternatively or in addition, the C-terminal capping module comprised in the designed ankyrin repeat domain of the invention comprises a sequence selected from the group consisting of (1) SEQ ID NOs: 40 to 41 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 40 to 41 are substituted by another amino acid.

In further embodiments, each internal repeat module comprised in the designed ankyrin repeat domain of the invention may independently comprise a sequence selected from the group consisting of (1) SEQ ID NOs: 26 to 30 and (2) sequences in which 1, 2, 3, 4, 5, 6, 7, 8 or 9 amino acids in any of SEQ ID NOs: 26 to 30 are substituted by another amino acid. Alternatively or in addition, the N-terminal capping module comprised in the designed ankyrin repeat domain of the invention may comprise a sequence selected from the group consisting of (1) SEQ ID NOs: 15 to 18 and (2) sequences in which 1, 2, 3, 4, 5, 6, 7, 8 or 9 amino acids in any of SEQ ID NOs: 15 to 18 are substituted by another amino acid. Alternatively or in addition, the C-terminal capping module comprised in the designed ankyrin repeat domain of the invention may comprise a sequence selected from the group consisting of (1) SEQ ID NOs: 36 to 39 and (2) sequences in which 1, 2, 3, 4, 5, 6, 7, 8 or 9 amino acids in any of SEQ ID NOs: 36 to 39 are substituted by another amino acid.

In further embodiments, each internal repeat module comprised in the designed ankyrin repeat domain of the invention may also independently comprise a sequence selected from the group consisting of (1) SEQ ID NOs: 46 to 52 and (2) sequences in which 1, 2, 3, 4, 5, 6, 7, 8 or 9 amino acids in any of SEQ ID NOs: 46 to 52 are substituted by another amino acid. Alternatively or in addition, the N-terminal capping module comprised in the designed ankyrin repeat domain of the invention may also comprise a sequence selected from the group consisting of (1) SEQ ID NOs: 43 to 45 and (2) sequences in which 1, 2, 3, 4, 5, 6, 7, 8 or 9 amino acids in any of SEQ ID NOs: 43 to 45 are substituted by another amino acid. Alternatively or in addition, the C-terminal capping module comprised in the ankyrin repeat domain of the invention may also comprise a sequence selected from the group consisting of (1) SEQ ID NOs: 53 to 56 and (2) sequences in which 1, 2, 3, 4, 5, 6, 7, 8 or 9 amino acids in any of SEQ ID NOs: 53 to 56 are substituted by another amino acid.

In further embodiments, each internal repeat module comprised in the designed ankyrin repeat domain of the invention independently comprises a sequence selected from the group consisting of (1) SEQ ID NOs: 26 to 30 and 46 to 52, and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 26 to 30 and 46 to 52 are substituted by another amino acid. Alternatively or in addition, the N-terminal capping module comprised in the designed ankyrin repeat domain of the invention comprises a sequence selected from the group consisting of (1) SEQ ID NOs: 15 to 18, 43 to 45 and 99 to 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 15 to 18, 43 to 45 and 99 to 101 are substituted by another amino acid. Alternatively or in addition, the C-terminal capping module comprised in the designed ankyrin repeat domain of the invention comprises a sequence selected from the group consisting of (1) SEQ ID NOs: 36 to 39, 53 to 56 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 36 to 39, 53 to 56 and 102 to 103 are substituted by another amino acid.

In further embodiments, each internal repeat module comprised in the designed ankyrin repeat domain of the invention independently comprises a sequence selected from the group consisting of (1) SEQ ID NOs: 26 to 30 and 47 to 48, and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 26 to 30 and 47 to 48 are substituted by another amino acid. Alternatively or in addition, the N-terminal capping module comprised in the designed ankyrin repeat domain of the invention comprises a sequence selected from the group consisting of (1) SEQ ID NOs: 15 to 18, 43 and 99 to 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 15 to 18, 43 and 99 to 101 are substituted by another amino acid. Alternatively or in addition, the C-terminal capping module comprised in the designed ankyrin repeat domain of the invention comprises a sequence selected from the group consisting of (1) SEQ ID NOs: 36 to 39, 54 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 36 to 39, 54 and 102 to 103 are substituted by another amino acid.

In further embodiments, each internal repeat module comprised in the designed ankyrin repeat domain of the invention independently comprises a sequence selected from the group consisting of (1) SEQ ID NOs: 47 to 48, and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 47 to 48 are substituted by another amino acid. Alternatively or in addition, the N-terminal capping module comprised in the designed ankyrin repeat domain of the invention comprises a sequence selected from the group consisting of (1) SEQ ID NOs: 43, 100, 99 and 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 43, 100, 99 and 101 are substituted by another amino acid. Alternatively or in addition, the C-terminal capping module comprised in the designed ankyrin repeat domain of the invention comprises a sequence selected from the group consisting of (1) SEQ ID NOs: 54 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 54 and 102 to 103 are substituted by another amino acid.

In some embodiments, each internal repeat module comprised in the designed ankyrin repeat domain of the invention comprises one or more residues selected from the group consisting of (i) Gln in position 1 and (ii) Glu in position 26, numbered relative to SEQ ID NO: 48.

In some embodiments, the N-terminal capping module comprised in the designed ankyrin repeat domain of the invention comprises one or more residues selected from the group consisting of (i) Thr in position 4, (ii) Gln in position 5, (iii) Leu, Val, Ile, Ala or Met in position 15, and (iv) Gln or Ala in position 23, numbered relative to SEQ ID NO: 43.

In some embodiments, the C-terminal capping module comprised in the designed ankyrin repeat domain of the invention comprises one or more residues selected from the group consisting of (i) Thr or Asn in position 3, (ii) Ser in position 4, (iii) Thr or Ser in position 6, (iv) Asn in position 17, (v) Glu in position 18, and (vi) Asp in position 19, numbered relative to SEQ ID NO: 54.

In some embodiments, the designed ankyrin repeat domain of the invention comprises (1) an N-terminal capping module comprising one or more residues selected from the group consisting of (i) Thr in position 4, (ii) Gln in position 5, (iii) Leu, Val, Ile, Ala or Met in position 15, and (iv) Gln or Ala in position 23, numbered relative to SEQ ID NO: 43, and/or (2) one or more internal repeat modules, wherein each internal repeat module independently comprises one or more residues selected from the group consisting of (i) Gln in position 1 and (ii) Glu in position 26, numbered relative to SEQ ID NO: 48, and/or (3) a C-terminal capping module comprising one or more residues selected from the group consisting of (i) Thr or Asn in position 3, (ii) Ser in position 4, (iii) Thr or Ser in position 6, (iv) Asn in position 17, (v) Glu in position 18, and (vi) Asp in position 19, numbered relative to SEQ ID NO: 54.

Accordingly, in one exemplary embodiment, the designed ankyrin repeat domain of the invention comprises (1) an N-terminal capping module comprising Thr in position 4, Gln in position 5, Leu in position 15 and Ala in position 23, numbered relative to SEQ ID NO: 43, (2) two internal repeat modules, each comprising a Glu in position 26, numbered relative to SEQ ID NO: 48, and (3) a C-terminal capping module comprising Thr in position 3, Ser in position 4, Ser in position 6, Asn in position 17, Glu in position 18 and Asp in position 19, numbered relative to SEQ ID NO: 54.

In another exemplary embodiment, the designed ankyrin repeat domain of the invention comprises (1) an N-terminal capping module comprising Thr in position 4, Gln in position 5, Leu in position 15 and Gln in position 23, numbered relative to SEQ ID NO: 43, (2) two internal repeat modules, each comprising Gln in position 1 and Glu in position 26, numbered relative to SEQ ID NO: 48, and (3) a C-terminal capping module comprising Asn in position 17, Glu in position 18 and Asp in position 19, numbered relative to SEQ ID NO: 54.

In further embodiments, the designed ankyrin repeat domain of the invention comprises a sequence selected from the group consisting of (1) SEQ ID NOs: 104 to 114 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity among the framework residues of any one of SEQ ID NOs: 104 to 114. Preferably, the designed ankyrin repeat domain of the invention comprises a sequence selected from the group consisting of (1) SEQ ID NOs: 104 and 112 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity among the framework residues of any one of SEQ ID NOs: 104 and 112. More preferably, the designed ankyrin repeat domain of the invention comprises a sequence selected from the group consisting of (1) SEQ ID NO: 104 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity among the framework residues of SEQ ID NO: 104 are substituted by another amino acid.

In a particular embodiment, the designed ankyrin repeat domain of the invention comprises a sequence selected from the group consisting of (1) SEQ ID NOs: 2 to 5, 8 to 10, 12 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 2 to 5, 8 to 10, 12 and 95 to 98. Thus, in a more particular embodiment, said designed repeat domain comprises an amino acid sequence selected from the group consisting of (1) SEQ ID NOs: 2 to 5, 8 to 10, 12 and 95 to 98 and (2) sequences with at least 80% amino acid sequence identity with any one of SEQ ID NOs: 2 to 5, 8 to 10, 12 and 95 to 98.

In a particular embodiment, the designed ankyrin repeat domain of the invention comprises a sequence selected from the group consisting of (1) SEQ ID NOs: 8 to 10, 12 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 8 to 10, 12 and 95 to 98. Thus, in a more particular embodiment, said designed repeat domain comprises an amino acid sequence selected from the group consisting of (1) SEQ ID NOs: 8 to 10, 12 and 95 to 98 and (2) sequences with at least 80% amino acid sequence identity with any one of SEQ ID NOs: 8 to 10, 12 and 95 to 98.

In a particular embodiment, the designed ankyrin repeat domain of the invention comprises a sequence selected from the group consisting of (1) SEQ ID NOs: 8 to 10 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ

ID NOs: 8 to 10 and 95 to 98. Thus, in a more particular embodiment, said designed repeat domain comprises an amino acid sequence selected from the group consisting of (1) SEQ ID NOs: 8 to 10 and 95 to 98 and (2) sequences with at least 80% amino acid sequence identity with any one of SEQ ID NOs: 8 to and 95 to 98.

In a particular embodiment, the designed ankyrin repeat domain of the invention comprises a sequence selected from the group consisting of (1) SEQ ID NOs: 8 to 9 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 8 to 9 and 95 to 98. Thus, in a more particular embodiment, said designed repeat domain comprises an amino acid sequence selected from the group consisting of (1) SEQ ID NOs: 8 to 9 and 95 to 98 and (2) sequences with at least 80% amino acid sequence identity with any one of SEQ ID NOs: 8 to 9 and 95 to 98.

In a particular embodiment, the designed ankyrin repeat domain of the invention comprises a sequence selected from the group consisting of (1) SEQ ID NOs: 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 95 to 98. Thus, in a more particular embodiment, said designed repeat domain comprises an amino acid sequence selected from the group consisting of (1) SEQ ID NOs: 95 to 98 and (2) sequences with at least 80% amino acid sequence identity with any one of SEQ ID NOs: 95 to 98.

In a particular embodiment, the designed ankyrin repeat domain of the invention comprises a sequence selected from the group consisting of (1) SEQ ID NO: 12 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with SEQ ID NO: 12. Thus, in a more particular embodiment, said designed repeat domain comprises an amino acid sequence selected from the group consisting of (1) SEQ ID NO: 12 and (2) sequences with at least 80% amino acid sequence identity with SEQ ID NO: 12.

In a particular embodiment, the designed ankyrin repeat domain of the invention comprises a sequence selected from the group consisting of (1) SEQ ID NOs: 2 to 5 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 2 to 5. Thus, in a more particular embodiment, said designed repeat domain comprises an amino acid sequence selected from the group consisting of (1) SEQ ID NOs: 2 to 5 and (2) sequences with at least 80% amino acid sequence identity with any one of SEQ ID NOs: 2 to 5.

In a particular embodiment, the designed ankyrin repeat domain of the invention comprises a sequence selected from the group consisting of (1) SEQ ID NOs: 2, 3 and 5 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs:

2, 3 and 5. Thus, in a more particular embodiment, said designed repeat domain comprises an amino acid sequence selected from the group consisting of (1) SEQ ID NOs: 2, 3 and 5 and (2) sequences with at least 80% amino acid sequence identity with any one of SEQ ID NOs: 2, 3 and 5.

In further embodiments, the designed ankyrin repeat domain of the invention comprises one or more residues selected from the group consisting of (i) Thr in position 4, (ii) Gln in position 5, (iii) Leu, Val, Ile, Ala or Met in position 15, (iv) Gln or Ala in position 23, (v) Gln in position 31, (vi) Glu in position 56, (vii) Gln in position 64, (viii) Glu in position 89, (ix) Thr or Asn in position 99, (x) Ser in position 100, (xi) Thr or Ser in position 102, (xii) Asn in position 113, (xiii) Glu in position 114, and (xiv) Asp in position 115, numbered relative to SEQ ID NO: 6.

Accordingly, in an exemplary embodiment, the designed ankyrin repeat domain of the invention comprises Thr in position 4, Gln in position 5, Leu in position 15, Ala in position 23, Glu in position 56, Glu in position 89, Thr in position 99, Ser in position 100, Ser in position 102, Asn in position 113, Glu in position 114 and Asp in position 115, numbered relative to SEQ ID NO: 6.

In another exemplary embodiment, the designed ankyrin repeat domain of the invention comprises Thr in position 4, Gln in position 5, Leu in position 15, Gln in position 23, Gln in position 31, Glu in position 56, Gln in position 64, Glu in position 89, Asn in position 113, Glu in position 114 and Asp in position 115, numbered relative to SEQ ID NO: 6.

Furthermore, the sequence of any repeat domain of the present invention may optionally comprise at its N-terminus, a G, an S, or a GS. Furthermore, the sequence of any repeat domain of the present invention may optionally have A at the second last position substituted with L and/or A at the last position substituted with N.

In some embodiments, the designed ankyrin repeat domain of the invention is linked to a drug moiety. In some embodiments, the drug moiety is linked covalently or non-covalently to the designed ankyrin repeat domain of the invention. In a more particular embodiment, the drug moiety is linked to the designed ankyrin repeat domain of the invention by a chelator. Appropriate chelators may be selected depending on the application of said repeat domain of the invention. In a particular embodiment, said chelator is DTPA. In a more particular embodiment, said drug moiety is a radionuclide. The choice of said radionuclide may depend on the intended application of the designed ankyrin repeat domain of the invention (e.g. diagnostic vs. therapeutic).

In a particular embodiment, said drug moiety is a therapeutic moiety. In one embodiment, said therapeutic moiety is a toxin. In one embodiment, said therapeutic moiety is a radionuclide. In a more particular embodiment, said radionuclide is indium-111.

In another particular embodiment, said drug moiety is a diagnostic moiety. In a more particular embodiment, said diagnostic moiety is a fluorophore, a chromophore, an imaging agent or a radionuclide.

In another aspect, the invention provides a designed ankyrin repeat domain obtained or obtainable by the method according to the invention, described hereinbelow.

Generation Method According to the Invention

Designed ankyrin repeat domains having a reduced pI and/or reduced number of basic amino acids according to the invention may be generated by starting from the sequence of a conventional designed ankyrin repeat domain which does not possess a pI and/or a number of basic amino acids as low as or lower than the threshold values described herein.

Substitution of basic amino acids residues with neutral or acidic amino acid residues are most important for the generation of designed ankyrin repeat domains of the invention having improved pharmacokinetic properties. However, given that substitution of neutral amino acid residues with acidic amino acid residues in a protein typically also induces a decrease in isoelectric point (pI), such substitutions may be performed in a complementary manner to substitution of basic amino acid residues with neutral or acidic amino acid residues, where appropriate.

Accordingly, in one aspect, the invention provides a method of generating a modified ankyrin repeat domain, the method comprising steps (a) and (b),
wherein step (a) is providing an ankyrin repeat domain which has an isoelectric point (pI) higher than pH 4.07, and/or among all amino acid residues comprised in the repeat domain, a percentage of basic amino acid residues higher than 7.0%, and/or among the framework residues comprised in the repeat domain, a percentage of basic amino acid residues higher than 6.1%; and
wherein step (b) is (1) substituting at least one basic amino acid residue of the repeat domain of step (a) with a neutral or acidic amino acid residue; and/or
(2) substituting at least one neutral amino acid residue of the repeat domain of step (a) with an acidic amino acid;
   wherein said modified ankyrin repeat domain has at least one characteristic selected from the following list of characteristics:
(i) an isoelectric point (pI) equal to or lower than pH 4.07;
(ii) among all amino acid residues comprised in the repeat domain, a percentage of basic amino acid residues equal to or lower than 7.0%; and
(iii) among the framework residues comprised in the repeat domain, a percentage of basic amino acid residues equal to or lower than 6.1%.

For clarity, the pI and percentage numerical values in step (a) of said method preferably correlate with the pI and percentage numerical values in the characteristics (i) to (iii), such that if in some embodiment another numerical value is selected in one of the characteristics (i) to (iii), said numerical value may apply to the corresponding feature of the repeat domain of step (a). Thus, if one of the characteristics (i) to (iii) involves a feature that is “equal to or lower than” X, then the corresponding feature of step (a) should be “higher than” X.

Further for clarity, at least one of the properties listed for the ankyrin repeat domain provided in step (a) is modified by step (b) in such a way that the corresponding characteristic of (i) to (iii) is achieved.

In one particular embodiment, said method according to the invention comprises steps (a) and (b),
wherein step (a) is providing an ankyrin repeat domain which has an isoelectric point (pI) higher than pH 4.07, and/or among all amino acid residues comprised in the repeat domain, a percentage of basic amino acid residues higher than 7.0%, and/or among the framework residues comprised in the repeat domain, a percentage of basic amino acid residues higher than 6.1%; and
wherein step (b) is (1) substituting at least one basic amino acid residue of the repeat domain of step (a) with a neutral or acidic amino acid residue; and
(2) substituting at least one neutral amino acid residue of the repeat domain of step (a) with an acidic amino acid;
   wherein said modified ankyrin repeat domain has at least one characteristic selected from the following list of characteristics:
(i) an isoelectric point (pI) equal to or lower than pH 4.07;
(ii) among all amino acid residues comprised in the repeat domain, a percentage of basic amino acid residues equal to or lower than 7.0%; and
(iii) among the framework residues comprised in the repeat domain, a percentage of basic amino acid residues equal to or lower than 6.1%.

In another embodiment, said method according to the invention comprises steps (a) and (b),
wherein step (a) is providing an ankyrin repeat domain which has an isoelectric point (pI) higher than pH 4.07, and/or among all amino acid residues comprised in the repeat domain, a percentage of basic amino acid residues higher than 7.0%, and/or among the framework residues comprised in the repeat domain, a percentage of basic amino acid residues higher than 6.1%; and
wherein step (b) is substituting at least one basic amino acid residue of the repeat domain of step (a) with a neutral or acidic amino acid residue;
   wherein said modified ankyrin repeat domain has at least one characteristic selected from the following list of characteristics:
(i) an isoelectric point (pI) equal to or lower than pH 4.07;
(ii) among all amino acid residues comprised in the repeat domain, a percentage of basic amino acid residues equal to or lower than 7.0%; and
(iii) among the framework residues comprised in the repeat domain, a percentage of basic amino acid residues equal to or lower than 6.1%.

In another embodiment, said method according to the invention comprises steps (a) and (b),
wherein step (a) is providing an ankyrin repeat domain which has an isoelectric point (pI) higher than pH 4.07, and/or among all amino acid residues comprised in the repeat domain, a percentage of basic amino acid residues higher than 7.0%, and/or among the framework residues comprised in the repeat domain, a percentage of basic amino acid residues higher than 6.1%; and
wherein step (b) is substituting at least one neutral amino acid residue of the repeat domain of step (a) with an acidic amino acid;
   wherein said modified ankyrin repeat domain has at least one characteristic selected from the following list of characteristics:
(i) an isoelectric point (pI) equal to or lower than pH 4.07;
(ii) among all amino acid residues comprised in the repeat domain, a percentage of basic amino acid residues equal to or lower than 7.0%; and
(iii) among the framework residues comprised in the repeat domain, a percentage of basic amino acid residues equal to or lower than 6.1%.

The invention encompasses embodiments in which said modified repeat domain in the method of the invention has any combination of the above characteristics (i) to (iii). Accordingly, in some embodiments, said modified repeat domain has at least characteristic (i), (ii) or (iii). In other embodiments, said modified repeat domain has at least characteristics (i) and (ii), (i) and (iii), or (ii) and (iii). In other embodiments, said modified repeat domain has characteristics (i), (ii) and (iii).

In some preferred embodiments, said modified repeat domain has an isoelectric point (pI) is in a range between pH 3.0 and pH 4.07, preferably between pH 3.3 and pH 4.07, and more preferably between pH 3.50 and pH 4.07.

In one embodiment, said method according to the invention comprises steps (a) and (b),
wherein step (a) is providing an ankyrin repeat domain which has an isoelectric point (pI) higher than pH 4.07; and
wherein step (b) is (1) substituting at least one basic amino acid residue of the repeat domain of step (a) with a neutral or acidic amino acid residue; and/or
(2) substituting at least one neutral amino acid residue of the repeat domain of step (a) with an acidic amino acid;
   wherein said modified ankyrin repeat domain has an isoelectric point (pI) equal to or lower than pH 4.07.

In a more particular embodiment, said method according to the invention comprises steps (a) and (b),
wherein step (a) is providing an ankyrin repeat domain which has an isoelectric point (pI) higher than pH 4.07; and
wherein step (b) is (1) substituting at least one basic amino acid residue of the repeat domain of step (a) with a neutral or acidic amino acid residue; and
(2) substituting at least one neutral amino acid residue of the repeat domain of step (a) with an acidic amino acid;
   wherein said modified ankyrin repeat domain has an isoelectric point (pI) equal to or lower than pH 4.07.

In another particular embodiment, said method according to the invention comprises steps (a) and (b),
wherein step (a) is providing an ankyrin repeat domain which has an isoelectric point (pI) higher than pH 4.07; and
wherein step (b) is substituting at least one basic amino acid residue of the repeat domain of step (a) with a neutral or acidic amino acid residue;
   wherein said modified ankyrin repeat domain has an isoelectric point (pI) equal to or lower than pH 4.07.

In another particular embodiment, said method according to the invention comprises steps (a) and (b),
wherein step (a) is providing an ankyrin repeat domain which has an isoelectric point (pI) higher than pH 4.07; and
wherein step (b) is substituting at least one neutral amino acid residue of the repeat domain of step (a) with an acidic amino acid;
   wherein said modified ankyrin repeat domain has an isoelectric point (pI) equal to or lower than pH 4.07.

In one embodiment, said method according to the invention comprises steps (a) and (b),
wherein step (a) is providing an ankyrin repeat domain which has, among all amino acid residues comprised in the repeat domain, a percentage of basic amino acid residues higher than 7.0%; and
wherein step (b) comprises substituting at least one basic amino acid residue of the repeat domain of step (a) with a neutral or acidic amino acid residue;
   wherein said modified ankyrin repeat domain has, among all amino acid residues comprised in the repeat domain, a percentage of basic amino acid residues equal to or lower than 7.0%.

In a more particular embodiment, said method according to the invention comprises steps (a) and (b),
wherein step (a) is providing an ankyrin repeat domain which has, among all amino acid residues comprised in the repeat domain, a percentage of basic amino acid residues higher than 7.0%; and
wherein step (b) is (1) substituting at least one basic amino acid residue of the repeat domain of step (a) with a neutral or acidic amino acid residue; and
(2) substituting at least one neutral amino acid residue of the repeat domain of step (a) with an acidic amino acid;
   wherein said modified ankyrin repeat domain has, among all amino acid residues comprised in the repeat domain, a percentage of basic amino acid residues equal to or lower than 7.0%.

In another more particular embodiment, said method according to the invention comprises steps (a) and (b),
wherein step (a) is providing an ankyrin repeat domain which has, among all amino acid residues comprised in the repeat domain, a percentage of basic amino acid residues higher than 7.0%; and
wherein step (b) is substituting at least one basic amino acid residue of the repeat domain of step (a) with a neutral or acidic amino acid residue;
   wherein said modified ankyrin repeat domain has, among all amino acid residues comprised in the repeat domain, a percentage of basic amino acid residues equal to or lower than 7.0%.

In one embodiment, said method according to the invention comprises steps (a) and (b),
wherein step (a) is providing an ankyrin repeat domain which has, among the framework residues comprised in the repeat domain, a percentage of basic amino acid residues higher than 6.1%; and
wherein step (b) comprises substituting at least one basic amino acid residue among the framework residues of the repeat domain of step (a) with a neutral or acidic amino acid residue;
   wherein said modified ankyrin repeat domain has, among the framework residues comprised in the repeat domain, a percentage of basic amino acid residues equal to or lower than 6.1%.

In a more particular embodiment, said method according to the invention comprises steps (a) and (b),
wherein step (a) is providing an ankyrin repeat domain which has, among the framework residues comprised in the repeat domain, a percentage of basic amino acid residues higher than 6.1%; and
wherein step (b) is (1) substituting at least one basic amino acid residue among the framework residues of the repeat domain of step (a) with a neutral or acidic amino acid residue; and
(2) substituting at least one neutral amino acid residue of the repeat domain of step (a) with an acidic amino acid;
   wherein said modified ankyrin repeat domain has, among the framework residues comprised in the repeat domain, a percentage of basic amino acid residues equal to or lower than 6.1%.

In another more particular embodiment, said method according to the invention comprises steps (a) and (b),
wherein step (a) is providing an ankyrin repeat domain which has, among the framework residues comprised in the repeat domain, a percentage of basic amino acid residues higher than 6.1%; and
wherein step (b) is substituting at least one basic amino acid residue among the framework residues of the repeat domain of step (a) with a neutral or acidic amino acid residue;
   wherein said modified ankyrin repeat domain has, among the framework residues comprised in the repeat domain, a percentage of basic amino acid residues equal to or lower than 6.1%.

In other embodiments of said method according to the invention, the repeat domain in step (a) has a KR/DE ratio higher than 0.44 and the modified repeat domain has a KR/DE ratio equal to or lower than 0.44.

In other embodiments of said method according to the invention, the repeat domain in step (a) has a KR/DE ratio among the framework residues higher than 0.36 and the modified repeat domain has a KR/DE ratio equal to or lower than 0.36.

In other embodiments of said method according to the invention, the repeat domain in step (a) has a KR/DE ratio among all residues of the N-terminal capping module higher than 0.66 and the modified repeat domain has a KR/DE ratio among all residues of the N-terminal capping module equal to or lower than 0.66.

In other embodiments of said method according to the invention, the repeat domain in step (a) has a KR/DE ratio higher than 0.44 and the modified repeat domain has a KR/DE ratio equal to or lower than 0.44, and/or the repeat domain in step (a) has a KR/DE ratio among the framework residues higher than 0.36 and the modified repeat domain has a KR/DE ratio equal to or lower than 0.36, and/or the repeat domain in step (a) has a KR/DE ratio among all residues of the N-terminal capping module higher than 0.66 and the modified repeat domain has a KR/DE ratio among all residues of the N-terminal capping module equal to or lower than 0.66.

In another aspect, the invention provides a method of generating a modified ankyrin repeat domain linked to a drug moiety, the method comprising steps (a) and (b),
wherein step (a) is providing an ankyrin repeat domain which has an isoelectric point (pI) higher than pH 4.6, and/or among all amino acid residues comprised in the repeat domain, a percentage of basic amino acid residues higher than 12.0%, and/or among all amino acid residues comprised in the repeat domain, a percentage of Arg and Lys residues higher than 8.0%, and/or among the framework residues comprised in the repeat domain, a percentage of basic amino acid residues higher than 9.6%, and/or among the framework residues comprised in the repeat domain, a percentage of Arg and Lys residues higher than 6.7%; and
wherein step (b) is (1) substituting at least one basic amino acid residue of the repeat domain of step (a) with a neutral or acidic amino acid residue; and/or
(2) substituting at least one neutral amino acid residue of the repeat domain of step (a) with an acidic amino acid;
   wherein said modified repeat domain has at least one characteristic selected from the following list of characteristics:
(i) an isoelectric point (pI) equal to or lower than pH 4.6;
(ii) among all amino acid residues comprised in the repeat domain, a percentage of basic amino acid residues equal to or lower than 12.0%;
(iii) among all amino acid residues comprised in the repeat domain, a percentage of Arg and Lys residues equal to or lower than 8.0%;
(iv) among the framework residues comprised in the repeat domain, a percentage of basic amino acid residues equal to or lower than 9.6%; and
(v) among the framework residues comprised in the repeat domain, a percentage of Arg and Lys residues equal to or lower than 6.7%.

For clarity, the pI and percentage numerical values in step (a) of said method preferably correlate with the pI and percentage numerical values in the characteristics (i) to (v), such that if in some embodiment another numerical value is selected in one of the characteristics (i) to (v), said numerical value may apply to the corresponding feature of the repeat domain of step (a). Thus, if one of the characteristics (i) to (v) involves a feature that is “equal to or lower than” X, then the corresponding feature of step (a) should be “higher than” X.

Further for clarity, at least one of the properties listed for the ankyrin repeat domain provided in step (a) is modified by step (b) in such a way that the corresponding characteristic of (i) to (v) is achieved.

In one embodiment, said method according to the invention comprises steps (a) and (b),
wherein step (a) is providing an ankyrin repeat domain which has an isoelectric point (pI) higher than pH 4.6, and/or among all amino acid residues comprised in the repeat domain, a percentage of basic amino acid residues higher than 12.0%, and/or among all amino acid residues comprised in the repeat domain, a percentage of Arg and Lys residues higher than 8.0%, and/or among the framework residues comprised in the repeat domain, a percentage of basic amino acid residues higher than 9.6%, and/or among the framework residues comprised in the repeat domain, a percentage of Arg and Lys residues higher than 6.7%; and
wherein step (b) is (1) substituting at least one basic amino acid residue of the repeat domain of step (a) with a neutral or acidic amino acid residue; and
(2) substituting at least one neutral amino acid residue of the repeat domain of step (a) with an acidic amino acid;
   wherein said modified repeat domain has at least one characteristic selected from the following list of characteristics:
(i) an isoelectric point (pI) equal to or lower than pH 4.6;
(ii) among all amino acid residues comprised in the repeat domain, a percentage of basic amino acid residues equal to or lower than 12.0%;
(iii) among all amino acid residues comprised in the repeat domain, a percentage of Arg and Lys residues equal to or lower than 8.0%;
(iv) among the framework residues comprised in the repeat domain, a percentage of basic amino acid residues equal to or lower than 9.6%; and
(v) among the framework residues comprised in the repeat domain, a percentage of Arg and Lys residues equal to or lower than 6.7%.

In another embodiment, said method according to the invention comprises steps (a) and (b),
wherein step (a) is providing an ankyrin repeat domain which has an isoelectric point (pI) higher than pH 4.6, and/or among all amino acid residues comprised in the repeat domain, a percentage of basic amino acid residues higher than 12.0%, and/or among all amino acid residues comprised in the repeat domain, a percentage of Arg and Lys residues higher than 8.0%, and/or among the framework residues comprised in the repeat domain, a percentage of basic amino acid residues higher than 9.6%, and/or among the framework residues comprised in the repeat domain, a percentage of Arg and Lys residues higher than 6.7%; and
wherein step (b) is substituting at least one basic amino acid residue of the repeat domain of step (a) with a neutral or acidic amino acid residue;
   wherein said modified repeat domain has at least one characteristic selected from the following list of characteristics:
(i) an isoelectric point (pI) equal to or lower than pH 4.6;
(ii) among all amino acid residues comprised in the repeat domain, a percentage of basic amino acid residues equal to or lower than 12.0%;
(iii) among all amino acid residues comprised in the repeat domain, a percentage of Arg and Lys residues equal to or lower than 8.0%;
(iv) among the framework residues comprised in the repeat domain, a percentage of basic amino acid residues equal to or lower than 9.6%; and (v) among the framework residues comprised in the repeat domain, a percentage of Arg and Lys residues equal to or lower than 6.7%.

In a further embodiment, said method according to the invention comprises steps (a) and (b),
wherein step (a) is providing an ankyrin repeat domain which has an isoelectric point (pI) higher than pH 4.6, and/or among all amino acid residues comprised in the repeat domain, a percentage of basic amino acid residues higher than 12.0%, and/or among all amino acid residues comprised in the repeat domain, a percentage of Arg and Lys residues higher than 8.0%, and/or among the framework residues comprised in the repeat domain, a percentage of basic amino acid residues higher than 9.6%, and/or among the framework residues comprised in the repeat domain, a percentage of Arg and Lys residues higher than 6.7%; and
wherein step (b) is substituting at least one neutral amino acid residue of the repeat domain of step (a) with an acidic amino acid;
   wherein said modified repeat domain has at least one characteristic selected from the following list of characteristics:
(i) an isoelectric point (pI) equal to or lower than pH 4.6;
(ii) among all amino acid residues comprised in the repeat domain, a percentage of basic amino acid residues equal to or lower than 12.0%;
(iii) among all amino acid residues comprised in the repeat domain, a percentage of Arg and Lys residues equal to or lower than 8.0%;
(iv) among the framework residues comprised in the repeat domain, a percentage of basic amino acid residues equal to or lower than 9.6%; and
(v) among the framework residues comprised in the repeat domain, a percentage of Arg and Lys residues equal to or lower than 6.7%.

The invention encompasses embodiments in which said modified repeat domain in the method of the invention has any combination of the above characteristics (i) to (v). Accordingly, in some embodiments, said repeat domain has at least characteristic (i), (ii), (iii), (iv) or (v). In other embodiments, said repeat domain has at least characteristics (i) and (ii), (i) and (iii), (i) and (iv), (i) and (v), (ii) and (iii), (ii) and (iv), (ii) and (v), (iii) and (iv), (iii) and (v) or (iv) and (v). In other embodiments, said repeat domain has at least characteristics (i), (ii) and (iii); (i), (ii) and (iv); (i), (ii) and (v); (i), (iii) and (iv); (i), (iii) and (v); (i), (iv) and (v); (ii), (iii) and (iv); (ii), (iii) and (v); (ii), (iv) and (v) or (iii), (iv) and (v). In other embodiments, said repeat domain has at least characteristics (i), (ii), (iii) and (iv); (i), (ii), (iii) and (v); (i), (ii), (iv) and (v); (i), (iii), (iv) and (v) or (ii), (iii), (iv) and (v). In other embodiments, said modified repeat domain has characteristics (i), (ii), (iii), (iv) and (v).

In some preferred embodiments, said modified repeat domain has an isoelectric point (pI) is in a range between pH 3.0 and pH 4.6, preferably between pH 3.3 and pH 4.6, and more preferably between pH 3.50 and pH 4.53. In some preferred embodiments, said percentage in (ii) is equal to or lower than 8.7%. In some most preferred embodiments, the percentage in (ii) is equal to or lower than 8.06%. In some preferred embodiments, said percentage in (iii) is equal to or lower than 4.7%. In some most preferred embodiments, said percentage in (iii) is equal to or lower than 4.03%. In some preferred embodiments, said percentage in (iv) is equal to or lower than 6.7%. In some most preferred embodiments, said percentage in (iv) is equal to or lower than 6.1%. In some preferred embodiments, said percentage in (v) is equal to or lower than 5.7%. In some most preferred embodiments, said percentage in (v) is equal to or lower than 4.85%.

In one embodiment, said method according to the invention comprises steps (a) and (b),
wherein step (a) is providing an ankyrin repeat domain which has an isoelectric point (pI) higher than pH 4.6; and
wherein step (b) is (1) substituting at least one basic amino acid residue of the repeat domain of step (a) with a neutral or acidic amino acid residue; and/or (2) substituting at least one neutral amino acid residue of the repeat domain of step (a) with an acidic amino acid;
   wherein said modified repeat domain has an isoelectric point (pI) equal to or lower than pH 4.6.

In a more particular embodiment, said method according to the invention comprises steps (a) and (b),
wherein step (a) is providing an ankyrin repeat domain which has an isoelectric point (pI) higher than pH 4.6; and
wherein step (b) is (1) substituting at least one basic amino acid residue of the repeat domain of step (a) with a neutral or acidic amino acid residue; and
(2) substituting at least one neutral amino acid residue of the repeat domain of step (a) with an acidic amino acid;
   wherein said modified repeat domain has an isoelectric point (pI) equal to or lower than pH 4.6.

In another more particular embodiment, said method according to the invention comprises steps (a) and (b),
wherein step (a) is providing an ankyrin repeat domain which has an isoelectric point (pI) higher than pH 4.6; and
wherein step (b) is substituting at least one basic amino acid residue of the repeat domain of step (a) with a neutral or acidic amino acid residue;
   wherein said modified repeat domain has an isoelectric point (pI) equal to or lower than pH 4.6.

In another more particular embodiment, said method according to the invention comprises steps (a) and (b),
wherein step (a) is providing an ankyrin repeat domain which has an isoelectric point (pI) higher than pH 4.6; and
wherein step (b) is substituting at least one neutral amino acid residue of the repeat domain of step (a) with an acidic amino acid;
   wherein said modified repeat domain has an isoelectric point (pI) equal to or lower than pH 4.6.

In one embodiment, said method according to the invention comprises steps (a) and (b),
wherein step (a) is providing an ankyrin repeat domain which has, among all amino acid residues comprised in the repeat domain, a percentage of basic amino acid residues higher than 12.0%; and
wherein step (b) comprises substituting at least one basic amino acid residue of the repeat domain of step (a) with a neutral or acidic amino acid residue;
   wherein said modified repeat domain has, among all amino acid residues comprised in the repeat domain, a percentage of basic amino acid residues equal to or lower than 12.0%.

In a more particular embodiment, said method according to the invention comprises steps (a) and (b),
wherein step (a) is providing an ankyrin repeat domain which has, among all amino acid residues comprised in the repeat domain, a percentage of basic amino acid residues higher than 12.0%; and
wherein step (b) is (1) substituting at least one basic amino acid residue of the repeat domain of step (a) with a neutral or acidic amino acid residue; and
(2) substituting at least one neutral amino acid residue of the repeat domain of step (a) with an acidic amino acid;
   wherein said modified repeat domain has, among all amino acid residues comprised in the repeat domain, a percentage of basic amino acid residues equal to or lower than 12.0%.

In another more particular embodiment, said method according to the invention comprises steps (a) and (b),
wherein step (a) is providing an ankyrin repeat domain which has, among all amino acid residues comprised in the repeat domain, a percentage of basic amino acid residues higher than 12.0%; and
wherein step (b) is substituting at least one basic amino acid residue of the repeat domain of step (a) with a neutral or acidic amino acid residue;
   wherein said modified repeat domain has, among all amino acid residues comprised in the repeat domain, a percentage of basic amino acid residues equal to or lower than 12.0%.

In one embodiment, said method according to the invention comprises steps (a) and (b),
wherein step (a) is providing an ankyrin repeat domain which has, among all amino acid residues comprised in the repeat domain, a percentage of Arg and Lys residues higher than 8.0%; and
wherein step (b) comprises substituting at least one Arg or Lys residue of the repeat domain of step (a) with a neutral or acidic amino acid residue;
   wherein said modified repeat domain has, among all amino acid residues comprised in the repeat domain, a percentage of Arg and Lys residues equal to or lower than 8.0%.

In a more particular embodiment, said method according to the invention comprises steps (a) and (b),
wherein step (a) is providing an ankyrin repeat domain which has, among all amino acid residues comprised in the repeat domain, a percentage of Arg and Lys residues higher than 8.0%; and
wherein step (b) is (1) substituting at least one Arg or Lys residue of the repeat domain of step (a) with a neutral or acidic amino acid residue; and
(2) substituting at least one neutral amino acid residue of the repeat domain of step (a) with an acidic amino acid;
   wherein said modified repeat domain has, among all amino acid residues comprised in the repeat domain, a percentage of Arg and Lys residues equal to or lower than 8.0%.

In another more particular embodiment, said method according to the invention comprises steps (a) and (b),
wherein step (a) is providing an ankyrin repeat domain which has, among all amino acid residues comprised in the repeat domain, a percentage of Arg and Lys residues higher than 8.0%; and
wherein step (b) is substituting at least one Arg or Lys residue of the repeat domain of step (a) with a neutral or acidic amino acid residue;
   wherein said modified repeat domain has, among all amino acid residues comprised in the repeat domain, a percentage of Arg and Lys residues equal to or lower than 8.0%.

In one embodiment, said method according to the invention comprises steps (a) and (b),
wherein step (a) is providing an ankyrin repeat domain which has, among the framework residues comprised in the repeat domain, a percentage of basic amino acid residues higher than 9.6%; and
wherein step (b) comprises substituting at least one basic amino acid residue of the repeat domain of step (a) with a neutral or acidic amino acid residue;
   wherein said modified repeat domain has, among the framework residues comprised in the repeat domain, a percentage of basic amino acid residues equal to or lower than 9.6%.

In a more particular embodiment, said method according to the invention comprises steps (a) and (b),
wherein step (a) is providing an ankyrin repeat domain which has, among the framework residues comprised in the repeat domain, a percentage of basic amino acid residues higher than 9.6%; and
wherein step (b) is (1) substituting at least one basic amino acid residue among the framework residues of the repeat domain of step (a) with a neutral or acidic amino acid residue; and
(2) substituting at least one neutral amino acid residue of the repeat domain of step (a) with an acidic amino acid;
   wherein said modified repeat domain has, among the framework residues comprised in the repeat domain, a percentage of basic amino acid residues equal to or lower than 9.6%.

In another more particular embodiment, said method according to the invention comprises steps (a) and (b),
wherein step (a) is providing an ankyrin repeat domain which has, among the framework residues comprised in the repeat domain, a percentage of basic amino acid residues higher than 9.6%; and
wherein step (b) is substituting at least one basic amino acid residue among the framework residues of the repeat domain of step (a) with a neutral or acidic amino acid residue;
   wherein said modified repeat domain has, among the framework residues comprised in the repeat domain, a percentage of basic amino acid residues equal to or lower than 9.6%.

In one embodiment, said method according to the invention comprises steps (a) and (b),
wherein step (a) is providing an ankyrin repeat domain which has, among the framework residues comprised in the repeat domain, a percentage of Arg and Lys residues higher than 6.7%; and
wherein step (b) comprises substituting at least one Arg or Lys residue among the framework residues of the repeat domain of step (a) with a neutral or acidic amino acid residue;
   wherein said modified repeat domain has, among the framework residues comprised in the repeat domain, a percentage of Arg and Lys residues equal to or lower than 6.7%.

In a more particular embodiment, said method according to the invention comprises steps (a) and (b),
wherein step (a) is providing an ankyrin repeat domain which has, among the framework residues comprised in the repeat domain, a percentage of Arg and Lys residues higher than 6.7%; and
wherein step (b) is (1) substituting at least one Arg or Lys residue among the framework residues of the repeat domain of step (a) with a neutral or acidic amino acid residue; and
(2) substituting at least one neutral amino acid residue of the repeat domain of step (a) with an acidic amino acid;
   wherein said modified repeat domain has, among the framework residues comprised in the repeat domain, a percentage of Arg and Lys residues equal to or lower than 6.7%.

In another more particular embodiment, said method according to the invention comprises steps (a) and (b),
wherein step (a) is providing an ankyrin repeat domain which has, among the framework residues comprised in the repeat domain, a percentage of Arg and Lys residues higher than 6.7%; and
wherein step (b) is substituting at least one Arg or Lys residue among the framework residues of the repeat domain of step (a) with a neutral or acidic amino acid residue;
   wherein said modified repeat domain has, among the framework residues comprised in the repeat domain, a percentage of Arg and Lys residues equal to or lower than 6.7%.

In other embodiments of said method according to the invention, the repeat domain in step (a) has a KR/DE ratio higher than 0.44 and the modified repeat domain has a KR/DE ratio equal to or lower than 0.44.

In other embodiments of said method according to the invention, the repeat domain in step (a) has a KR/DE ratio among the framework residues higher than 0.36 and the modified repeat domain has a KR/DE ratio equal to or lower than 0.36.

In other embodiments of said method according to the invention, the repeat domain in step (a) has a KR/DE ratio among all residues of the N-terminal capping module higher than 0.66 and the modified repeat domain has a KR/DE ratio among all residues of the N-terminal capping module equal to or lower than 0.66.

In other embodiments of said method according to the invention, the repeat domain in step (a) has a KR/DE ratio higher than 0.44 and the modified repeat domain has a KR/DE ratio equal to or lower than 0.44, and/or the repeat domain in step (a) has a KR/DE ratio among the framework residues higher than 0.36 and the modified repeat domain has a KR/DE ratio equal to or lower than 0.36, and/or the repeat domain in step (a) has a KR/DE ratio among all residues of the N-terminal capping module higher than 0.66 and the modified repeat domain has a KR/DE ratio among all residues of the N-terminal capping module equal to or lower than 0.66.

In some embodiments, at least one, at least two, at least three, at least four, at least five, at least six, at least seven, at least eight, at least nine, at least ten, at least eleven, at least twelve, at least thirteen, at least fourteen or at least fifteen residues of the repeat domain in (a) are substituted.

In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, or at least 82% of the residues of the repeat domain in (a) are substituted. In cases where such percentages are to be converted into a corresponding number of residues, a conventional rounding of the computed residues to the closest integer may be used.

In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, or at least 82% of residues per repeat module in the repeat domain in (a) are substituted. In said embodiments, the per repeat module wording includes N-terminal capping module and/or C-terminal capping module and/or internal repeat module(s).

In one embodiment, the method according to the invention further comprises a step of linking a drug moiety to the modified repeat domain. In some embodiments, the drug moiety is linked covalently or non-covalently to the modified repeat domain. In a more particular embodiment, the drug moiety is linked to the modified repeat domain by a chelator. Appropriate chelators may be selected depending on the application. In a particular embodiment, said chelator is DTPA. In a more particular embodiment, said drug moiety is a radionuclide. The choice of said radionuclide may depend on the intended application of said modified repeat domain (e.g. diagnostic vs. therapeutic).

In on embodiment, said drug moiety is a therapeutic moiety. In one embodiment, said therapeutic moiety is a toxin. In one embodiment, said therapeutic moiety is a radionuclide. In a more particular embodiment, said radionuclide is indium-111.

In other embodiments, said drug moiety is a diagnostic moiety. In a more particular embodiment, said diagnostic moiety is a fluorophore, a chromophore, an imaging agent or a radionuclide.

Residues selected for substitutions can be located at randomized or non-randomized positions of the repeat domain. Accordingly, in some embodiments, the substituted residues are selected among residues located at non-randomized positions of said repeat domain. In other embodiments the substituted residues are selected among residues located at randomized positions of said repeat domain. In some embodiments the substituted residues are selected among residues located at randomized and non-randomized positions of said repeat domain. In other embodiments, residues selected for substitutions can be framework residues or target interaction residues. Accordingly, in some embodiments, the substituted residues are selected among the framework residues comprised in said repeat domain. In other embodiments the substituted residues are selected among the target interaction residues comprised in said repeat domain. In other embodiments the substituted residues are selected among all residues comprised in said repeat domain. All residues in this sense shall mean any of the target interaction residues or framework residues comprised in said repeat domain. In some embodiments, the randomized positions of the repeat domain correspond to the positions of potential target interaction residues. In further embodiments, the non-randomized positions correspond to the positions of framework residues. Preferred positions of framework residues are shown in Table 4. Table 5 shows preferred positions of potential target interaction residues.

TABLE 5

| | Representative | Position of potential target |
|---|---|---|
| Repeat modules | sequence | interaction residues |
| N-terminal capping | SEQ ID NO: 43 | 4, 8, 11 and 12 |
| module | | |
| Internal repeat | SEQ ID NO: 48 | 3, 4, 6, 11, 14 and 15 |
| module | | |
| C-terminal capping | SEQ ID NO: 54 | 3, 4, 6, 14 and 15 |
| module | | |

In certain embodiments, said substitution is made outside the structural core residues of the ankyrin repeat domain, e.g. in the beta loops that connect the alpha-helices. In certain embodiments, said substitution is made within the structural core residues of the ankyrin repeat domain. For example, the ankyrin repeat domain of step (a) may comprise a sequence disclosed herein such as for instance a consensus sequence selected from: XDXXGXTPLXXAXXXGXLXIXXVLLXAGADVNA (SEQ ID NO: 32), DLGXXLLXAAXXGQLDXVRXLLXAGADVNA (SEQ ID NO: 20) and QDXXGXTPAXXAAXXGXXXIAXVLQXAA (SEQ ID NO: 41), wherein “X” denotes any amino acid. In one embodiment, the substitution is made to residues designated as “X”. In another embodiment, said substitution is made outside the residues designated as “X”. In preferred embodiments, the substitute amino acid is not cysteine, glycine, or proline. In some embodiments, the term “any amino acid” as defined for certain positions “X” in the disclosed sequences of the invention corresponds to “any naturally occurring amino acid”.

The repeat domain of step (a) may be any repeat domain commonly known to a skilled person in the art. Preferably, said repeat domain of step (a) is a designed ankyrin repeat domain. In some embodiments, said repeat domain of step (a) comprises an N-terminal capping module, one or more internal repeat module(s) and a C-terminal capping module. Preferably, said repeat domain of step (a) comprises 1, 2, 3, 4, 5, 6, 7, 8 or 9 internal repeat modules, most preferably said repeat domain comprises 2, 3 or 4 internal repeat modules. In some embodiments, the repeat domain of step (a) may comprise a sequence selected from SEQ ID NOs: 19 to 25, 57 to 60, 65 to 81, 31 to 35, 61 to 62, 92 to 94, 40 to 42, 63 to 64 and 82 to 91. In some embodiments, each internal repeat module comprised in the repeat domain of step (a) may independently comprise a sequence selected from SEQ ID NOs: 31 to 35, 61 to 62 and 92 to 94. Alternatively or in addition, the N-terminal capping module comprised in the repeat domain of step (a) may comprise a sequence selected from SEQ ID NOs: 19 to 25, 57 to 60, 65 to 81. Alternatively or in addition, the C-terminal capping module comprised in the repeat domain of step (a) may comprise a sequence selected from SEQ ID NOs: 40 to 42, 63 to 64 and 82 to 91.

In some embodiment, the modified repeat domain generated by the method of the invention comprises one internal repeat module, two internal repeat modules, three internal repeat modules, or four internal repeat modules.

In one embodiment said repeat domain of step (a) binds specifically to a target. In some embodiments, said repeat domain of step (a) binds to said target with a dissociation constant (K_{D}) of about 10⁻⁵ M or less, about 10⁻⁶ M or less, about 10⁻⁷ M or less, about 10⁻⁷ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less.

In a preferred embodiment, the modified repeat domain generated by the method of the invention maintains the binding characteristics of the repeat domain of step (a). Accordingly, in some embodiments said modified repeat domain binds said target with a K_{D} that is (1) about equal to the K_{D} with which said repeat domain of step (a) binds said target or (2) less than 100 fold, less than 10 fold, less than 5 fold or less than 2 fold higher than the K_{D} with which said repeat domain of step (a) binds said target.

In some embodiments, said modified repeat domain generated by the method of the invention has an isoelectric point (pI) at least 0.1 pH points lower than the repeat domain of step (a). In some embodiments, said pI is at least 0.1, at least 0.2, at least 0.3, at least 0.4, at least 0.5, at least 0.6, at least 0.7, at least 0.8, at least 0.9, at least 1.0 or at least 1.1 pH points lower that the repeat domain of step (a).

In one embodiment, the modified repeat domain generated by the method of the invention is for use in a drug, wherein said drug comprises said modified repeat domain linked to a drug moiety, and wherein said drug moiety optionally is a radionuclide or a cytotoxic agent.

Properties described in relation to the designed ankyrin repeat domain of the invention may similarly apply to the modified repeat domain obtained by the method of the invention.

In some embodiments, the modified repeat domain generated by the method of the invention has a pI equal to or lower than pH 4.9, equal to or lower than pH 4.8, equal to or lower than pH 4.7, equal to or lower than pH 4.6, equal to or lower than pH 4.5, equal to or lower than pH 4.4, equal to or lower than pH 4.3, equal to or lower than pH 4.2, equal to or lower than pH 4.1, equal to or lower than pH 4.0, equal to or lower than pH 3.9, equal to or lower than pH 3.8, equal to or lower than pH 3.7, equal to or lower than pH 3.6, or equal to or lower than pH 3.5.

In some embodiments, the modified repeat domain generated by the method of the invention has a pI equal to or lower than pH 4.53, equal to or lower than pH 4.52, equal to or lower than pH 4.50, equal to or lower than pH 4.48, equal to or lower than pH 4.46, equal to or lower than pH 4.44, equal to or lower than pH 4.42, equal to or lower than pH 4.40, equal to or lower than pH 4.38, equal to or lower than pH 4.36, equal to or lower than pH 4.34, equal to or lower than pH 4.32, equal to or lower than pH 4.30, equal to or lower than pH 4.28, equal to or lower than pH 4.26, equal to or lower than pH 4.24, equal to or lower than pH 4.22, equal to or lower than pH 4.20, equal to or lower than pH 4.18, equal to or lower than pH 4.16, equal to or lower than pH 4.14, equal to or lower than pH 4.12, equal to or lower than pH 4.10, equal to or lower than pH 4.08, equal to or lower than pH 4.06, equal to or lower than pH 4.04, equal to or lower than pH 4.02, equal to or lower than pH 4.00, equal to or lower than pH 3.98, equal to or lower than pH 3.96, equal to or lower than pH 3.94, equal to or lower than pH 3.92, equal to or lower than pH 3.90, equal to or lower than pH 3.88, equal to or lower than pH 3.86, equal to or lower than pH 3.84, equal to or lower than pH 3.82, equal to or lower than pH 3.80, equal to or lower than pH 3.78, equal to or lower than pH 3.76, equal to or lower than pH 3.74, equal to or lower than pH 3.72, equal to or lower than pH 3.70, equal to or lower than pH 3.68, equal to or lower than pH 3.66, equal to or lower than pH 3.64, equal to or lower than pH 3.62, equal to or lower than pH 3.60, equal to or lower than pH 3.58, equal to or lower than pH 3.56, equal to or lower than pH 3.54, equal to or lower than pH 3.52, or equal to or lower than pH 3.50.

In some embodiments, the modified repeat domain generated by the method of the invention has a pI in a range between pH 3.00 and pH 4.60, between pH 3.10 and pH 4.60, between pH 3.20 and pH 4.60, between pH 3.30 and pH 4.60, between pH 3.40 and pH 4.60, between pH 3.50 and pH 4.60, between pH 3.50 and pH 4.58, between pH 3.50 and pH 4.56, between pH 3.50 and pH 4.54, between pH 3.50 and pH 4.53, between pH 3.50 and pH 4.52, between pH 3.50 and pH 4.50, between pH 3.50 and pH 4.48, between pH 3.50 and pH 4.46, between pH 3.50 and pH 4.44, between pH 3.50 and pH 4.42, between pH 3.50 and pH 4.40, between pH 3.50 and pH 4.38, between pH 3.50 and pH 4.36, between pH 3.50 and pH 4.34, between pH 3.50 and pH 4.32, between pH 3.50 and pH 4.30, between pH 3.60 and pH 4.60, between pH 3.60 and pH 4.58, between pH 3.60 and pH 4.56, between pH 3.60 and pH 4.54, between pH 3.60 and pH 4.53, between pH 3.60 and pH 4.52, between pH 3.60 and pH 4.50, between pH 3.60 and pH 4.48, between pH 3.60 and pH 4.46, between pH 3.60 and pH 4.44, between pH 3.60 and pH 4.42, between pH 3.60 and pH 4.40, between pH 3.60 and pH 4.38, between pH 3.60 and pH 4.36, between pH 3.60 and pH 4.34, between pH 3.60 and pH 4.32, between pH 3.60 and pH 4.30, between pH 3.70 and pH 4.60, between pH 3.70 and pH 4.58, between pH 3.70 and pH 4.56, between pH 3.70 and pH 4.54, between pH 3.70 and pH 4.53, between pH 3.70 and pH 4.52, between pH 3.70 and pH 4.50, between pH 3.70 and pH 4.48, between pH 3.70 and pH 4.46, between pH 3.70 and pH 4.44, between pH 3.70 and pH 4.42, between pH 3.70 and pH 4.40, between pH 3.70 and pH 4.38, between pH 3.70 and pH 4.36, between pH 3.70 and pH 4.34, between pH 3.70 and pH 4.32, or between pH 3.70 and pH 4.30.

In some embodiments, the modified repeat domain generated by the method of the invention has a percentage of basic amino acid residues equal to or lower than 12.0%, equal to or lower than 11.5%, equal to or lower than 11.0%, equal to or lower than 10.5%, equal to or lower than 10.0%, equal to or lower than 9.5%, or equal to or lower than 9.0%.

In some embodiments, the modified repeat domain generated by the method of the invention has a percentage of basic amino acid residues equal to or lower than 8.87%, equal to or lower than 8.80%, equal to or lower than 8.70%, equal to or lower than 8.60%, equal to or lower than 8.50%, equal to or lower than 8.40%, equal to or lower than 8.30%, equal to or lower than 8.20%, equal to or lower than 8.10%, equal to or lower than 8.00%, equal to or lower than 7.90%, equal to or lower than 7.80%, equal to or lower than 7.70%, equal to or lower than 7.60%, equal to or lower than 7.50%, equal to or lower than 7.40%, equal to or lower than 7.30%, equal to or lower than 7.20%, equal to or lower than 7.10%, equal to or lower than 7.00%, equal to or lower than 6.90%, equal to or lower than 6.80%, equal to or lower than 6.70%, equal to or lower than 6.60%, equal to or lower than 6.50%, equal to or lower than 6.40%, equal to or lower than 6.30%, equal to or lower than 6.20%, equal to or lower than 6.10%, equal to or lower than 6.00%, equal to or lower than 5.90%, equal to or lower than 5.80%, equal to or lower than 5.70%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, or equal to or lower than 4.80%.

In some embodiments, the modified repeat domain generated by the method of the invention has a percentage of Arg and Lys residues equal to or lower than 8.0%, equal to or lower than 7.5%, equal to or lower than 7.0%, equal to or lower than 6.5%, or equal to or lower than 6.0%.

In some embodiments, the modified repeat domain generated by the method of the invention has a percentage of Arg and Lys residues equal to or lower than 5.65%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, equal to or lower than 4.80%, equal to or lower than 4.70%, equal to or lower than 4.60%, equal to or lower than 4.50%, equal to or lower than 4.40%, equal to or lower than 4.30%, equal to or lower than 4.20%, equal to or lower than 4.10%, equal to or lower than 4.00%, equal to or lower than 3.90%, equal to or lower than 3.80%, equal to or lower than 3.70%, equal to or lower than 3.60%, equal to or lower than 3.50%, equal to or lower than 3.40%, equal to or lower than 3.30%, equal to or lower than 3.20%, equal to or lower than 3.10%, equal to or lower than 3.00%, equal to or lower than 2.90%, equal to or lower than 2.80%, equal to or lower than 2.70%, equal to or lower than 2.60%, equal to or lower than 2.50%, equal to or lower than 2.40%, equal to or lower than 2.30%, equal to or lower than 2.20%, equal to or lower than 2.10%, equal to or lower than 2.00%, equal to or lower than 1.90%, equal to or lower than 1.80%, equal to or lower than 1.70%, equal to or lower than 1.60%, equal to or lower than 1.50%, equal to or lower than 1.40%, equal to or lower than 1.30%, equal to or lower than 1.20%, equal to or lower than 1.10%, equal to or lower than 1.00%, equal to or lower than 0.90%, equal to or lower than 0.80%, equal to or lower than 0.70%, equal to or lower than 0.60%, equal to or lower than 0.50%, equal to or lower than 0.40%, equal to or lower than 0.30%, equal to or lower than 0.20%, or equal to or lower than 0.10%.

In some embodiments, the modified repeat domain generated by the method of the invention has a percentage of basic amino acid residues among the framework residues equal to or lower than 9.6%, equal to or lower than 9.4%, equal to or lower than 9.2%, equal to or lower than 9.0%, equal to or lower than 8.8%, equal to or lower than 8.6%, equal to or lower than 8.4%, equal to or lower than 8.2%, or equal to or lower than 8.0%, equal to or lower than 7.8%.

In some embodiments, the modified repeat domain generated by the method of the invention has a percentage of basic amino acid residues among the framework residues equal to or lower than 7.77%, equal to or lower than 7.70%, equal to or lower than 7.60%, equal to or lower than 7.50%, equal to or lower than 7.40%, equal to or lower than 7.30%, equal to or lower than 7.20%, equal to or lower than 7.10%, equal to or lower than 7.00%, equal to or lower than 6.90%, equal to or lower than 6.80%, equal to or lower than 6.70%, equal to or lower than 6.60%, equal to or lower than 6.50%, equal to or lower than 6.40%, equal to or lower than 6.30%, equal to or lower than 6.20%, equal to or lower than 6.10%, equal to or lower than 6.00%, equal to or lower than 5.90%, equal to or lower than 5.80%, equal to or lower than 5.70%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, equal to or lower than 4.80%, equal to or lower than 4.70%, equal to or lower than 4.60%, or equal to or lower than 4.50%.

In some embodiments, the designed ankyrin repeat domain of the invention has a percentage of Arg and Lys residues among the framework residues equal to or lower than 6.7%, equal to or lower than 6.6%, equal to or lower than 6.5%, equal to or lower than 6.4%, equal to or lower than 6.3%, equal to or lower than 6.2%, equal to or lower than 6.1%, equal to or lower than 6.0%, or equal to or lower than 5.9%.

In some embodiments, the modified repeat domain generated by the method of the invention has a percentage of Arg and Lys residues among the framework residues equal to or lower than 5.83%, equal to or lower than 5.80%, equal to or lower than 5.70%, equal to or lower than 5.60%, equal to or lower than 5.50%, equal to or lower than 5.40%, equal to or lower than 5.30%, equal to or lower than 5.20%, equal to or lower than 5.10%, equal to or lower than 5.00%, equal to or lower than 4.90%, equal to or lower than 4.80%, equal to or lower than 4.70%, equal to or lower than 4.60%, equal to or lower than 4.50%, equal to or lower than 4.40%, equal to or lower than 4.30%, equal to or lower than 4.20%, equal to or lower than 4.10%, equal to or lower than 4.00%, equal to or lower than 3.90%, equal to or lower than 3.80%, equal to or lower than 3.70%, equal to or lower than 3.60%, equal to or lower than 3.50%, equal to or lower than 3.40%, equal to or lower than 3.30%, equal to or lower than 3.20%, equal to or lower than 3.10%, equal to or lower than 3.00%, equal to or lower than 2.90%, equal to or lower than 2.80%, equal to or lower than 2.70%, equal to or lower than 2.60%, equal to or lower than 2.50%, equal to or lower than 2.40%, equal to or lower than 2.30%, equal to or lower than 2.20%, equal to or lower than 2.10%, equal to or lower than 2.00%, equal to or lower than 1.90%, equal to or lower than 1.80%, equal to or lower than 1.70%, equal to or lower than 1.60%, equal to or lower than 1.50%, equal to or lower than 1.40%, equal to or lower than 1.30%, equal to or lower than 1.20%, equal to or lower than 1.10%, equal to or lower than 1.00%, equal to or lower than 0.90%, equal to or lower than 0.80%, equal to or lower than 0.70%, equal to or lower than 0.60%, equal to or lower than 0.50%, equal to or lower than 0.40%, equal to or lower than 0.30%, equal to or lower than 0.20%, or equal to or lower than 0.10%.

Modified repeat domain generated by the method of the invention may comprise an N-terminal capping module, a C-terminal capping module and at least one internal repeat module. Preferably, said modified repeat domains comprise 1, 2, 3, 4, 5, 6, 7, 8 or 9 internal repeat module(s). More preferably, said modified repeat domains of the invention comprise 1, 2, 3 or 4 internal repeat modules.

In some embodiments, the total number of basic amino acid residues comprised in the modified repeat domain generated by the method of the invention can be expressed as a function based on the number of repeat modules comprised in said repeat domain. In one embodiment, said modified repeat domain has a total number of basic amino acid residues equal to or lower than n, wherein n=R, n=1+R, n=2+R, n=3+R, n=4+R, n=5+R, n=6+R, n=7+R, n=8+R, n=9+R, n=2R, n=1+2R, n=2+2R, n=3+2R, n=4+2R, n=5+2R, n=6+2R, n=7+2R, n=8+2R, n=9+2R, n=3R, n=1+3R, n=2+3R, n=3+3R, n=4+3R, n=5+3R, n=6+3R, n=7+3R, n=8+3R, n=9+3R, n=4R, n=1+4R, n=2+4R, n=3+4R, n=4+4R, n=5+4R, n=6+4R, n=7+4R, n=8+4R, n=9+4R, n=5R, n=1+5R, n=2+5R, n=3+5R, n=4+5R, n=5+5R, n=6+5R, n=7+5R, n=8+5R, n=9+5R, n=6R, n=1+6R, n=2+6R, n=3+6R, n=4+6R, n=5+6R, n=6+6R, n=7+6R, n=8+6R, or n=9+6R, wherein R is the number of internal repeat modules comprised in said modified repeat domain. In such embodiments, “xR” means x multiplied by R.

In another embodiment, the total number of Arg and Lys residues comprised in the modified repeat domain generated by the method of the invention is equal to or lower than m, wherein m=R, m=1+R, m=2+R, m=3+R, m=4+R, m=5+R, m=6+R, m=7+R, m=8+R, m=9+R, m=2R, m=1+2R, m=2+2R, m=3+2R, m=4+2R, m=5+2R, m=6+2R, m=7+2R, m=8+2R, m=9+2R, m=3R, m=1+3R, m=2+3R, m=3+3R, m=4+3R, m=5+3R, m=6+3R, m=7+3R, m=8+3R, m=9+3R, m=4R, m=1+4R, m=2+4R, m=3+4R, m=4+4R, m=5+4R, m=6+4R, m=7+4R, m=8+4R, or m=9+4R, wherein R is the number of internal repeat modules comprised in said modified repeat domain.

In some embodiments, the modified repeat domain generated by the method of the invention has a total number of basic amino acid residues among the framework residues equal to or lower than x, wherein x=R, x=1+R, x=2+R, x=3+R, x=4+R, x=5+R, x=6+R, x=7+R, x=2R, x=1+2R, x=2+2R, x=3+2R, x=4+2R, x=5+2R, x=6+2R, x=7+2R, x=3R, x=1+3R, x=2+3R, x=3+3R, x=4+3R, x=5+3R, x=6+3R, or x=7+3R, wherein R is the number of internal repeat modules comprised in said modified repeat domain.

In other embodiments, the modified repeat domain generated by the method of the invention has a total number of Arg and Lys residues among the framework residues equal to or lower than y, wherein y=1+R, y=2+R, y=3+R, y=4+R, y=5+R, y=6+R, y=2R, y=1+2R, y=2+2R, y=3+2R, y=4+2R, y=5+2R, or y=6+2R, wherein R is the number of internal repeat modules comprised in said modified repeat domain.

Preferably, modified repeat domain generated by the method of the invention bind specifically to a target.

In preferred embodiments, said modified repeat domain binds to a target with a dissociation constant (K_{D}) of about 10⁻⁵ M or less, about 10⁻⁶ M or less, about 10 ⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less.

In some embodiments, the modified repeat domain generated by the method of the invention has a KR/DE ratio equal to or lower than 0.44, equal to or lower than 0.42, equal to or lower than 0.40, equal to or lower than 0.38, equal to or lower than 0.36, equal to or lower than 0.34, equal to or lower than 0.32, equal to or lower than 0.30, equal to or lower than 0.28, equal to or lower than 0.26, equal to or lower than 0.24, equal to or lower than 0.22, equal to or lower than 0.20, equal to or lower than 0.18, equal to or lower than 0.16, equal to or lower than 0.14, equal to or lower than 0.12, equal to or lower than 0.10, or equal to or lower than 0.08. Preferably, said KR/DE ratio is equal to or lower than 0.3.

In some embodiments, the modified repeat domain generated by the method of the invention has a KR/DE ratio among the framework residues equal to or lower than 0.36, equal to or lower than 0.34, equal to or lower than 0.32, equal to or lower than 0.30, equal to or lower than 0.28, equal to or lower than 0.26, equal to or lower than 0.24, equal to or lower than 0.22, equal to or lower than 0.20, equal to or lower than 0.18, equal to or lower than 0.16, equal to or lower than 0.14, equal to or lower than 0.12, equal to or lower than 0.10, or equal to or lower than 0.08. Preferably, said KR/DE ratio among the framework residues is equal to or lower than 0.25.

In some embodiments, the modified repeat domain generated by the method of the invention has a KR/DE ratio among all residues of the N-terminal capping module equal to or lower than 0.66, equal to or lower than 0.64, equal to or lower than 0.62, equal to or lower than 0.60, equal to or lower than 0.58, equal to or lower than 0.56, equal to or lower than 0.54, equal to or lower than 0.52, equal to or lower than 0.50, equal to or lower than 0.48, equal to or lower than 0.46, equal to or lower than 0.44, equal to or lower than 0.42, equal to or lower than 0.40, equal to or lower than 0.38, equal to or lower than 0.36, equal to or lower than 0.34, equal to or lower than 0.32, equal to or lower than 0.30, equal to or lower than 0.28, equal to or lower than 0.26, equal to or lower than 0.24, equal to or lower than 0.22, equal to or lower than 0.20, equal to or lower than 0.18, equal to or lower than 0.16, equal to or lower than 0.14, equal to or lower than 0.12, equal to or lower than 0.10, equal to or lower than 0.08, equal to or lower than 0.06, equal to or lower than 0.04, or equal to or lower than 0.02. Preferably said KR/DE ratio among all residues of the N-terminal capping module is equal to or lower than 0.5.

In some embodiments, the modified repeat domain generated by the method of the invention has no Arg residue and no Lys residue within the framework residue positions.

In some embodiments, the modified repeat domain generated by the method of the invention comprises two internal repeat modules. In some embodiments, said modified repeat domain comprising two internal repeat modules may have a total number of basic amino acid residues equal to or lower than 1, 2, 3, 4, 5, 6, 7, 8, 9, 10, 11, 12, 13 or 14. Preferably said total number of basic residues is equal to or lower than 14.

More preferably said total number of basic residues is equal to or lower than 10. In another particular embodiment, said modified repeat domain comprising two internal repeat modules may have a total number of Arg and Lys residues equal to or lower than 1, 2, 3, 4, 5, 6, 7, 8 or 9. Preferably said total number of Arg and Lys residues is equal to or lower than 9. More preferably, said total number of Arg and Lys residues is equal to or lower than 5. In another particular embodiment, said modified repeat domain comprising two internal repeat modules may have a total number of Arg and Lys residues among the framework residues equal to or lower than 1, 2, 3, 4, 5 or 6. Preferably said total number of Arg and Lys residues among the framework residues is equal to or lower than 6. More preferably, said total number of Arg and Lys residues among the framework residues is equal to or lower than 5.

In some embodiments, the modified repeat domain generated by the method of the invention comprises an N-terminal capping module having a sequence selected from the group consisting of (1) SEQ ID NOs: 25 and 115 to 122 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 25 and 115 to 122 are substituted by another amino acid.

In some embodiments, said N-terminal capping module comprises a sequence selected from the group consisting of (1) SEQ ID NOs: 115 and 121 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 115 and 121 are substituted by another amino acid.

In further embodiments, said N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid.

Alternatively or in addition to the above defined N-terminal capping modules, in some embodiments, the modified repeat domain generated by the method of the invention comprises a C-terminal capping module having a sequence selected from the group consisting of (1) SEQ ID NOs: 123 to 130 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 to 130 are substituted by another amino acid.

In some embodiments, said C-terminal capping module comprises a sequence selected from the group consisting of (1) SEQ ID NOs: 123 and 126 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 and 126 are substituted by another amino acid.

In further embodiments, said C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In a particular embodiment, the modified repeat domain generated by the method of the invention comprises:

an N-terminal capping module having a sequence selected from the group consisting of (1) SEQ ID NOs: 115 and 121 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 115 and 121 are substituted by another amino acid, and a C-terminal capping module having a sequence selected from the group consisting of (1) SEQ ID NOs: 123 and 126 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 123 and 126 are substituted by another amino acid.

In another particular embodiment, the modified repeat domain generated by the method of the invention comprises:
an N-terminal capping module having a sequence selected from the group consisting of (1) SEQ ID NO: 115 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 115 are substituted by another amino acid, and a C-terminal capping module having a sequence selected from the group consisting of (1) SEQ ID NO: 123 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in SEQ ID NO: 123 are substituted by another amino acid.

In further embodiments, the modified repeat domain generated by the method of the invention comprises one or more internal repeat module independently comprises a sequence selected from the group consisting of (1) SEQ ID NO: 31 and (2) sequences in which up to 5, 4, 3, 2 or 1 framework residues other than positions 1, 10, 13, 17, 19, 21, 22 and 26 in SEQ ID NO: 31 are substituted by another amino acid, and/or said N-terminal capping module comprises a sequence selected from the group consisting of (1) SEQ ID NO: 19 and (2) sequences in which up to 5, 4, 3, 2 or 1 framework residues other than positions 5, 17, 20 and 23 in SEQ ID NOs: 19 are substituted by another amino acid, and/or said C-terminal capping module comprises a sequence selected from the group consisting of (1) SEQ ID NO: 40 and (2) sequences in which up to 5, 4, 3, 2 or 1 framework residues other than positions 10, 11, 17, 18, 19, 22 and 26 in SEQ ID NO: 40 are substituted by another amino acid.

In particular embodiments, the modified repeat domain of the invention comprises a sequence selected from SEQ ID NOs: 19 to 25, 57 to 60, 65 to 81, 31 to 35, 61 to 62, 92 to 94, 40 to 42, 63 to 64 and 82 to 91.

In particular embodiments, each internal repeat module comprised in the modified repeat domain generated by the method of the invention may independently comprise a sequence selected from SEQ ID NOs: 31 to 35, 61 to 62 and 92 to 94. Alternatively or in addition, the N-terminal capping module comprised in the modified repeat domain generated by the method of the invention may comprise a sequence selected from SEQ ID NOs: 19 to 25, 57 to 60 and 65 to 81. Alternatively or in addition, the C-terminal capping module comprised in the modified repeat domain generated by the method of the invention may comprise a sequence selected from SEQ ID NOs: 40 to 42, 63 to 64 and 82 to 91.

In particular embodiments, each internal repeat module comprised in the modified repeat domain generated by the method of the invention independently comprises a sequence selected from SEQ ID NOs: 31 to 35, 61 to 62, 92 to 94 and 131 to 142. Alternatively or in addition, the N-terminal capping module comprised in the modified repeat domain generated by the method of the invention comprises a sequence selected from SEQ ID NOs: 19 to 25, 57 to 60, 65 to 81 and 115 to 122. Alternatively or in addition, the C-terminal capping module comprised in the modified repeat domain generated by the method of the invention comprises a sequence selected from SEQ ID NOs: 40 to 42, 63 to 64, 82 to 91 and 123 to 130.

In further embodiments, each internal repeat module comprised in the modified repeat domain generated by the method of the invention independently comprises a sequence selected from the group consisting of (1) SEQ ID NOs: 31 to 35, 61 to 62 and 92 to 94 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 31 to 35, 61 to 62 and 92 to 94 are substituted by another amino acid. Alternatively or in addition, the N-terminal capping module comprised in the modified repeat domain generated by the method of the invention comprises a sequence selected from the group consisting of (1) SEQ ID NOs: 19 to 25, 57 to 60 and 65 to 81 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 19 to 25, 57 to 60 and 65 to 81 are substituted by another amino acid. Alternatively or in addition, the C-terminal capping module comprised in the modified repeat domain generated by the method of the invention comprises a sequence selected from the group consisting of (1) SEQ ID NOs: 40 to 42, 63 to 64 and 82 to 91 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 40 to 42, 63 to 64 and 82 to 91 are substituted by another amino acid.

In particular embodiments, each internal repeat module comprised in the modified repeat domain generated by the method of the invention independently comprises a sequence selected from the group consisting of (1) SEQ ID NOs: 31 to 32 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 31 to 32 are substituted by another amino acid. Alternatively or in addition, the N-terminal capping module comprised in the modified repeat domain generated by the method of the invention comprises a sequence selected from the group consisting of (1) SEQ ID NOs: 19 to 20 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 19 to 20 are substituted by another amino acid. Alternatively or in addition, the C-terminal capping module comprised in the modified repeat domain generated by the method of the invention comprises a sequence selected from the group consisting of (1) SEQ ID NOs: 40 to 41 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any of SEQ ID NOs: 40 to 41 are substituted by another amino acid.

In further embodiments, each internal repeat module comprised in the modified repeat domain generated by the method of the invention may independently comprises a sequence selected from the group consisting of (1) SEQ ID NOs: 26 to 30 and (2) sequences in which 1, 2, 3, 4, 5, 6, 7, 8 or 9 amino acids in any of SEQ ID NOs: 26 to 30 are substituted by another amino acid. Alternatively or in addition, the N-terminal capping module comprised in the modified repeat domain generated by the method of the invention may comprise a sequence selected from the group consisting of (1) SEQ ID NOs: 15 to 18 and (2) sequences in which 1, 2, 3, 4, 5, 6, 7, 8 or 9 amino acids in any of SEQ ID NOs: 15 to 18 are substituted by another amino acid. Alternatively or in addition, the C-terminal capping module comprised in the modified repeat domain generated by the method of the invention may comprise a sequence selected from the group consisting of (1) SEQ ID NOs: 36 to 39 and (2) sequences in which 1, 2, 3, 4, 5, 6, 7, 8 or 9 amino acids in any of SEQ ID NOs: 36 to 39 are substituted by another amino acid.

In further embodiments, each internal repeat module comprised in the modified repeat domain generated by the method of the invention may also independently comprise a sequence selected from the group consisting of (1) SEQ ID NOs: 46 to 52 and (2) sequences in which 1, 2, 3, 4, 5, 6, 7, 8 or 9 amino acids in any of SEQ ID NOs: 46 to 52 are substituted by another amino acid. Alternatively or in addition, the N-terminal capping module comprised in the modified repeat domain generated by the method of the invention may also comprise a sequence selected from the group consisting of (1) SEQ ID NOs: 43 to 45 and (2) sequences in which 1, 2, 3, 4, 5, 6, 7, 8 or 9 amino acids in any of SEQ ID NOs: 43 to 45 are substituted by another amino acid. Alternatively or in addition, the C-terminal capping module comprised in the modified repeat domain generated by the method of the invention may also comprise a sequence selected from the group consisting of (1) SEQ ID NOs: 53 to 56 and (2) sequences in which 1, 2, 3, 4, 5, 6, 7, 8 or 9 amino acids in any of SEQ ID NOs: 53 to 56 are substituted by another amino acid.

In further embodiments, each internal repeat module comprised in the modified repeat domain generated by the method of the invention independently comprises a sequence selected from the group consisting of (1) SEQ ID NOs: 26 to 30 and 46 to 52, and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 26 to 30 and 46 to 52 are substituted by another amino acid. Alternatively or in addition, the N-terminal capping module comprised in the modified repeat domain generated by the method of the invention comprises a sequence selected from the group consisting of (1) SEQ ID NOs: 15 to 18, 43 to 45 and 99 to 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 15 to 18, 43 to 45 and 99 to 101 are substituted by another amino acid. Alternatively or in addition, the C-terminal capping module comprised in the modified repeat domain generated by the method of the invention comprises a sequence selected from the group consisting of (1) SEQ ID NOs: 36 to 39, 53 to 56 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 36 to 39, 53 to 56 and 102 to 103 are substituted by another amino acid.

In further embodiments, each internal repeat module comprised in the modified repeat domain generated by the method of the invention independently comprises a sequence selected from the group consisting of (1) SEQ ID NOs: 26 to 30 and 47 to 48, and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 26 to 30 and 47 to 48 are substituted by another amino acid. Alternatively or in addition, the N-terminal capping module comprised in the modified repeat domain generated by the method of the invention comprises a sequence selected from the group consisting of (1) SEQ ID NOs: 15 to 18, 43 and 99 to 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 15 to 18, 43 and 99 to 101 are substituted by another amino acid. Alternatively or in addition, the C-terminal capping module comprised in the modified repeat domain generated by the method of the invention comprises a sequence selected from the group consisting of (1) SEQ ID NOs: 36 to 39, 54 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ

ID NOs: 36 to 39, 54 and 102 to 103 are substituted by another amino acid.

In further embodiments, each internal repeat module comprised in the modified repeat domain generated by the method of the invention independently comprises a sequence selected from the group consisting of (1) SEQ ID NOs: 47 to 48, and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 47 to 48 are substituted by another amino acid. Alternatively or in addition, the N-terminal capping module comprised in the modified repeat domain generated by the method of the invention comprises a sequence selected from the group consisting of (1) SEQ ID NOs: 43, 100, 99 and 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 43, 100, 99 and 101 are substituted by another amino acid. Alternatively or in addition, the C-terminal capping module comprised in the modified repeat domain generated by the method of the invention comprises a sequence selected from the group consisting of (1) SEQ ID NOs: 54 and 102 to 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 54 and 102 to 103 are substituted by another amino acid.

In further embodiments, each internal repeat module comprised in the modified repeat domain generated by the method of the invention independently comprises a sequence selected from the group consisting of (1) SEQ ID NOs: 29, 30 and 47 to 50 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 29, 30 and 47 to 50 are substituted by another amino acid. Alternatively or in addition, the N-terminal capping module comprised in the modified repeat domain generated by the method of the invention comprises a sequence selected from the group consisting of (1) SEQ ID NOs: 18, 43, 44, and 99 to 101 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 18, 43, 44, and 99 to 101 are substituted by another amino acid. Alternatively or in addition, the C-terminal capping module comprised in the modified repeat domain generated by the method of the invention comprises a sequence selected from the group consisting of (1) SEQ ID NOs: 39, 54, 55, 102 and 103 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 39, 54, 55, 102 and 103 are substituted by another amino acid.

In further embodiments, each internal repeat module comprised in the modified repeat domain generated by the method of the invention independently comprises a sequence selected from the group consisting of (1) SEQ ID NOs: 26 to 28 and 46 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 26 to 28 and 46 are substituted by another amino acid. Alternatively or in addition, the N-terminal capping module comprised in the modified repeat domain generated by the method of the invention comprises a sequence selected from the group consisting of (1) SEQ ID NOs: 15 to 17 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 15 to 17 are substituted by another amino acid.

Alternatively or in addition, the C-terminal capping module comprised in the modified repeat domain generated by the method of the invention comprises a sequence selected from the group consisting of (1) SEQ ID NOs: 36 to 38 and 53 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 36 to 38 and 53 are substituted by another amino acid.

In further embodiments, each internal repeat module comprised in the modified repeat domain generated by the method of the invention independently comprises a sequence selected from the group consisting of (1) SEQ ID NOs: 51 to 52 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in any of SEQ ID NOs: 51 to 52 are substituted by another amino acid. Alternatively or in addition, the N-terminal capping module comprised in the modified repeat domain generated by the method of the invention comprises a sequence selected from the group consisting of (1) SEQ ID NO: 45 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in SEQ ID NO: 45 are substituted by another amino acid. Alternatively or in addition, the C-terminal capping module comprised in the modified repeat domain generated by the method of the invention comprises a sequence selected from the group consisting of (1) SEQ ID NO: 56 and (2) sequences in which up to 9 amino acids, up to 8 amino acids, up to 7 amino acids, up to 6 amino acids, up to 5 amino acids, up to 4 amino acids, up to 3 amino acids, up to 2 amino acids, or up to 1 amino acid in SEQ ID NO: 56 are substituted by another amino acid.

In some embodiments, each internal repeat module comprised in the modified repeat domain generated by the method of the invention comprises one or more residues selected from the group consisting of (i) Gln in position 1, and (ii) Glu in position 26, numbered relative to SEQ ID NO: 48.

In some embodiments, the N-terminal capping module comprised in the modified repeat domain generated by the method of the invention comprises one or more residues selected from the group consisting of (i) Thr in position 4, (ii) Gln in position 5, (iii) Leu, Val, Ile, Ala or Met in position 15, and (iv) Gln or Ala in position 23, numbered relative to SEQ ID NO: 43.

In some embodiments, the C-terminal capping module comprised in the modified repeat domain generated by the method of the invention comprises one or more residues selected from the group consisting of (i) Thr or Asn in position 3, (ii) Ser in position 4, (iii) Thr or Ser in position 6, (iv) Asn in position 17, (v) Glu in position 18, and (vi) Asp in position 19, numbered relative to SEQ ID NO: 54.

Accordingly, in an exemplary embodiment, the modified repeat domain generated by the method of the invention comprises (1) an N-terminal capping module comprising Thr in position 4, Gln in position 5, Leu in position 15 and Ala in position 23, numbered relative to SEQ ID NO: 43, (2) two internal repeat modules each comprising a Glu in position 26, numbered relative to SEQ ID NO: 48, and (3) a C-terminal capping module comprising Thr in position 3, Ser in position 4, Ser in position 6, Asn in position 17, Glu in position 18 and Asp in position 19, numbered relative to SEQ ID NO: 54.

In another exemplary embodiment, the modified repeat domain generated by the method of the invention comprises (1) an N-terminal capping module comprising Thr in position 4, Gln in position 5, Leu in position and Gln in position 23, numbered relative to SEQ ID NO: 43, (2) two internal repeat modules each comprising Gln in position 1 and Glu in position 26, numbered relative to SEQ ID NO: 48, and (3) a C-terminal capping module comprising Asn in position 17, Glu in position 18 and Asp in position 19, numbered relative to SEQ ID NO: 54.

In further embodiments, the modified repeat domain generated by the method of the invention comprises a sequence selected from the group consisting of (1) SEQ ID NOs: 104 to 114 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity among the framework residues of any one of SEQ ID NOs: 104 to 114. In some embodiments, the modified repeat domain generated by the method of the invention comprises a sequence selected from the group consisting of (1) SEQ ID NOs: 104 and 112 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity among the framework residues of any one of SEQ ID NOs: 104 and 112. In other embodiments, the modified repeat domain generated by the method of the invention comprises a sequence selected from the group consisting of (1) SEQ ID NO: 104 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity among the framework residues of SEQ ID NO: 104 are substituted by another amino acid.

In a particular embodiment, the modified repeat domain generated by the method of the invention comprises a sequence selected from the group consisting of (1) SEQ ID NOs: 2 to 5, 8 to 10, 12 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 2 to 5, 8 to 10, 12 and 95 to 98. Thus, in a more particular embodiment, said modified designed repeat domain comprises an amino acid sequence selected from the group consisting of (1) SEQ ID NOs: 2 to 5, 8 to 10, 12 and 95 to 98 and (2) sequences with at least 80% amino acid sequence identity with any one of SEQ ID NOs: 2 to 5, 8 to 10, 12 and 95 to 98.

In a particular embodiment, the modified repeat domain generated by the method of the invention comprises a sequence selected from the group consisting of (1) SEQ ID NOs: 8 to 10, 12 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 8 to 10, 12 and 95 to 98. Thus, in a more particular embodiment, said modified designed repeat domain comprises an amino acid sequence selected from the group consisting of (1) SEQ ID NOs: 8 to 10, 12 and 95 to 98 and (2) sequences with at least 80% amino acid sequence identity with any one of SEQ ID NOs: 8 to 10, 12 and 95 to 98.

In a particular embodiment, the modified repeat domain generated by the method of the invention comprises a sequence selected from the group consisting of (1) SEQ ID NOs: 8 to 10 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 8 to 10 and 95 to 98. Thus, in a more particular embodiment, said modified designed repeat domain comprises an amino acid sequence selected from the group consisting of (1) SEQ

ID NOs: 8 to 10 and 95 to 98 and (2) sequences with at least 80% amino acid sequence identity with any one of SEQ ID NOs: 8 to 10 and 95 to 98.

In a particular embodiment, the modified repeat domain generated by the method of the invention comprises a sequence selected from the group consisting of (1) SEQ ID NOs: 8 to 9 and 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 8 to 9 and 95 to 98. Thus, in a more particular embodiment, said modified designed repeat domain comprises an amino acid sequence selected from the group consisting of (1) SEQ ID NOs: 8 to 9 and 95 to 98 and (2) sequences with at least 80% amino acid sequence identity with any one of SEQ

ID NOs: 8 to 9 and 95 to 98.

In a particular embodiment, the modified repeat domain generated by the method of the invention comprises a sequence selected from the group consisting of (1) SEQ ID NOs: 95 to 98 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 95 to 98. Thus, in a more particular embodiment, said modified designed repeat domain comprises an amino acid sequence selected from the group consisting of (1) SEQ ID NOs: 95 to 98 and (2) sequences with at least 80% amino acid sequence identity with any one of SEQ ID NOs: 95 to 98.

In a particular embodiment, the modified repeat domain generated by the method of the invention comprises a sequence selected from the group consisting of (1) SEQ ID NO: 12 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with SEQ ID NO: 12. Thus, in a more particular embodiment, said modified designed repeat domain comprises an amino acid sequence selected from the group consisting of (1) SEQ ID NO: 12 and (2) sequences with at least 80% amino acid sequence identity with SEQ ID NO: 12.

In a particular embodiment, the modified repeat domain generated by the method of the invention comprises a sequence selected from the group consisting of (1) SEQ ID NOs: 2 to 5 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ

ID NOs: 2 to 5. Thus, in a more particular embodiment, said modified designed repeat domain comprises an amino acid sequence selected from the group consisting of (1) SEQ ID NOs: 2 to 5 and (2) sequences with at least 80% amino acid sequence identity with any one of SEQ ID NOs: 2 to 5.

In a particular embodiment, the designed ankyrin repeat domain generated by the method of the invention comprises a sequence selected from the group consisting of (1) SEQ ID NOs: 2, 3 and 5 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with any one of SEQ ID NOs: 2, 3 and 5. Thus, in a more particular embodiment, said modified designed repeat domain comprises an amino acid sequence selected from the group consisting of (1) SEQ ID NOs: 2, 3 and 5 and (2) sequences with at least 80% amino acid sequence identity with any one of SEQ ID NOs: 2, 3 and 5.

In further embodiments, the designed ankyrin repeat domain generated by the method of the invention comprises one or more residues selected from the group consisting of (i) Thr in position 4, (ii) Gln in position 5, (iii) Leu, Val, Ile, Ala or Met in position 15, (iv) Gln or Ala in position 23, (v) Gln in position 31, (vi) Glu in position 56, (vii) Gln in position 64, (viii) Glu in position 89, (ix) Thr or Asn in position 99, (x) Ser in position 100, (xi) Thr or Ser in position 102, (xii) Asn in position 113, (xiii) Glu in position 114, and (xiv) Asp in position 115, numbered relative to SEQ ID NO: 6.

Accordingly, in an exemplary embodiment, the designed ankyrin repeat domain generated by the method of the invention comprises Thr in position 4, Gln in position 5, Leu in position 15, Ala in position 23, Glu in position 56, Glu in position 89, Thr in position 99, Ser in position 100, Ser in position 102, Asn in position 113, Glu in position 114 and Asp in position 115, numbered relative to SEQ ID NO: 6.

In another exemplary embodiment, the designed ankyrin repeat domain generated by the method of the invention comprises Thr in position 4, Gln in position 5, Leu in position 15, Gln in position 23, Gln in position 31, Glu in position 56, Gln in position 64, Glu in position 89, Asn in position 113, Glu in position 114 and Asp in position 115, numbered relative to SEQ ID NO: 6.

In some embodiments, the modified repeat domain generated by the method of the invention is linked to a drug moiety. In some embodiments, the drug moiety is linked covalently or non-covalently to the modified repeat domain generated by the method of the invention. In a more particular embodiment, the drug moiety is linked to the modified repeat domain generated by the method of the invention by a chelator. Appropriate chelators may be selected depending on the application of said modified repeat domain of the invention. In a particular embodiment, said chelator is DTPA. In a more particular embodiment, said drug moiety is a radionuclide. The choice of said radionuclide may depend on the intended application of the modified repeat domain generated by the method of the invention (e.g. diagnostic vs. therapeutic).

In a particular embodiment, said drug moiety is a therapeutic moiety. In one embodiment, said therapeutic moiety is a toxin. In one embodiment, said therapeutic moiety is a radionuclide. In a more particular embodiment, said radionuclide is indium-111. In another particular embodiment, said drug moiety is a diagnostic moiety. In a more particular embodiment, said diagnostic moiety is a fluorophore, a chromophore, an imaging agent or a radionuclide.

In preferred embodiments, the modified repeat domain generated by the method of the invention is a modified designed ankyrin repeat domain.

Recombinant Proteins

In one aspect, the invention provides a recombinant protein comprising the designed repeat domain of the invention.

The designed repeat domains of the invention can be genetically fused to further components, such as, e.g., a drug moiety, a protein or an agent, and such fusions are also referred to as “recombinant protein”. Linkers known in the art may be used between repeat domains in such repeat proteins (see, e.g., WO 2021/116469) or between a repeat domain and said further component. Such recombinant proteins are in particular envisioned for use in medicine.

In some embodiments, the recombinant protein of the invention comprises a repeat domain of the invention which is linked to a drug moiety. In some embodiments, additional drug moieties may be linked to such recombinant proteins. In other embodiment, the recombinant protein of the invention comprises a repeat domain of the invention which is not linked to a drug moiety. In more particular embodiments, one or more drug moieties may be linked to said recombinant protein.

In some embodiments, the recombinant proteins of the invention comprise one or more additional designed ankyrin repeat domains.

Nucleic Acids, Vectors and Host Cells In another aspect, the invention relates to an isolated nucleic acid encoding the amino acid sequence of the designed repeat domain of the invention or of the recombinant protein of the invention. In one embodiment, the invention relates to an isolated nucleic acid encoding the amino acid sequence of the recombinant protein of the present invention. In one embodiment, the invention relates to an isolated nucleic acid encoding the amino acid sequence of the designed repeat domain of the present invention.

Furthermore, the invention relates to vectors comprising any nucleic acid of the invention. Accordingly, in another aspect, the invention provides a recombinant expression vector comprising a nucleic acid according to the invention, wherein the vector optionally comprises an expression control sequence, allowing expression in prokaryotic or eukaryotic host cells of the encoded polypeptide, operably linked to said nucleic acid. The nucleic acid sequence can be inserted in the recombinant vector by methods well known to a person skilled in the art such as, for example, those that are described in MOLECULAR CLONING: A LABORATORY MANUAL, Sambrook et al, 4th Ed., Cold Spring Harbor Laboratory Press, Cold Spring Harbor, N Y., 2001.

Nucleic acids are well known to the skilled person in the art. Nucleic acids were used to produce designed ankyrin repeat domains or recombinant binding proteins of the invention in E. coli, e.g. as further described 35 in U.S. Pat. No. 7,417,130.

In another aspect, the invention provides a host cell comprising a recombinant expression vector according to the invention. The host cell can be, for example, bacterial cells such as Escherichia coli or Streptomyces, fungal cells such as Aspergillus and yeasts such as Saccharomyces, insect cells, mammalian cells such as Chinese Hamster Ovary (CHO) cells, CI 27 mouse cell line, BHK cell line of Syrian hamster cells, Human Embryonic Kidney 293 (HEK 293) cells. In some embodiment, the host cell is a CHO cell ora HEK 293 cell. The host cells can be used, for example, to express a recombinant protein of the invention.

Compositions

The invention further relates to pharmaceutical compositions comprising one or more of a designed ankyrin repeat domain, a recombinant protein, a nucleic acid and/or a recombinant expression vector described herein and a pharmaceutically acceptable carrier or diluent. The invention also relates to uses and methods of treatment and diagnosis using said pharmaceutical compositions disclosed herein. The methods and uses encompassed by the present invention are described in more detail below.

The pharmaceutical compositions described herein may be prepared using methods known in the art.

The pharmaceutical compositions optionally comprise a pharmaceutically acceptable carrier or excipient or diluent. Standard pharmaceutical carriers include a phosphate buffered saline solution, water, emulsions such as an oil/water or water/oil emulsion, and various types of wetting agents.

The pharmaceutical compositions of the invention may comprise any other pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, colouring agents, desiccants, detergents, diluents, disinfectants, disintegrants, dispersing agents, dissolution enhancing agents, dyes, emollients, emulsifying agents, emulsion stabilizers, fillers, film forming agents, flavour enhancers, flavouring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Macadk Publishing Co., Easton, Pa., 1980), which is incorporated by reference in its entirety.

In one embodiment, the invention provides a pharmaceutical composition comprising one or more of: (i) a designed ankyrin repeat domain according to the invention, (ii) a recombinant protein according to the invention, (iii) a nucleic acid according to the invention, and/or (iv) a recombinant expression vector according to the invention, and optionally at least one pharmaceutically acceptable carrier or diluent.

Therapeutic and Diagnostic Uses and Methods According to the Invention In the context of the invention, the terms “medical condition”, “disease” and “disorder” are used interchangeably and include but are not limited to cancer. In one preferred embodiment, said medical condition is a cancer.

In one aspect, the invention provides a method of treating and/or diagnosing a medical condition, the method comprising the step of administering to a patient in need thereof a therapeutically and/or diagnostically effective amount of the designed repeat domain of the invention, the recombinant protein of the invention, the nucleic acid of the invention or the pharmaceutical composition of the invention.

Further provided is the designed repeat domain, the recombinant protein, the nucleic acid, or the pharmaceutical composition of the invention for use in a method of treating and/or diagnosing a medical condition.

In one embodiment, the invention relates to the use of the designed repeat domain, the recombinant protein, the nucleic acid, or the pharmaceutical composition according to the present invention for the treatment and/or diagnosis of a disease. For that purpose, the designed repeat domain, the recombinant protein, the nucleic acid, or the pharmaceutical composition according to the present invention is administered to a patient in need thereof, in a therapeutically and/or diagnostically effective amount.

In one embodiment, the invention relates to a method of treatment and/or diagnosis of a medical condition, the method comprising the step of administering, to a patient in need of such a treatment, a therapeutically and/or diagnostically effective amount of the designed repeat domain, recombinant protein, nucleic acid or pharmaceutical composition of the invention.

In one embodiment, the invention relates to the use of the designed repeat domain, recombinant protein, nucleic acid or pharmaceutical composition of the present invention for the treatment and/or diagnosis of a medical condition.

In one embodiment, the invention relates to the designed repeat domain, recombinant protein, nucleic acid or pharmaceutical composition of the invention for use in the treatment and/or diagnosis of a medical condition. In one embodiment, the invention relates to the use of the designed repeat domain, recombinant protein, nucleic acid or pharmaceutical composition of the invention, as a medicament for the treatment and/or diagnosis of a medical condition.

In one embodiment, the invention relates to a process of treatment and/or diagnosis of a medical condition using the designed repeat domain, recombinant protein, nucleic acid or pharmaceutical composition of the invention. In one embodiment, the invention relates to a process for the manufacturing of a medicament for the treatment and/or diagnosis of a medical condition, wherein the designed repeat domain, recombinant protein, nucleic acid or pharmaceutical composition of the invention is an active ingredient of the medicament.

In one embodiment, the invention relates to the use of the designed repeat domain, recombinant protein, nucleic acid or pharmaceutical composition of the invention, for manufacturing of a medicament.

In one embodiment, the invention relates to the use of the designed repeat domain, recombinant protein, nucleic acid or pharmaceutical composition of the invention, for manufacturing of a medicament for the treatment and/or diagnosis of a medical condition.

In a further embodiment, the invention relates to the use of the designed repeat domain, recombinant protein, nucleic acid or pharmaceutical composition of the invention for the manufacture of a medicament that is used for the treatment and/or diagnosis of a medical condition, preferably a neoplastic disease, more preferably cancer.

In some preferred embodiments, said medical condition is a cancer.

In some embodiments, said patient is a mammal. In preferred embodiments, the patient is a human.

In some embodiments, a single administration of said designed ankyrin repeat domain, recombinant protein, nucleic acid or pharmaceutical composition of the invention may be sufficient. In other embodiments, repeated administration may be necessary. Various factors will impact on the number and frequency of administrations, such as the age and general health of the subject, as well as the nature and typical dosage regime of any drug moiety comprised in such designed ankyrin repeat domain, recombinant protein, nucleic acid or pharmaceutical composition of the invention.

The designed repeat domain, recombinant protein, nucleic acid or pharmaceutical compositions described herein may be used in combination with another therapeutic and/or diagnostic agent. Each therapeutic and/or diagnostic agent may be administered simultaneously (e.g., in the same medicament or at the same time), concurrently (i.e., in separate medicaments administered one right after the other in any order) or sequentially in any order. Sequential administration may be useful when the therapeutic or diagnostic agents in the combination therapy and/or diagnosis are in different dosage forms (e.g., one agent is a tablet or capsule and another agent is a sterile liquid) and/or are administered on different dosing schedules, e.g., an analgesic that is administered at least daily and a biotherapeutic that is administered less frequently, such as once weekly or once every two weeks.

Administration may include topical administration, oral administration, and parenteral administration. The typical route of administration is parenteral administration.

In particular embodiments, oral administration is excluded from any of the above method or uses according to the invention. Accordingly, in some particular embodiment, the designed repeat domain of the invention, the recombinant protein of the invention, the nucleic acid of the invention or the pharmaceutical composition of the invention for use in a method of treating and/or diagnosing a medical condition according to the invention is not administered orally. In some embodiment, the invention provides a method of treating and/or diagnosing a medical condition comprising the step of administering to a patient in need thereof a therapeutically and/or diagnostically effective amount of the designed repeat domain of the invention, the recombinant protein of the invention, the nucleic acid of the invention or the pharmaceutical composition of the invention, and wherein said administration is not an oral administration.

In some further embodiments, the designed repeat domain, recombinant protein or pharmaceutical compositions described herein may be used in radiopharmaceutical therapy or diagnostics. Exemplary approaches and indications are for instances disclosed in Sgouros, George, et al. “Radiopharmaceutical therapy in cancer: clinical advances and challenges.” Nature Reviews Drug Discovery 19.9 (2020): 589-608.

In some alternative embodiments, the designed repeat domain, recombinant protein or pharmaceutical compositions described herein may be used in therapeutic and/or diagnostic approaches for which also antibody-drug conjugates may be used. Such approaches and indications are for instance disclosed in Drago, Joshua Z., Shanu Modi, and Sarat Chandarlapaty. Nature Reviews Clinical Oncology 18.6 (2021) and Tarantino, Paolo, et al., CA: a cancer journal for clinicians 72.2 (2022): 165-182.

The invention is not restricted to the particular embodiments described in the Examples.

### EXAMPLES

Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA). TEV protease was from Sigma-Aldrich (USA). Double-stranded gene fragments (eBlocks) were obtained from IDT (US). Maleimide DTPA was purchased from Chematech, metal-free PBS was purchased from VWR and Chelex 100 chelating resins were purchased from BioRad.

Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit.. By such methods designed ankyrin repeat protein libraries having randomized ankyrin repeat modules and/or randomized capping modules can be constructed. For example, such libraries could accordingly be assembled based on a fixed N-terminal capping module or a randomized N-terminal capping module, one or more randomized repeat modules, and a fixed C-terminal capping module or a randomized C-terminal capping module (see, e.g., the N-terminal capping modules and C-terminal capping modules provided in WO2021116462 and WO2021116469. Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g. 1 to 20 amino acids) inserted in one or more beta-turns of an ankyrin repeat domain.

An N-terminal capping module of an ankyrin repeat protein library preferably possesses the RILLAA, RILLKA or RELLKA motif and any such C-terminal capping module of an ankyrin repeat protein library preferably possesses the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V20, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

The experimental conditions for some examples are also further described in WO2012069654, WO2016156596 and WO2021116462.

### Example 1: Construction, Expression and Purification of DARPins Variants

DARPins having a defined amino acid sequence can be produced by gene synthesis of a corresponding reverse translated nucleic acid sequence, subcloning into an appropriate expression vector of an expression system (e.g. an E. coli expression system), expression and purification of the protein. Such methods are known to the person skilled in the art.

Charge-engineered DARPin variants were constructed and investigated by assessing which amino acid positions would allow a charge reducing substitution of a parental amino acid residue while still retaining favorable biophysical properties and/or biological activity (binding to target). Certain amino acid substitutions have been studied in Simeon, R. A., et al., Protein Engineering, Design and Selection, 34, (2021) for the purpose of increasing protease stability of bacterial exotoxin-specific DARPins.

Overall, a set of 9 DARPin variants bearing different numbers of positively and negatively charged amino acids residues was designed. These variants and their parental DARPins were subsequently produced and characterized as described in the following paragraphs.

The three different parental DARPins chosen as starting points for engineering the DARPin charge-variants are DARPin01 (SEQ ID NO: 1), DARPin06 (SEQ ID NO: 6) and DARPin11 (SEQ ID NO: 11). Parental DARPin06 and DARPin11 were described previously in WO2018054971 and parental DARPin01 in WO2020245746 & WO2020245175. DARPin07 was designed to be subsequently used as a negative control; DARPin07 has a higher pI and higher percentage of basic amino acids among the framework residues as compared to its parental DARPin06. Details about the generated DARPin variants and parental DARPins are shown in Table 6 and FIGS. 1A-1B.

TABLE 6

| | | | | | KR/DE ratio | |
|---|---|---|---|---|---|---|
| | | | KR/DE ratio | KR/DE | (framework | |
| | | | (all | ratio (all | residues | |
| | | | residues, | residues, | only, in | |
| | | Isoelectric | in entire | in N-cap | entire | |
| Sequence | Name | point (pl) | DARPin) | only) | DARPin) | Type |
| SEQ ID NO: 1 | DARPin01 | 4.93 | 0.64 | 0.71 | 0.42 | Parent |
| SEQ ID NO: 2 | DARPin02 | 3.75 | 0.08 | 0.20 | 0.11 | Variant |
| SEQ ID NO: 3 | DARPin03 | 4.05 | 0.19 | 0.50 | 0.25 | Variant |
| SEQ ID NO: 4 | DARPin04 | 4.46 | 0.42 | 0.50 | 0.33 | Variant |
| SEQ ID NO: 5 | DARPin05 | 4.04 | 0.22 | 0.20 | 0.22 | Variant |
| SEQ ID NO: 6 | DARPin06 | 4.65 | 0.52 | 0.71 | 0.37 | Parent |
| SEQ ID NO: 7 | DARPin07 | 7.10 | 1.00 | 0.75 | 0.89 | Variant |
| | | | | | | (neg. control) |
| SEQ ID NO: 8 | DARPin08 | 4.26 | 0.30 | 0.50 | 0.17 | Variant |
| SEQ ID NO: 9 | DARPin09 | 4.50 | 0.30 | 0.50 | 0.25 | Variant |
| SEQ ID NO: 10 | DARPin10 | 4.88 | 0.54 | 0.40 | 0.45 | Variant |
| SEQ ID NO: 11 | DARPin11 | 4.64 | 0.45 | 0.67 | 0.42 | Parent |
| SEQ ID NO: 12 | DARPin12 | 4.53 | 0.36 | 0.67 | 0.27 | Variant |

The DNA encoding each of the designed ankyrin repeat domains of SEQ ID NOs: 1 to 12 was cloned into a pQE (QIAgen, Germany) based expression vector providing an N-terminal 6xHis-tag to facilitate simple protein purification. Proteins comprising one of SEQ ID NOs: 1 to 12, respectively, and additionally having a His-TEV tag (SEQ ID NO: 13) fused to their N-termini and a GSGSC tag (SEQ ID NO: 14) fused to their C-termini were expressed in E. coli, and purified over an IMAC column before desalting over a HiLoad 26/600 Superdex 200 column. Main fractions were pooled and 4 mL of DARPin (different concentrations) were digested with 290 μL of TEV (Sigma Aldrich, >3kU/mg TEV) at RT (for 2 h), then at 4° C. (overnight). Samples were taken after 2 h and after overnight digestion and analyzed on an SDS-PAGE to assess the degree of cleavage. Non-cleaved DARPins still containing the His-tag as well as the His-tagged TEV protease were removed by incubating for 2 h with 5 mL of IMAC resin on a roller shaker, before centrifugation and removal of the IMAC resins by decanting and filtration. Supernatant/flow-through was purified over a size-exclusion chromatography step, before up-concentration. Final purified samples were stored in PBS. Detailed methods for the production and purification of proteins are well known to the practitioner in the art.

### Example 2: Size-Exclusion Chromatography Analysis

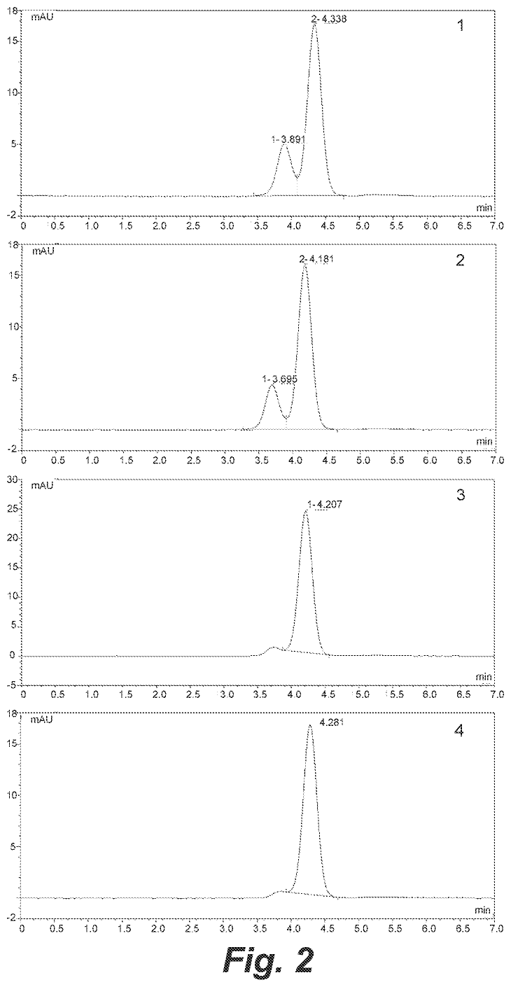

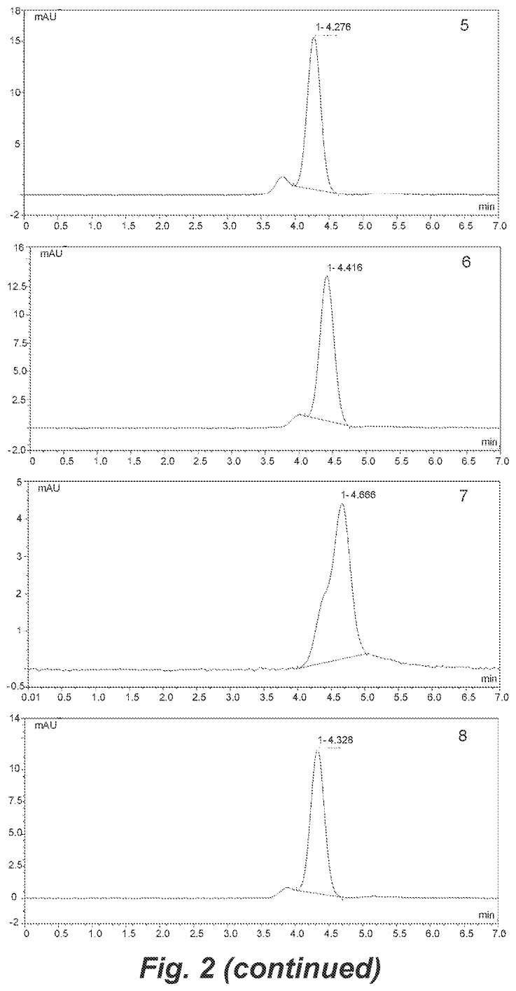

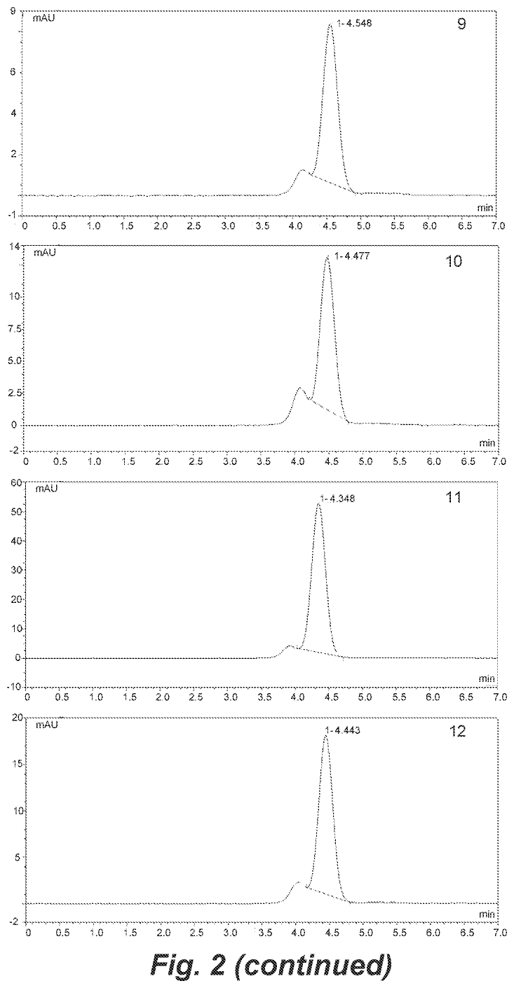

Samples of Example 1 were analyzed on a GE Superdex Increase 200 150/5 column on an Agilent 1200 HPLC system in PBS at 0.5 ml/min flow rate. Of each protein, 0.1 ml at 100 micromolar concentration were analyzed. Proteins comprising one of SEQ ID NO: 1 to 12, respectively, and a C-terminal GSGSC tag (SEQ ID NO: 14) elute as partially dimeric peaks due to oxidation of the C-terminal cysteine to form homo-dimers. At least 95% of the area under the curve corresponded to the combined monomer+dimer fraction. Results are shown in FIG. 2. These results indicate that the proteins are biophysically well-behaved.

### Example 3: DTPA Coupling to C-Terminal Cysteine of DARPin and ¹¹¹in Loading

His-tag free DARPins containing the C-terminal Cys were first reduced by incubating a protein solution of approximatively 5 mg/mL with a 10-fold excess of 0.5 M TCEP (pH adjusted to 7.6). The reaction was shaken for 4 h at room temperature. Subsequently, reduced DARPin solution was mixed with 0.5 M EDTA at a 1:1 molar ratio and stirred for 15 min. Then, a 5-fold molar excess of 50 mM maleimide-DTPA (2,2′-(1-carboxy-2-(carboxymethyl)-13-(2,5-dioxo-2,5-dihydro-1H-pyrrol-1-yl)-10-oxo-2,5,8,11-tetraazatridecane-5,8-diyl)diacetic acid) dissolved in DMSO was added and stirred for 1 h at room temperature. Probes of the sample were taken and used for analysis via ESI-MS to assess the coupling efficiency of the reaction. If coupling efficiency was >90%, samples were desalted over PD-10 column according to the manufacturer's instruction into metal-free PBS. Then, the protein solution was concentrated and diluted with metal-free PBS for three times over Amicon Ultra-15 centrifugal filters (3K) to desalt the protein further. The final concentration was determined by UV-absorption and a probe for another ESI-MS analysis was taken and measured.

To load the DTPA-coupled DARPin with the radioisotope indium-111, 10⁻²⁰ MBq of 111 In was mixed with 5 μL of 1 M ammonium acetate buffer, then the DTPA-coupled DARPin was added (50-200 μL of approximatively 1 mg/mL DARPin-DTPA construct). The solution was stirred at 37.5° C. for 18 hours, then 1 μL of 0.5 M EDTA was added to complex non-bound 111 In. The labeling efficiency of ¹¹¹In-labeled DARPin was checked by HPLC, only if the labeling yield was <90%, the labeled protein was purified over a PD-10 desalting column to remove free 111 In. Finally, to reach a specific activity of 7500 Bq/μg of DARPin, non-radioactive loaded DTPA-DARPin was added to adjust for the final concentration. The entire process of generating the radioactively loaded DTPA-coupled DARPins is illustrated in FIG. 3.

### Example 4: SPR Single Trace Analysis

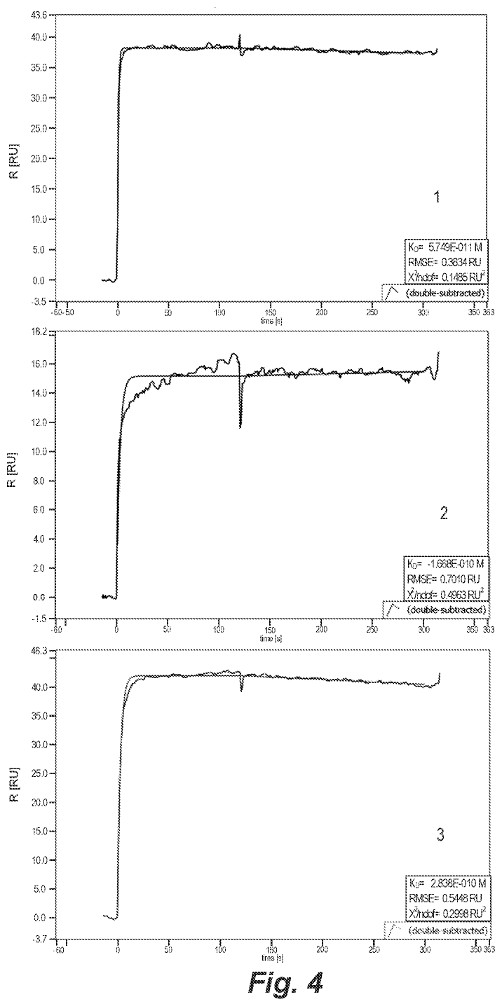

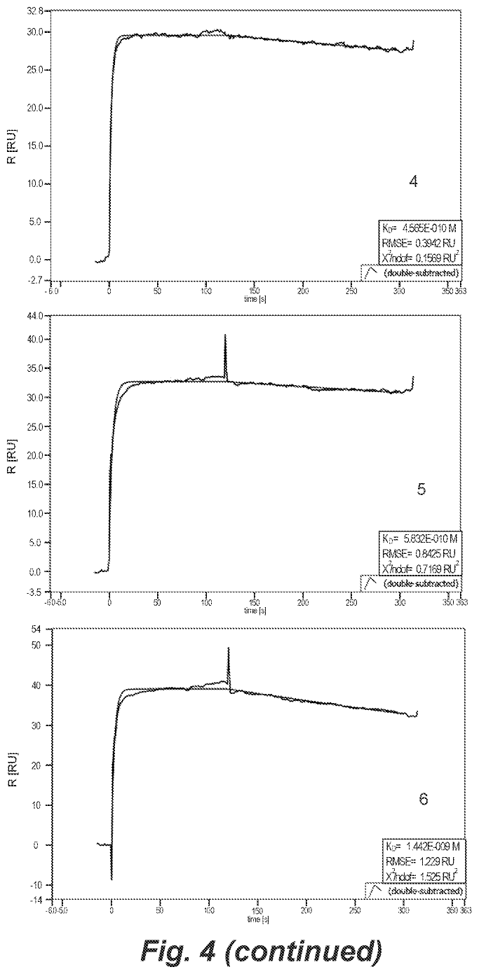

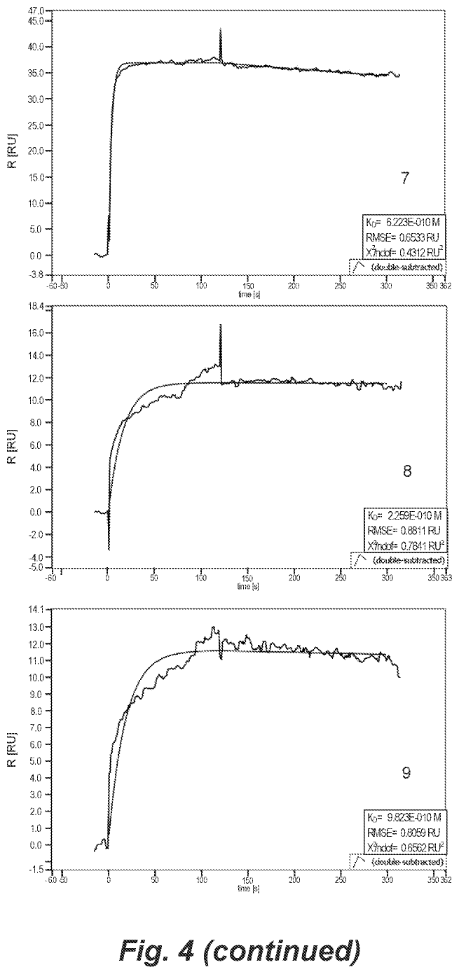

A surface plasmon resonance (SPR) assay was used to determine if the charge-engineered DARPin variants derived from the parental DARPin06 (SEQ ID NO: 6) and DARPin11 (SEQ ID NO: 11) could still bind to HER2. All SPR data were generated using a Bruker Sierra SPR-32 instrument with PBS-T (0.005% Tween 20) as running buffer. A new Bruker BTC Chip was conditioned according to the manufacturer's protocol. The chip was coated with biotinylated target (bio-HER2) to reach a signal intensity of approximatively 600 RU. All analytes (500 nM) were injected in succession for 120s, dissociation was recorded for 180s (25 ul/min). Each injection was followed by a regeneration step with glycine pH 2.0 for 60s. The data was double referenced (control spot and buffer injection) and fitted to a 1:1 Langmuir model. All charge-engineered variants from parental DARPin06 and DARPin11, when coupled to DTPA, still bind the target with a K_{D} lower than 10⁻⁷ M. The DTPA coupled parental binders DARPin06 and DARPin11 were also able to bind their targets. Table 7 shows details of the constructs tested in this SPR assay. A DTPA coupled DARPin is also referred to as a “construct”. SPR curves are shown in FIG. 4 where plots 1 and 2 show the profiles of the HER2-binding parental DARPin06 and DARPin11 (SEQ ID NOs: 6 and 11, respectively). Plots 3, 4, 5, 6 and 7 show the profiles of construct 6 (DARPin06-GSGSC-DTPA), construct 7 (DARPin07-GSGSC-DTPA), construct 8 (DARPin08-GSGSC-DTPA), construct 9 (DARPin09-GSGSC-DTPA) and construct 10 (DARPin10-GSGSC-DTPA), respectively. Plots 8 and 9 show construct 11 (DARPin11-GSGSC-DTPA) and construct 12 (DARPin12-GSGSC-DTPA), respectively. The GSGSC tag (SEQ ID NO: 14) was fused to the C-terminal end of the DARPins.

TABLE 7

| Construct No. | Description | Type | Sequence |
|---|---|---|---|
| 6 | DARPin06-GSGSC-DTPA | Parent | SEQ ID NO: 6 with a C-terminal |
| | | | GSGSC tag of SEQ ID NO: 14 |
| 7 | DARPin07-GSGSC-DTPA | Charge variant | SEQ ID NO: 7 with a C-terminal |
| | | (neg control) | GSGSC tag of SEQ ID NO: 14 |
| 8 | DARPin08-GSGSC-DTPA | Charge variant | SEQ ID NO: 8 with a C-terminal |
| | | | GSGSC tag of SEQ ID NO: 14 |
| 9 | DARPin09-GSGSC-DTPA | Charge variant | SEQ ID NO: 9 with a C-terminal |
| | | | GSGSC tag of SEQ ID NO: 14 |
| 10 | DARPin10-GSGSC-DTPA | Charge variant | SEQ ID NO: 10 with a C-terminal |
| | | | GSGSC tag of SEQ ID NO: 14 |
| 11 | DARPin11-GSGSC-DTPA | Parent | SEQ ID NO: 11 with a C-terminal |
| | | | GSGSC tag of SEQ ID NO: 14 |
| 12 | DARPin12-GSGSC-DTPA | Charge variant | SEQ ID NO: 12 with a C-terminal |
| | | | GSGSC tag of SEQ ID NO: 14 |

### Example 5: Kidney Accumulation Study of ¹¹¹In-Labeled DARPins

This example describes experiments that were performed to investigate the kidney accumulation of DARPins according to the invention, when administered as radiolabeled compounds.

DTPA-coupled DARPins were loaded with indium-111 (¹¹¹In) as described in Example 3, resulting in 12 ¹¹¹In-labeled DARPins, referred to as radiolabeled DARPin01 to radiolabeled DARPin12. These radiolabeled DARPins (approximately 150 KBq, 1 mg/kg) were injected into the tail vein of wild-type Balb/c mice (females, 7 weeks of age, CRL).

A total of 54 mice were separated in 12 groups and treated with radiolabeled DARPin01 to 12 as detailed in Table 8. Each radiolabeled DARPin was formulated in PBS+0.05% Tween 20.

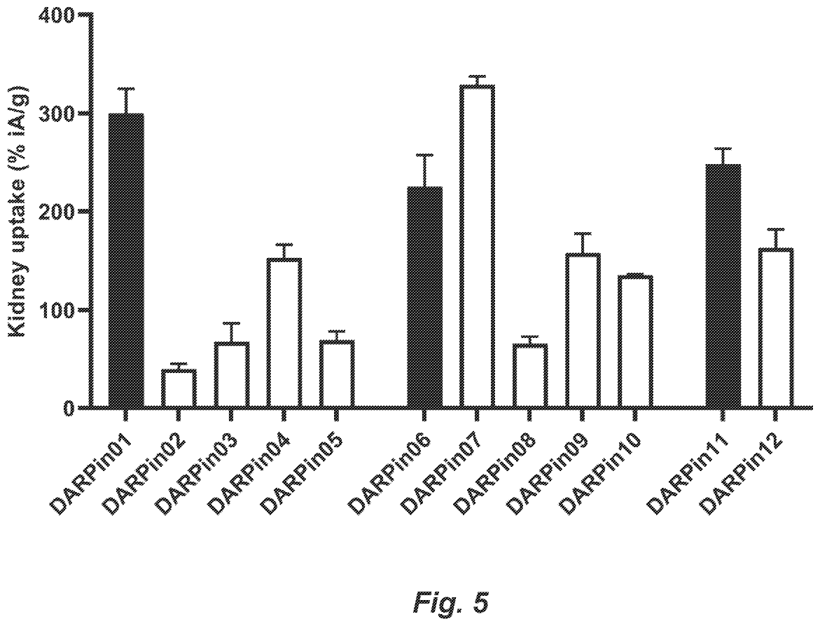

Kidney accumulation was monitored at 4 h post-injection. Mice were euthanized by CO₂ inhalation and cervical dislocation. Kidneys were extracted, weighed and the radioactivity was determined with a γ-counter (Packard Cobra II Gamma D5010, GMI, USA). The data are expressed as injected activity per gram of tissue mass (% IA/g) and shown in FIG. 5.

For autoradiography imaging, kidneys were embedded in OCT and frozen at −80 C. 24 h after collection frozen kidneys sections were prepared on a cryostat and mounted on glass slides. The sections were placed in a X-ray cassette and exposed to phosphor screens for 45 min.

TABLE 8

| | | | Mice | Dosing |
|---|---|---|---|---|
| Group | Treatment | Construct basis | number | mg/kg |
| 1 | radiolabeled DARPin01 | DARPin01-GSGSC-DTPA | 4 | 1 |
| 2 | radiolabeled DARPin02 | DARPin02-GSGSC-DTPA | 4 | 1 |
| 3 | radiolabeled DARPin03 | DARPin03-GSGSC-DTPA | 4 | 1 |
| 4 | radiolabeled DARPin04 | DARPin04-GSGSC-DTPA | 4 | 1 |
| 5 | radiolabeled DARPin05 | DARPin05-GSGSC-DTPA | 4 | 1 |
| 6 | radiolabeled DARPin06 | DARPin06-GSGSC-DTPA | 4 | 1 |
| 7 | radiolabeled DARPin07 | DARPin07-GSGSC-DTPA | 4 | 1 |
| 8 | radiolabeled DARPin08 | DARPin08-GSGSC-DTPA | 4 | 1 |
| 9 | radiolabeled DARPin09 | DARPin09-GSGSC-DTPA | 4 | 1 |
| 10 | radiolabeled DARPin10 | DARPin10-GSGSC-DTPA | 4 | 1 |
| 11 | radiolabeled DARPin11 | DARPin11-GSGSC-DTPA | 4 | 1 |
| 12 | radiolabeled DARPin12 | DARPin12-GSGSC-DTPA | 4 | 1 |

As shown in FIG. 5, the charge-engineered DARPin variants according to the invention (radiolabeled DARPin02, DARPin03, DARPin04, DARPin05, DARPin08, DARPin09, DARPin10 and DARPin12) exhibit a lower renal accumulation of radioactivity compared to the respective parental DARPins (DARPin01, DARPin06 and DARPin11). The main factors influencing renal accumulation in the experiment are the isoelectric point (pI) and the number of basic amino acids (which are positively charged at a physiological pH; in particular Arg and Lys) of the DARPins, either in the entire ankyrin repeat domain or among the framework residues comprised in the ankyrin repeat domain. Radiolabeled DARPin07 was used as negative control and accumulated more than its parental DARPin06. This observation is in accordance with the inventor's hypothesis since DARPin07 has a higher pI and higher percentage of basic amino acid among the framework residues as compared to its parental DARPin06. DARPin07 is not a DARPin according to the present invention.

Table 9 provides the reduction in kidney uptake (in percent) for each variant in comparison to the corresponding parental DARPins:

TABLE 9

| | | Reduction in kidney uptake |
|---|---|---|
| ¹¹¹In-labelled DARPin | Parental | versus parental |
| DARPin02 | DARPin01 | 87% |
| DARPin03 | DARPin01 | 77% |
| DARPin04 | DARPin01 | 49% |
| DARPin05 | DARPin01 | 77% |
| DARPin08 | DARPin06 | 71% |
| DARPin09 | DARPin06 | 30% |
| DARPin10 | DARPin06 | 40% |
| DARPin12 | DARPin11 | 34% |

### Example 6: Biodistribution Study of ¹¹¹In-Labeled DARPins in Tumor Bearing Mice

This example describes experiments that were performed to investigate the tissue accumulation of a selection of DARPins described in Example 5, when administered as radiolabeled compounds to mice bearing HER2-expressing tumors. Two DARPins with binding specificity for HER2 (i.e. DARPin06 and DARPin08) and two DARPins not binding to HER2 (i.e. DARPin01 and DARPin02) where tested in this study.

DARPin01, DARPin02, DARPin06 and DARPin08 were produced, coupled to DTPA and loaded with ¹¹¹In as described in Examples 1 to 4. The resulting radiolabeled DARPins 01, 02, 06 and 08 were injected (approximately 150 KBq, 1 mg/kg bodyweight) into the tail vein of mice bearing HER2-expressing SKOV3ip tumors (females, 9-12 weeks of age, CRL: CD1-Foxn1nu), as further described below. The compounds were formulated in PBS+0.05% Tween 20.

SKOV3ip tumor cells (5×10⁶, in PBS) were implanted subcutaneously into the flank of the mice. Mice were randomized into the different treatment groups (6 animals per group) and i.v. injected with ¹¹¹In-labelled DARPins either two weeks after implantation (tumor volume of approx. 180 mm³) or three weeks after implantation (tumor volume of approx. 360 mm³). Tumors and organs were collected and the % IA/g (equivalent to % ID/g) was determined. Data analysis of the mice injected two weeks after tumor cell implantation and of the mice injected three weeks after tumor cell implantation showed similar results and were therefore pooled for presentation. Table 10 shows a summary of the treatments.

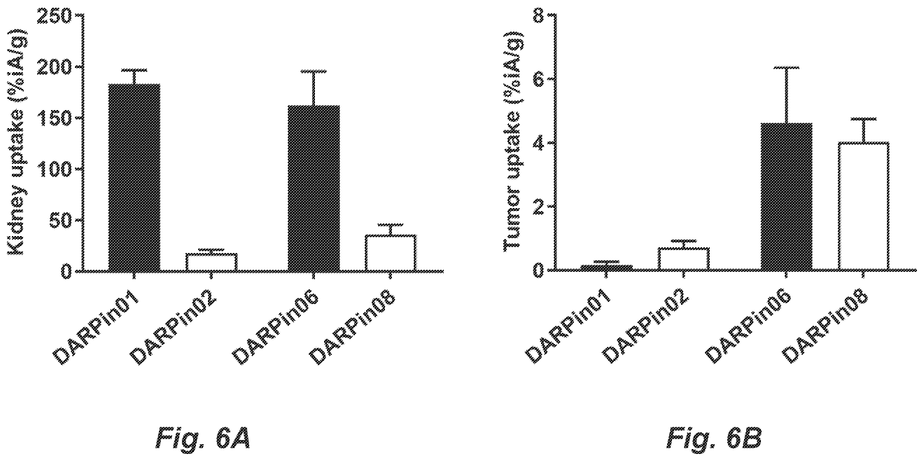

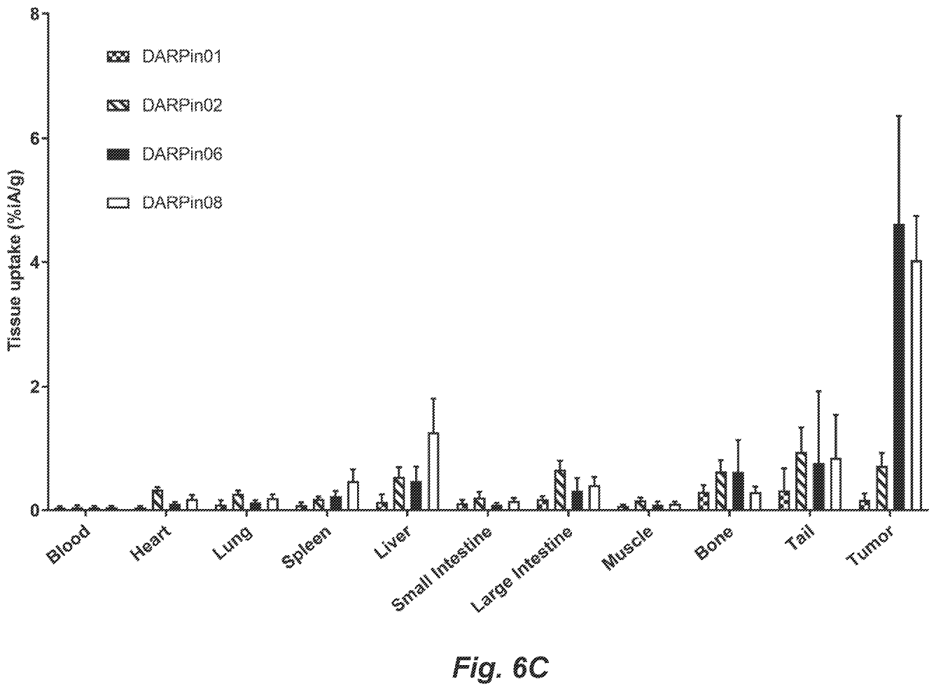

Accumulation in organs and tumor was measured 4 h post-injection. Mice were euthanized by CO₂ inhalation and cervical dislocation. Organs and tumors were extracted, weighed and the radioactivity was determined with a γ counter (Packard Cobra II Gamma D5010, GMI, USA). The data are expressed as mean injected activity per gram of tissue mass (% IA/g) and shown in FIGS. 6A-6C. Error bars show SD.

TABLE 10

| Treatment | Construct details | Mice number | Dosing, mg/kg |
|---|---|---|---|
| radiolabeled DARPin01 | DARPin01-GSGSC-DTPA | 12 | 1 |
| radiolabeled DARPin02 | DARPin02-GSGSC-DTPA | 12 | 1 |
| radiolabeled DARPin06 | DARPin06-GSGSC-DTPA | 12 | 1 |
| radiolabeled DARPin08 | DARPin08-GSGSC-DTPA | 12 | 1 |

As shown in FIG. 6A, the charge-engineered DARPin variants according to the invention used in this experiment (i.e. radiolabeled DARPins 02 and 08) exhibit a lower renal accumulation of radioactivity compared to the respective parental DARPins (i.e. radiolabeled DARPins 01 and 06). The reduction in kidney uptake (in percent) for each of the variants in comparison to the corresponding parental DARPin is shown in Table 11.

TABLE 11

| ¹¹¹In-labelled | ¹¹¹In-labelled | Reduction in kidney uptake |
|---|---|---|
| variant DARPin | parental DARPin | of variant versus parental |
| DARPin02 | DARPin01 | 90% |
| DARPin08 | DARPin06 | 78% |

Accumulation of radioactivity in the tumors and various organs was quantified similarly as for the kidney. The difference in tumor accumulation between the parental DARPins and the engineered variants is negligible, as shown in FIG. 6B. Only very minor unspecific accumulation of the non-binding DARPin01 and DARPin02 was observed in the tumor, while much higher tumor accumulation was observed for the HER2-specific DARPin06 and DARPin08, presumably due to target-specific binding (FIG. 6B).

Table 12 further shows the tumor to kidney ratio measured for the HER2-binding DARPins used in this experiment. The charge engineering performed on DARPin08 provided a 3.9-fold increase of the tumor to kidney ratio compared to the non-engineered parental DARPin06.

TABLE 12

| Samples | Tumor to kidney ratio |
|---|---|
| radiolabeled DARPin06 | 1:35 |
| radiolabeled DARPin08 | 1:9 |

Accumulation of the DARPins in further tissues or organs (blood, heart, lung, spleen, liver, small intestine, large intestine, muscle, bone and tail) was measured and is shown in FIG. 6C. Based on these results, a similarly low accumulation of the engineered variants (DARPin02 and DARPin08) was observed compared to the respective parental DARPins (DARPin01 and DARPin06) in these organs and tissues.

Based on this study, the charge-engineering of DARPins according to the invention does not impair target-specific tumor accumulation of the radiolabeled DARPins, while providing significant reduction of radiolabeled DARPin accumulation in the kidney. Also, charge-engineering of DARPins does not result in significant adverse accumulation in the further analyzed organs.

### Example 7: Time Course Biodistribution of ¹¹¹In-Labeled DARPins in Tumor-Bearing Mice

The retention kinetics of radiolabeled DARPin06 and radiolabeled DARPin08 upon administration in mice bearing HER2-expressing tumors were assessed in a time course biodistribution experiment. Accumulation in tissues of interest (kidney, blood and tumors) was measured 1 h, 4 h, 24 h, 48 h or 96 h after administration.

DARPin06 and DARPin08 used in this study were coupled to DTPA and loaded with Indium-111 as described in Examples 1 to 3.

SKOV3ip tumor cells (5×10⁶, in PBS) were implanted subcutaneously into the flank of CD1-nude mice (Crl:CD1-Foxn1nu mice, 4-5 weeks of age). Mice were randomized into the different treatment groups (4 animals per group) and i.v. injected (single dose) with radiolabeled DARPins at a tumor volume of approx. 350 mm³ (same injected activity and formulations as in Example 6). Tumors and organs were collected and measured 1 h, 4 h, 24 h, 48 h or 96 h after administration following the same protocol as in Example 6. Table 13 shows a summary of treatment and conditions.

TABLE 13

| | Mice | Dosing | Length of | |
|---|---|---|---|---|
| Treatment | number | mg/kg | treatment | |
| radiolabeled DARPin06 | 4 | 1 | 1 | h |
| radiolabeled DARPin06 | 4 | 1 | 4 | h |
| radiolabeled DARPin06 | 4 | 1 | 24 | h |
| radiolabeled DARPin06 | 4 | 1 | 48 | h |
| radiolabeled DARPin06 | 4 | 1 | 96 | h |
| radiolabeled DARPin08 | 4 | 1 | 1 | h |
| radiolabeled DARPin08 | 4 | 1 | 4 | h |
| radiolabeled DARPin08 | 4 | 1 | 24 | h |
| radiolabeled DARPin08 | 4 | 1 | 48 | h |
| radiolabeled DARPin08 | 4 | 1 | 96 | h |

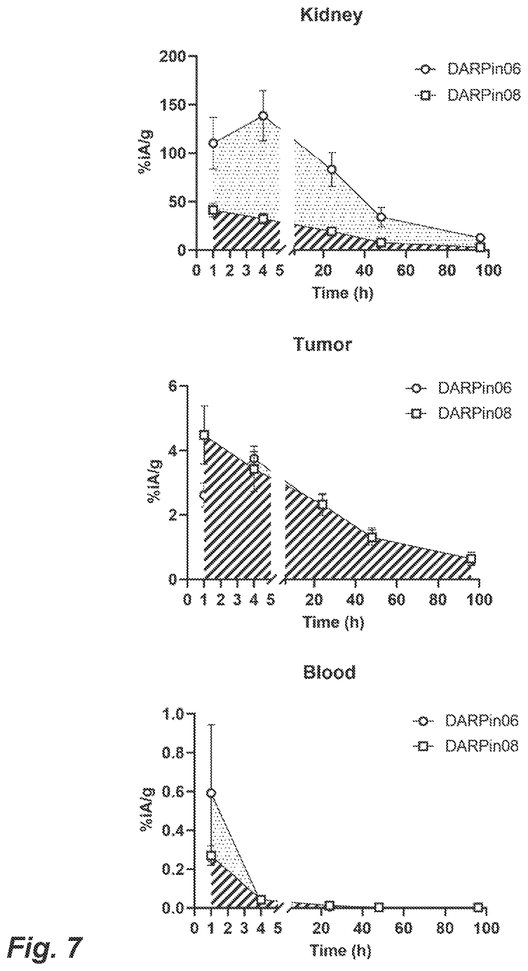

The resulting data are expressed as mean injected activity per gram of tissue mass (% IA/g) and are shown in FIG. 7. Error bars show SD.

The area under the curve (AUC) over the measurement timepoints (1 h to 96 h) and corresponding kidney-to-tumor ratios are shown in Table 14. The kidney accumulation is reduced by 76% (AUC) in the groups treated with engineered DARPin08 as compared to the parental DARPin06 while the tumor accumulation and blood retention remains similar between these groups.

TABLE 14

| | AUC kidney | AUC tumor | Kidney to tumor |
|---|---|---|---|
| Samples | accumulation | accumulation | AUC ratio |
| radiolabeled DARPin06 | 5125 | 151.2 | 33.9 |
| radiolabeled DARPin08 | 1207 | 159.4 | 7.6 |

### Example 8: Design and Biodistribution of Further Charge-Engineered DARPin Variants

Based on the observed reduction in kidney accumulation provided by radiolabeled DARPin08, further charge engineered DARPin variants having binding specificity to HER2 were designed and tested.

Four additional DARPin variants (DARPin13, 14, 15 and 16) were designed and produced following the same methods as in Example 1 and 2. The sequence details are shown in FIGS. 8A-8B and Table 15.

TABLE 15

| | | | KR/DE ratio | KR/DE | KR/DE ratio | |
|---|---|---|---|---|---|---|
| | | | (all | ratio (all | (framework | |
| | | | residues, | residues, | residues only, | |
| | | Isoelectric | in entire | in N-cap | in entire | |
| Sequence | Name | point (pl) | DARPin) | only) | DARPin) | Type |
| SEQ ID NO: 95 | DARPin13 | 4.06 | 0.15 | 0.00 | 0.06 | Variant |
| SEQ ID NO: 96 | DARPin14 | 4.06 | 0.19 | 0.14 | 0.11 | Variant |
| SEQ ID NO: 97 | DARPin15 | 3.99 | 0.15 | 0.50 | 0.11 | Variant |
| SEQ ID NO: 98 | DARPin16 | 4.06 | 0.19 | 0.29 | 0.17 | Variant |

Parental DARPin06, variant DARPin08 and DARPin variants 13-16 were coupled to DTPA. In details, His-tag free DARPins containing the C-terminal Cys were first reduced by incubating a protein solution of approximatively 5 mg/mL with a 10-fold excess of 0.5 M TCEP (pH adjusted to 7.6). The reaction was shaken for 30 min at room temperature. Subsequently, TCEP was removed from the reduced protein by gel-filtration (Zeba spin column, 5 mL). To the purified protein, 0.5 M EDTA was added to a final concentration of 10 mM EDTA and stirred for 15 min. Then, a 5-fold molar excess of 50 mM maleimide-DTPA (2,2′-(1-carboxy-2-(carboxymethyl)-13-(2,5-dioxo-2,5-dihydro-1H-pyrrol-1-yl)-10-oxo-2,5,8,11-tetraazatridecane-5,8-diyl)diacetic acid) dissolved in DMSO was added and stirred for 1 h at room temperature. Coupling efficiency was determined by reverse-phase HPLC (RP-HPLC) and samples were taken forward only if coupling efficiency was >95%. Excess chelator was removed by another gel-filtration step (Zeba spin column, 5 mL) and the protein was re-buffered into metal-free PBS. The final concentration was determined by UV-absorption and a probe for ESI-MS analysis was taken and measured.

Details of the constructs are shown in Table 16. Binding kinetics of the DTPA-coupled DARPin variants were determined as detailed below (section 8.1). Radiolabeled DARPins were subsequently produced by loading 111 In to the constructs following the methods detailed in Example 3. The resulting radiolabeled DARPin13 to 16 as well as radiolabeled DARPin06 and radiolabeled DARPin08 shown in the previous Examples were tested in a mice biodistribution experiment, as further detailed below (section 8.2).

TABLE 16

| Construct No. | Description | Type | Sequence |
|---|---|---|---|
| 6 | as in Table 7 | | |
| 8 | as in Table 7 | | |
| 13 | DARPin13-GSGSC-DTPA | Charge variant | SEQ ID NO: 95 with a C-terminal |
| | | | GSGSC tag of SEQ ID NO: 14 |
| 14 | DARPin14-GSGSC-DTPA | Charge variant | SEQ ID NO: 96 with a C-terminal |
| | | | GSGSC tag of SEQ ID NO: 14 |
| 15 | DARPin15-GSGSC-DTPA | Charge variant | SEQ ID NO: 97 with a C-terminal |
| | | | GSGSC tag of SEQ ID NO: 14 |
| 16 | DARPin16-GSGSC-DTPA | Charge variant | SEQ ID NO: 98 with a C-terminal |
| | | | GSGSC tag of SEQ ID NO: 14 |

8.1 Binding Kinetics

Binding kinetics of the constructs to the target protein HER2 was measured in a multi-trace SPR assay. A new Xantec NAHLC200M chip was conditioned according to the manufacturer's protocol. The chip was coated with biotinylated target (bio-HER2, 10 ug/mL) for 900s to reach a signal intensity of approximately 1200-1300 RU. Three-fold dilutions of the analytes (50, 16.667, 5.556, 1.852 and 0.617 nM) were injected for 300s, dissociation was recorded for 1500s (25 ul/min). Each injection was followed by a regeneration step with glycine pH 2.0 for 60s. The signals (i.e. resonance unit (RU) values) of empty surface and a reference injection (i.e. injection of running buffer only) were subtracted from the RU traces obtained injecting the constructs (double-referencing). Dissociation constants (K_{D}) were calculated from the globally fitted on- and off-rates using standard 1:1-Langmuir model.

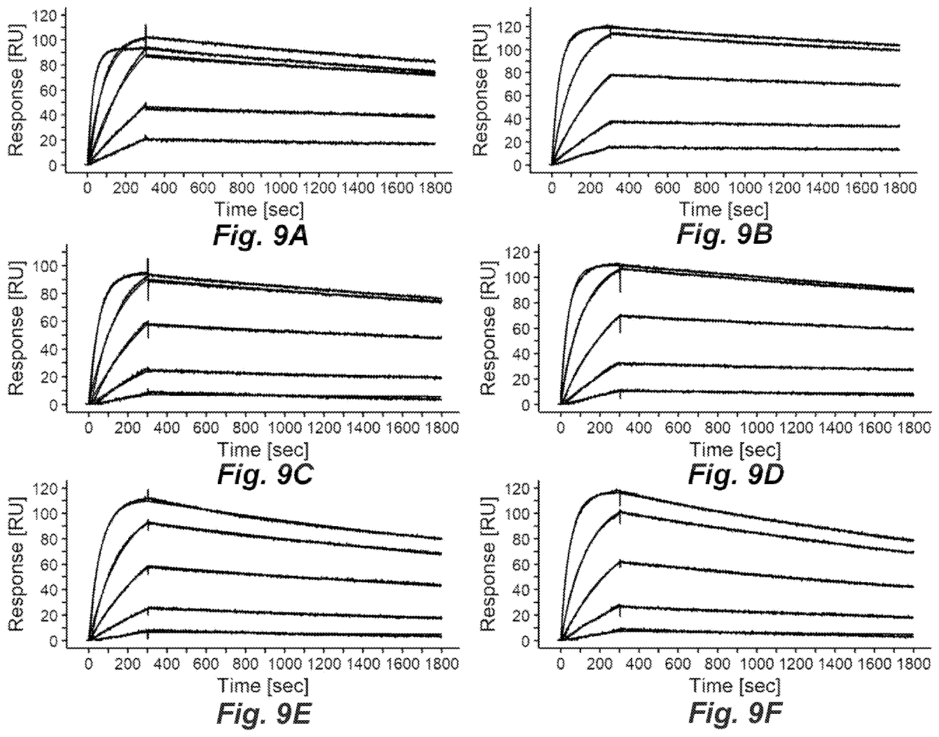

All charge-engineered variants in this experiment, when coupled to DTPA, still bind the target with a K_{D} lower than 10⁻⁹ M. The measured binding characteristics are shown in Table 17 and the SPR curves are shown in FIGS. 9A-9F.

TABLE 17

| Construct No. | kₐ [1/(M · s)] | k_{d} [1/s] | K_{D} [M] |
|---|---|---|---|
| 6 | 6.53E+05 | 1.41E−04 | 2.16E−10 |
| 8 | 5.21E+05 | 8.82E−05 | 1.70E−10 |
| 13 | 3.86E+05 | 1.38E−04 | 3.57E−10 |
| 14 | 5.24E+05 | 1.21E−04 | 2.30E−10 |
| 15 | 3.36E+05 | 2.14E−04 | 6.37E−10 |
| 16 | 4.22E+05 | 2.60E−04 | 6.15E−10 |

8.2 Biodistribution Study of ¹¹¹In-Labeled DARPins in Tumor Bearing Mice

Radiolabeled DARPins 06, 08, 13, 14, 15 and 16 were injected (approximately 150 KBq, 1 mg/kg bodyweight) into the tail vein of mice bearing HER2-expressing SKOV3ip tumors (females, 6-7 weeks of age, CRL: CD1-Foxn1nu), as further described below. The compounds were formulated in PBS+0.05% Tween 20.

SKOV3ip tumor cells (5×10⁶, in PBS) were implanted subcutaneously into the flank of the mice. Mice were randomized into the different treatment groups (4 animals per group) and i.v. injected (single dose) with ¹¹¹In-labelled DARPins at a tumor volume of approx. 350 mm³. Table 18 shows a summary of the treatments.

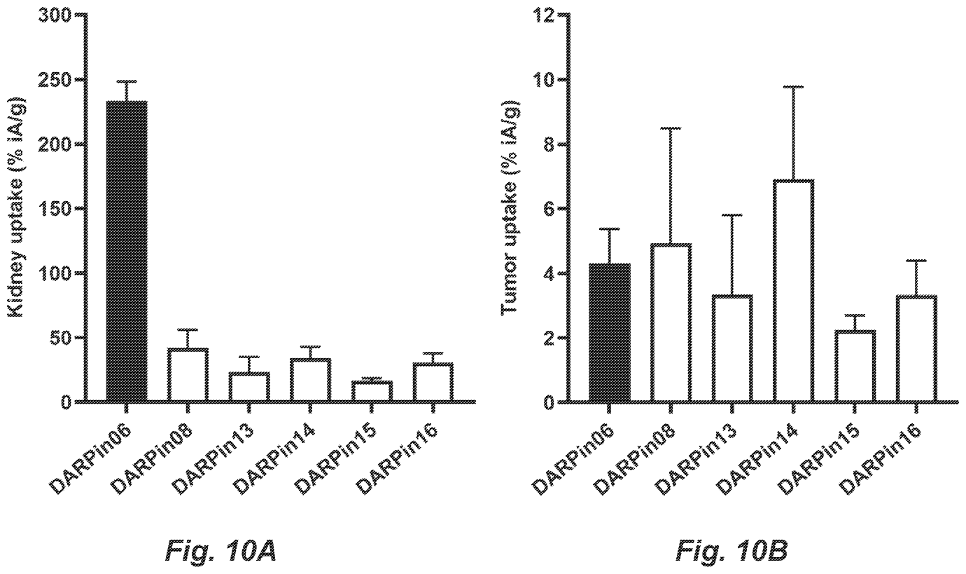

Accumulation in organs and tumor was measured 4 h post-injection. Mice were euthanized by CO₂ inhalation and cervical dislocation. Kidney and tumors were extracted, weighed and the radioactivity was determined with a γ-counter (Packard Cobra II Gamma D5010, GMI, USA). The data are expressed as mean injected activity per gram of tissue mass (% IA/g, equivalent to % ID/g) and are shown in FIGS. 10A-10B (error bars show SD).

TABLE 18

| | | Mice | Dosing |
|---|---|---|---|
| Treatment | Construct basis | number | mg/kg |
| radiolabeled DARPin06 | DARPin06-GSGSC-DTPA | 4* | 1 |
| radiolabeled DARPin08 | DARPin08-GSGSC-DTPA | 4* | 1 |
| radiolabeled DARPin13 | DARPin13-GSGSC-DTPA | 4 | 1 |
| radiolabeled DARPin14 | DARPin14-GSGSC-DTPA | 4 | 1 |
| radiolabeled DARPin15 | DARPin15-GSGSC-DTPA | 4 | 1 |
| radiolabeled DARPin16 | DARPin16-GSGSC-DTPA | 4 | 1 |

*Tumor samples were collected from 2 mice only.

As shown in FIG. 10A, the charge-engineered DARPin variants according to the invention used in this experiment (i.e. radiolabeled DARPins 08 and 13-16) exhibit a lower renal accumulation of radioactivity compared to the parental DARPin (i.e. radiolabeled DARPins 06). The reduction in kidney uptake (in percent) for each of the variants in comparison to the parental DARPin is shown in Table 19.

TABLE 19

| ¹¹¹In-labelled | ¹¹¹In-labelled | Reduction in kidney uptake |
|---|---|---|
| variant DARPin | parental DARPin | of variant versus parental |
| DARPin08 | DARPin06 | 82% |
| DARPin13 | DARPin06 | 90% |
| DARPin14 | DARPin06 | 85% |
| DARPin15 | DARPin06 | 93% |
| DARPin16 | DARPin06 | 87% |

Accumulation of radioactivity in the tumors was quantified similarly as for the kidney. The difference in tumor accumulation between the parental DARPin and the engineered variants is negligible, as shown in FIG. 10B. Table 20 further shows the tumor to kidney ratio measured for the HER2-binding DARPins used in this experiment. The charge engineering performed on DARPins 08, 13, 14, 15 and 16 provided a respective 4.62-fold, 7.44-fold, 9.88-fold, 7.74-fold and 6.03-fold increase of the tumor to kidney ratio compared to the non-engineered parental DARPin06.

TABLE 20

| Samples | Tumor to kidney ratio |
|---|---|
| radiolabeled DARPin06 | 1:57.3 |
| radiolabeled DARPin08 | 1:12.4 |
| radiolabeled DARPin13 | 1:7.7 |
| radiolabeled DARPin14 | 1:5.8 |
| radiolabeled DARPin15 | 1:7.4 |
| radiolabeled DARPin16 | 1:9.5 |

In this study, further charge-engineered DARPins according to the invention have been successfully produced. As also observed in Example 6, this study shows that the charge-engineering of DARPins according to the invention does not impair target-specific tumor accumulation of the radiolabeled DARPins, while providing significant reduction of radiolabeled DARPin accumulation in the kidney. Also, charge-engineering of DARPins does not result in significant adverse accumulation in further analyzed organs.

All autoradiography measurements presented in Examples 5 to 8 were decay-corrected. Data analysis was performed with Prism 9 (GraphPad Software LLC, San Diego, CA, USA).

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific embodiments of the invention described herein. Such equivalents are intended to be encompassed by the following claims.

SEQUENCES

| SEQ | | |
|---|---|---|
| ID NO | Description | Sequence |
| 1 | Designed ankyrin | DLGKKLLEAARAGQDDEVRELLKAGADVNAKDKDGYTPLHLAAREGHLEIVEVLLKAG [symbol] |
| | repeat domain | LHLAAREGHLEIVEVLLKAGADVNAQDKSGKTPADLAADAGHEDIAEVLQKAA |
| 2 | Designed ankyrin | DLGTQLLEAAQAGQLDTVRELLAAGADVNASDNDGYTPLHLAASEGHLEIVEVLLEAG [symbol] |
| | repeat domain | LHLAASEGHLEIVEVLLEAGADVNAQDTSGSTPADLAADAGNEDIAEVLQKAA |
| 3 | Designed ankyrin | DLGSKLLEAAQAGQLDEVRELLKAGADVNAKDNDGYTPLHLAASEGHLEIVEVLLEAG [symbol] |
| | repeat domain | LHLAASEGHLEIVEVLLEAGADVNAQDTSGSTPADLAADAGHEDIAEVLQQAA |
| 4 | Designed ankyrin | DLGSKLLEAAQAGQLDEVRELLKAGADVNAKDKDGYTPLHLAAREGHLEIVEVLLSAG [symbol] |
| | repeat domain | LHLAAREGHLEIVEVLLSAGADVNAQDTSGSTPADLAADAGHEDIAEVLQKAA |
| 5 | Designed ankyrin | DLGTQLLQAAQAGQLDEVRELLAAGADVNAKDNSGYTPLHLAASSGNLEIVEVLLSAG [symbol] |
| | repeat domain | LHLAASSGNLEIVEVLLSAGADVNAQDTSGSTPADLAATAGHEDIAEVLQKAA |
| 6 | Designed ankyrin | DLGKKLLEAARAGQDDEVRELLKAGADVNAKDEYGLTPLYLATAHGHLEIVEVLLKAG [symbol] |
| | repeat domain | HLAAFIGHLEIAEVLLKAGADVNAQDKFGKTPADIAAGAGNEDIAEVLQKAA |
| 7 | Designed ankyrin | DLGTQLLRAAESGQLDTVRTLLKAGADVNAKDEYGLTPLYLATAHGHLQIVRVLLQAG [symbol] |
| | repeat domain | HLAAFIGHLKIAEVLLKAGADVNAQDKFGKTPAQIAATAGNQQIASVLQKAA |
| 8 | Designed ankyrin | DLGTQLLRAAESGQLDEVRELLKAGADVNAQDEYGLTPLYLATAHGHLEIVTVLLEAG [symbol] |
| | repeat domain | HLAAFIGHLEIATVLLEAGADVNAQDKFGKTPADIAAGAGNEDIAEVLQKAA |
| 9 | Designed ankyrin | DLGTQLLQAAESGQLDTVRTLLKAGADVNAQDEYGLTPLYLATAHGHLQIVQVLLSAG [symbol] |
| | repeat domain | HLAAFIGHLQIAQVLLSAGADVNAQDKFGTTPAQIAATAGNQQIASVLQQAA |
| 10 | Designed ankyrin | DLGTQLLQAAESGQLDTVRELLKAGADVNAQDEYGLTPLYLATAHGHLQIVRVLLQAG [symbol] |
| | repeat domain | HLAAFIGHLEIATVLLKAGADVNAQDKFGKTPADIAATAGNQQIASVLQKAA |
| 11 | Designed ankyrin | DLGIKLLFAAAKGQDDEVRELLKAGADVNAKDFQGVTPLHIAAQSGHLEIVEVLLKAGA [symbol] |
| | repeat domain | HLAAQHGHLEIVEVLLKAGADVNAQDERGWTPADLAADWGHEDIAEVLQKAA |
| 12 | Designed ankyrin | DLGIQLLFAAAKGQLDTVRTLLQAGADVNAKDFQGVTPLHIAAQSGHLQIVEVLLQAGA [symbol] |
| | repeat domain | HLAAQHGHLQIVTVLLQAGADVNAQDERGWTPADLAADWGNQQIAEVLQQAA |
| 13 | Tag | MRGSHHHHHHENLYFQ |
| 14 | Tag | GSGSC |
| 15 | N-terminal | DLGTQLLEAAQAGQLDTVRELLAAGADVNA |
| | capping module | |
| 16 | N-terminal | DLGSKLLEAAQAGQLDEVRELLKAGADVNA |
| | capping module | |
| 17 | N-terminal | DLGTQLLQAAQAGQLDEVRELLAAGADVNA |
| | capping module | |
| 18 | N-terminal | DLGTQLLQAAESGQLDTVRTLLKAGADVNA |
| | capping module | |
| 19 | N-terminal | DLGXZ₁LLXAAXXGQLDZ₂VRZ₃LLZ₄AGADVNA |
| | capping module | wherein Z₁ is selected from the group consisting of Q and K; |
| | | Z₂ is selected from the group consisting of E and T; |
| | | Z₃ is selected from the group consisting of E and T; |
| | | Z₄ is selected from the group consisting of A and K; and |
| | | X is selected from any amino acid. |
| 20 | N-terminal | DLGXXLLXAAXXGQLDXVRXLLXAGADVNA |
| | capping module | wherein X is selected from any amino acid. |
| 21 | N-terminal | DLGXXLLXAAXXGQXDXVRXLLXAGADVNA |
| | capping module | wherein X is selected from any amino acid. |
| 22 | N-terminal | DLGXKLLXAAXXGQDDEVRILLAAGADVNA |
| | capping module | wherein X is selected from any amino acid. |
| 23 | N-terminal | DLGXKLLXAAXXGQLDEVRILLAAGADVNA |
| | capping module | wherein X is selected from any amino acid. |
| 24 | N-terminal | DLGXKLLXAAXXGQDDEVRELLKAGADVNA |
| | capping module | wherein X is selected from any amino acid. |
| 25 | N-terminal | DLGXKLLXAAXXGQLDEVRELLKAGADVNA |
| | capping module | wherein X is selected from any amino acid. |
| 26 | Ankyrin repeat | SDNDGYTPLHLAASEGHLEIVEVLLEAGADVNA |
| | module | |
| 27 | Ankyrin repeat | KDNDGYTPLHLAASEGHLEIVEVLLEAGADVNA |
| | module | |
| 28 | Ankyrin repeat | KDNSGYTPLHLAASSGNLEIVEVLLSAGADVNA |
| | module | |
| 29 | Ankyrin repeat | QDEYGLTPLYLATAHGHLQIVQVLLSAGADVNA |
| | module | |
| 30 | Ankyrin repeat | QDAIGFTPLHLAAFIGHLQIAQVLLSAGADVNA |
| | module | |
| 31 | Ankyrin repeat | Z₁DXXGXTPLZ₂XAZ₃XXGZ₄LZ₅IZ₆Z-VLLZ&AGADVNA |
| | module | wherein Z₁ is selected from the group consisting of S, K, and Q; |
| | | Z₂ is selected from the group consisting of H and Y; |
| | | Z₃ is selected from the group consisting of A and T; |
| | | Z₄ is selected from the group consisting of H and N; |
| | | Z₅ is selected from the group consisting of Q and E; |
| | | Z₆ is selected from the group consisting of V and A; |
| | | Z₇ is selected from the group consisting of Q and E; |
| | | Z₈ is selected from the group consisting of Q and K; and |
| | | X is selected from any amino acid. |
| 32 | Ankyrin repeat | XDXXGXTPLXXAXXXGXLXIXXVLLXAGADVNA |
| | module | wherein X is selected from any amino acid. |
| 33 | Ankyrin repeat | XDXXGXTPLHLAAXXGHLEIVEVLLKXGADVNA |
| | module | wherein X is selected from any amino acid. |
| 34 | Ankyrin repeat | KDXXGXTPLHLAAXXGHLEIVEVLLKAGADVNA |
| | module | wherein X is selected from any amino acid. |
| 35 | Ankyrin repeat | KDXXGXTPLHXAAXXGHLEIVEVLLKAGADVNA |
| | module | wherein X is selected from any amino acid. |
| 36 | C-terminal | QDTSGSTPADLAADAGNEDIAEVLQKAA |
| | capping module | |
| 37 | C-terminal | QDTSGSTPADLAADAGHEDIAEVLQQAA |
| | capping module | |
| 38 | C-terminal | QDTSGSTPADLAATAGHEDIAEVLQKAA |
| | capping module | |
| 39 | C-terminal | QDKFGTTPAQIAATAGNQQIASVLQQAA |
| | capping module | |
| 40 | C-terminal | QDXXGXTPAZIZ₂AAXXGZ₃Z₄Z₅IAZ&VLQZ-AA |
| | capping module | wherein Z₁ is selected from the group consisting of Q and D; |
| | | Z₂ is selected from the group consisting of I and L; |
| | | Z₃ is selected from the group consisting of H and N; |
| | | Z₄ is selected from the group consisting of E and Q; |
| | | Z₅ is selected from the group consisting of D and Q; |
| | | Z₆ is selected from the group consisting of E and S; |
| | | Z₇ is selected from the group consisting of K and Q; and |
| | | X is selected from any amino acid. |
| 41 | C-terminal | QDXXGXTPAXXAAXXGXXXIAXVLQXAA |
| | capping module | wherein X is selected from any amino acid. |
| 42 | C-terminal | QDXXGXTPADLAAXXGHEDIAEVLQKLN |
| | capping module | wherein X is selected from any amino acid. |
| 43 | N-terminal | DLGTQLLRAAESGQLDEVRELLKAGADVNA |
| | capping module | |
| 44 | N-terminal | DLGTQLLQAAESGQLDTVRELLKAGADVNA |
| | capping module | |
| 45 | N-terminal | DLGIQLLFAAAKGQLDTVRTLLQAGADVNA |
| | capping module | |
| 46 | Ankyrin repeat | KDKDGYTPLHLAAREGHLEIVEVLLSAGADVNA |
| | module | |
| 47 | Ankyrin repeat | QDEYGLTPLYLATAHGHLEIVTVLLEAGADVNA |
| | module | |
| 48 | Ankyrin repeat | QDAIGFTPLHLAAFIGHLEIATVLLEAGADVNA |
| | module | |
| 49 | Ankyrin repeat | QDEYGLTPLYLATAHGHLQIVRVLLQAGADVNA |
| | module | |
| 50 | Ankyrin repeat | QDAIGFTPLHLAAFIGHLEIATVLLKAGADVNA |
| | module | |
| 51 | Ankyrin repeat | KDFQGVTPLHIAAQSGHLQIVEVLLQAGADVNA |
| | module | |
| 52 | Ankyrin repeat | KDVTGDTPLHLAAQHGHLQIVTVLLQAGADVNA |
| | module | |
| 53 | C-terminal | QDTSGSTPADLAADAGHEDIAEVLQKAA |
| | capping module | |
| 54 | C-terminal | QDKFGKTPADIAAGAGNEDIAEVLQKAA |
| | capping module | |
| 55 | C-terminal | QDKFGKTPADIAATAGNQQIASVLQKAA |
| | capping module | |
| 56 | C-terminal | QDERGWTPADLAADWGNQQIAEVLQQAA |
| | capping module | |
| 57 | N-terminal | DXXXXXXXXXXXXXXXXRXXXXXXXDXXX |
| | capping module | wherein X is selected from any amino acid. |
| 58 | N-terminal | Z₁XXXXXXXXXXXXXXZ₂XXZ₃XXXXXXXZ₄XXX |
| | capping module | wherein Z₁ is selected from the group consisting of D and E; |
| | | Z₂ is selected from the group consisting of D and E; |
| | | Z₃ is selected from the group consisting of R, K and H; |
| | | Z₄ is selected from the group consisting of D and E; and |
| | | X is selected from any amino acid. |
| 59 | N-terminal | DXXXXXXXXXXXXXZDXXRXXXXXXXDXXX |
| | capping module | wherein Z is selected from the group consisting of L, V, I, A, M and T; and |
| | | X is selected from any amino acid. |
| 60 | N-terminal | Z₁XXXXXXXXXXXXXZ₂Z₃XXZ₄XXXXXXXZ₅XXX |
| | capping module | wherein Z₁ is selected from the group consisting of D and E; |
| | | Z₂ is selected from the group consisting of L, V, I, A, M and T; |
| | | Z₃ is selected from the group consisting of D and E; |
| | | Z₄ is selected from the group consisting of R, K and H; |
| | | Z₅ is selected from the group consisting of D and E; and |
| | | X is selected from any amino acid. |
| 61 | Ankyrin repeat | XDXXXXXXXXXXXXXXXXXXXXXXXXXXXDXXX |
| | module | wherein X is selected from any amino acid. |
| 62 | Ankyrin repeat | XZ₁XXXXXXXXXXXXXXXXXXXXXXXXXXXZ₂XXX |
| | module | wherein Z₁ is selected from the group consisting of D and E; |
| | | Z₂ is selected from the group consisting of D and E; and |
| | | X is selected from any amino acid. |
| 63 | C-terminal | XDXXXXXXXXXXXXXXXXXXXXXXXXXX |
| | capping module | wherein X is selected from any amino acid. |
| 64 | C-terminal | XZ₁XXXXXXXXXXXXXXXXXXXXXXXXXX |
| | capping module | wherein Z₁ is selected from the group consisting of D and E; and |
| | | X is selected from any amino acid. |
| 65 | N-terminal | DLGXKLLEAAXXGQDDEVRELLKAGADVNA |
| | capping module | wherein X is selected from any amino acid. |
| 66 | N-terminal | DLGXKLLEAAXXGQDDXVRXLLXAGADVNA |
| | capping module | wherein X is selected from any amino acid. |
| 67 | N-terminal | DLGXXLLEAAXXGQDDXVRXLXXXGADVNA |
| | capping module | wherein X is selected from any amino acid. |
| 68 | N-terminal | XXXXXLLEAAXXGXDXXXXXXXXXGADVNA |
| | capping module | wherein X is selected from any amino acid. |
| 69 | N-terminal | XXXXXXXEXXXXXXDXXXXXXXXXGADVNA |
| | capping module | wherein X is selected from any amino acid. |
| 70 | N-terminal | XXXXXXXEXXXXXXXXXXXXXXXXGADVNA |
| | capping module | wherein Z is selected from the group consisting of L, V, I, A, M and T; and |
| | | X is selected from any amino acid. |
| 71 | N-terminal | DLGXKLLQAAXXGQLDEVRELLKAGADVNA |
| | capping module | wherein X is selected from any amino acid. |
| 72 | N-terminal | DLGXKLLQAAXXGQLDXVRXLLXAGADVNA |
| | capping module | wherein X is selected from any amino acid. |
| 73 | N-terminal | DLGXXLLQAAXXGQLDXVRXLXXXGADVNA |
| | capping module | wherein X is selected from any amino acid. |
| 74 | N-terminal | XXXXXLLQAAXXGXLXXXXXXXXXGADVNA |
| | capping module | wherein X is selected from any amino acid. |
| 75 | N-terminal | XXXXXXXQXXXXXXLXXXXXXXXXGADVNA |
| | capping module | wherein X is selected from any amino acid. |
| 76 | N-terminal | XXXXXXXQXXXXXXXXXXXXXXXXGADVNA |
| | capping module | wherein Z is selected from the group consisting of L, V, I, A, M and T; and |
| | | X is selected from any amino acid. |
| 77 | N-terminal | DLGXKLLQAAXXGQLDEVRELLKA |
| | capping module | wherein X is selected from any amino acid. |
| 78 | N-terminal | DLGXKLLQAAXXGQLDXVRXLLXA |
| | capping module | wherein X is selected from any amino acid. |
| 79 | N-terminal | DLGXXLLQAAXXGQLDXVRXLXXX |
| | capping module | wherein X is selected from any amino acid. |
| 80 | N-terminal | DXXXXXXXXXXXXXZDXXRXXXXX |
| | capping module | wherein Z is selected from the group consisting of L, V, I, A, M and T; and |
| | | X is selected from any amino acid. |
| 81 | N-terminal | Z₁XXXXXXXXXXXXXZ₂Z₃XXZ₄XXXXX |
| | capping module | wherein Z₁ is selected from the group consisting of D and E; |
| | | Z₂ is selected from the group consisting of L, V, I, A, M and T; |
| | | Z₃ is selected from the group consisting of D and E; |
| | | Z₄ is selected from the group consisting of R, K and H; and |
| | | X is selected from any amino acid. |
| 82 | C-terminal | XDXXGXTPADXAADXGHEDIAEVLQKAA |
| | capping module | wherein X is selected from any amino acid. |
| 83 | C-terminal | XDXXGXTPADXAADXGHEXIAXVLQXAA |
| | capping module | wherein X is selected from any amino acid. |
| 84 | C-terminal | XDXXGXTPXXXAADXGXEXXXXXXXXAA |
| | capping module | wherein X is selected from any amino acid. |
| 85 | C-terminal | XDXXGXTPXXXXADXXXEXXXXXXXXXX |
| | capping module | wherein X is selected from any amino acid. |
| 86 | C-terminal | XDXXGXTPXXXXXDXXXEXXXXXXXXAA |
| | capping module | wherein X is selected from any amino acid. |
| 87 | C-terminal | XDXXGXTPADXAARXGHQDIAEVLQKAA |
| | capping module | wherein X is selected from any amino acid. |
| 88 | C-terminal | XDXXGXTPADXAARXGHQXIAXVLQXAA |
| | capping module | wherein X is selected from any amino acid. |
| 89 | C-terminal | XDXXGXTPXXXAARXGXQXXXXXXXXAA |
| | capping module | wherein X is selected from any amino acid. |
| 90 | C-terminal | XDXXGXTPXXXXARXXXQXXXXXXXXXX |
| | capping module | wherein X is selected from any amino acid. |
| 91 | C-terminal | XDXXGXTPXXXXXXXXQXXXXXXXXAA |
| | capping module | wherein X is selected from any amino acid. |
| 92 | Ankyrin repeat | XDXXGXTPLHXAAXXGHLEIVEVLLKXGADVNA |
| | module | wherein X is selected from any amino acid. |
| 93 | Ankyrin repeat | XDXXGXTPLHXAAXXGHPEIVEVLLKXGADVNA |
| | module | wherein X is selected from any amino acid. |
| 94 | Ankyrin repeat | XDXXGXTPLHXAXXXGXXXIVXVLLXXGADVNA |
| | module | wherein X is selected from any amino acid. |
| 95 | Designed ankyrin | DLGTQLLQAAESGQLDEVHDLLQAGADVNAQDEYGLTPLYLATAHGHLEIVTVLLEAG [symbol] |
| | repeat domain | HLAAFIGHLEIATVLLEAGADVNAQDKFGKTPADIAAGAGNEDIAEVLQKAA |
| 96 | Designed ankyrin | DLGTQLLEAAESGQLDEVRELLQAGADVNAQDEYGLTPLYLATAHGHLEIVTVLLEAG [symbol] |
| | repeat domain | HLAAFIGHLEIATVLLEAGADVNAQDKFGKTPADIAAGAGNEDIAEVLQKAA |
| 97 | Designed ankyrin | DLGTQLLRAAESGQLDEVRELLKAGADVNAQDEYGLTPLYLATAHGHLEIVTVLLEAG [symbol] |
| | repeat domain | HLAAFIGHLEIATVLLEAGADVNAQDNFGTTPADIAAGAGNEDIAEVLQQAA |
| 98 | Designed ankyrin | DLGTQLLEAAESGQLDEVRELLKAGADVNAQDEYGLTPLYLATAHGHLEIVTVLLEAG [symbol] |
| | repeat domain | HLAAFIGHLEIATVLLEAGADVNAQDKFGTTPADIAAGAGNEDIAEVLQKAA |
| 99 | N-terminal | DLGTQLLQAAESGQLDEVHDLLQAGADVNA |
| | capping module | |
| 100 | N-terminal | DLGTQLLEAAESGQLDEVRELLQAGADVNA |
| | capping module | |
| 101 | N-terminal | DLGTQLLEAAESGQLDEVRELLKAGADVNA |
| | capping module | |
| 102 | C-terminal | QDNFGTTPADIAAGAGNEDIAEVLQQAA |
| | capping module | |
| 103 | C-terminal | QDKFGTTPADIAAGAGNEDIAEVLQKAA |
| | capping module | |
| 104 | Designed ankyrin | DLGXQLLXAAXXGQLDTVRELLAAGADVNASDXXGXTPLHXAAXXGHLEIVEVLLEAG [symbol] |
| | repeat domain | LHXAAXXGHLEIVEVLLEAGADVNAQDXXGXTPADLAAXXGNEDIAEVLQKAA |
| | | wherein X is selected from any amino acid. |
| 105 | Designed ankyrin | DLGXKLLXAAXXGQLDEVRELLKAGADVNAKDXXGXTPLHXAAXXGHLEIVEVLLEAG [symbol] |
| | repeat domain | HXAAXXGHLEIVEVLLEAGADVNAQDXXGXTPADLAAXXGHEDIAEVLQQAA |
| | | wherein X is selected from any amino acid. |
| 106 | Designed ankyrin | DLGXKLLXAAXXGQLDEVRELLKAGADVNAKDXXGXTPLHXAAXXGHLEIVEVLLSAG [symbol] |
| | repeat domain | HXAAXXGHLEIVEVLLSAGADVNAQDXXGXTPADLAAXXGHEDIAEVLQKAA |
| | | wherein X is selected from any amino acid. |
| 107 | Designed ankyrin | DLGXQLLXAAXXGQLDEVRELLAAGADVNAKDXXGXTPLHXAAXXGNLEIVEVLLSAG [symbol] |
| | repeat domain | LHXAAXXGNLEIVEVLLSAGADVNAQDXXGXTPADLAAXXGHEDIAEVLQKAA |
| | | wherein X is selected from any amino acid. |
| 108 | Designed ankyrin | DLGXQLLXAAXXGOLDEVRELLKAGADVNAQDXXGXTPLYXATXXGHLEIVTVLLEAG [symbol] |
| | repeat domain | LHXAAXXGHLEIATVLLEAGADVNAQDXXGXTPADIAAXXGNEDIAEVLQKAA |
| | | wherein X is selected from any amino acid. |
| 109 | Designed ankyrin | DLGXQLLXAAXXGQLDTVRTLLKAGADVNAQDXXGXTPLYXATXXGHLQIVQVLLAG [symbol] |
| | repeat domain | LHXAAXXGHLQIAQVLLSAGADVNAQDXXGXTPAQIAAXXGNQQIASVLQQAA |
| | | wherein X is selected from any amino acid. |
| 110 | Designed ankyrin | DLGXQLLXAAXXGQLDTVRELLKAGADVNAQDXXGXTPLYXATXXGHLQIVRVLLQAG [symbol] |
| | repeat domain | LHXAAXXGHLEIATVLLKAGADVNAQDXXGXTPADIAAXXGNQQIASVLQKAA |
| | | wherein X is selected from any amino acid. |
| 111 | Designed ankyrin | DLGXQLLXAAXXGQLDTVRTLLQAGADVNAKDXXGXTPLHXAAXXGHLQIVEVLLQAG [symbol] |
| | repeat domain | LHXAAXXGHLQIVTVLLQAGADVNAQDXXGXTPADLAAXXGNQQIAEVLQQAA |
| | | wherein X is selected from any amino acid. |
| 112 | Designed ankyrin | DLGXQLLXAAXXGQLDEVHDLLQAGADVNAQDXXGXTPLYXATXXGHLEIVTVLLEAG [symbol] |
| | repeat domain | LHXAAXXGHLEIATVLLEAGADVNAQDXXGXTPADIAAXXGNEDIAEVLQKAA |
| | | wherein X is selected from any amino acid. |
| 113 | Designed ankyrin | DLGXQLLXAAXXGQLDEVRELLQAGADVNAQDXXGXTPLYXATXXGHLEIVTVLLEAG [symbol] |
| | repeat domain | LHXAAXXGHLEIATVLLEAGADVNAQDXXGXTPADIAAXXGNEDIAEVLQKAA |
| | | wherein X is selected from any amino acid. |
| 114 | Designed ankyrin | DLGXQLLXAAXXGQLDEVRELLKAGADVNAQDXXGXTPLYXATXXGHLEIVTVLLEAG [symbol] |
| | repeat domain | LHXAAXXGHLEIATVLLEAGADVNAQDXXGXTPADIAAXXGNEDIAEVLQQAA |
| | | wherein X is selected from any amino acid. |
| 115 | N-terminal | DLGXQLLXAAXXGQLDTVRELLAAGADVNA |
| | capping module | wherein X is selected from any amino acid. |
| 116 | N-terminal | DLGXQLLXAAXXGQLDEVRELLAAGADVNA |
| | capping module | wherein X is selected from any amino acid. |
| 117 | N-terminal | DLGXQLLXAAXXGQLDEVRELLKAGADVNA |
| | capping module | wherein X is selected from any amino acid. |
| 118 | N-terminal | DLGXQLLXAAXXGQLDTVRTLLKAGADVNA |
| | capping module | wherein X is selected from any amino acid. |
| 119 | N-terminal | DLGXQLLXAAXXGQLDTVRELLKAGADVNA |
| | capping module | wherein X is selected from any amino acid. |
| 120 | N-terminal | DLGXQLLXAAXXGQLDTVRTLLQAGADVNA |
| | capping module | wherein X is selected from any amino acid. |
| 121 | N-terminal | DLGXQLLXAAXXGQLDEVHDLLQAGADVNA |
| | capping module | wherein X is selected from any amino acid. |
| 122 | N-terminal | DLGXQLLXAAXXGQLDEVRELLQAGADVNA |
| | capping module | wherein X is selected from any amino acid. |
| 123 | C-terminal | QDXXGXTPADLAAXXGNEDIAEVLQKAA |
| | capping module | wherein X is selected from any amino acid. |
| 124 | C-terminal | QDXXGXTPADLAAXXGHEDIAEVLQQAA |
| | capping module | wherein X is selected from any amino acid. |
| 125 | C-terminal | QDXXGXTPADLAAXXGHEDIAEVLQKAA |
| | capping module | wherein X is selected from any amino acid. |
| 126 | C-terminal | QDXXGXTPADIAAXXGNEDIAEVLQKAA |
| | capping module | wherein X is selected from any amino acid. |
| 127 | C-terminal | QDXXGXTPAQIAAXXGNQQIASVLQQAA |
| | capping module | wherein X is selected from any amino acid. |
| 128 | C-terminal | QDXXGXTPADIAAXXGNQQIASVLQKAA |
| | capping module | wherein X is selected from any amino acid. |
| 129 | C-terminal | QDXXGXTPADLAAXXGNQQIAEVLQQAA |
| | capping module | wherein X is selected from any amino acid. |
| 130 | C-terminal | QDXXGXTPADIAAXXGNEDIAEVLQQAA |
| | capping module | wherein X is selected from any amino acid. |
| 131 | Ankyrin repeat | SDXXGXTPLHXAAXXGHLEIVEVLLEAGADVNA |
| | module | wherein X is selected from any amino acid. |
| 132 | Ankyrin repeat | KDXXGXTPLHXAAXXGHLEIVEVLLEAGADVNA |
| | module | wherein X is selected from any amino acid. |
| 133 | Ankyrin repeat | KDXXGXTPLHXAAXXGHLEIVEVLLSAGADVNA |
| | module | wherein X is selected from any amino acid. |
| 134 | Ankyrin repeat | KDXXGXTPLHXAAXXGNLEIVEVLLSAGADVNA |
| | module | wherein X is selected from any amino acid. |
| 135 | Ankyrin repeat | QDXXGXTPLYXATXXGHLEIVTVLLEAGADVNA |
| | module | wherein X is selected from any amino acid. |
| 136 | Ankyrin repeat | QDXXGXTPLYXATXXGHLQIVQVLLSAGADVNA |
| | module | wherein X is selected from any amino acid. |
| 137 | Ankyrin repeat | QDXXGXTPLYXATXXGHLQIVRVLLQAGADVNA |
| | module | wherein X is selected from any amino acid. |
| 138 | Ankyrin repeat | KDXXGXTPLHXAAXXGHLQIVEVLLQAGADVNA |
| | module | wherein X is selected from any amino acid. |
| 139 | Ankyrin repeat | QDXXGXTPLHXAAXXGHLEIATVLLEAGADVNA |
| | module | wherein X is selected from any amino acid. |
| 140 | Ankyrin repeat | QDXXGXTPLHXAAXXGHLQIAQVLLSAGADVNA |
| | module | wherein X is selected from any amino acid. |
| 141 | Ankyrin repeat | QDXXGXTPLHXAAXXGHLEIATVLLKAGADVNA |
| | module | wherein X is selected from any amino acid. |
| 142 | Ankyrin repeat | KDXXGXTPLHXAAXXGHLQIVTVLLQAGADVNA |
| | module | wherein X is selected from any amino acid. |

[symbol]

indicates data missing or illegible when filed

1-65. (canceled)

66. A designed ankyrin repeat domain comprising an N-terminal capping module, at least one internal repeat module and a C-terminal capping module, wherein said repeat domain has at least one characteristic selected from the following list of characteristics:
(a) an isoelectric point (pI) equal to or lower than pH 4.07;
(b) among all amino acid residues comprised in said repeat domain, a percentage of basic amino acid residues equal to or lower than 7.0%; and
(c) among the framework residues comprised in said repeat domain, a percentage of basic amino acid residues equal to or lower than 6.1%.

67. The repeat domain according to claim 66, wherein said repeat domain binds specifically to a target, optionally wherein said repeat domain binds said target with a dissociation constant (K_{D}) lower than 10⁻⁷ M.

68. The repeat domain according to claim 66, wherein said repeat domain comprises one internal repeat module, two internal repeat modules, three internal repeat modules, or four internal repeat modules.

69. The repeat domain according to claim 66, wherein each internal repeat module independently comprises a sequence selected from the group consisting of (1) SEQ ID NOs: 131 to 142 and (2) sequences in which up to 9 framework residues in any of SEQ ID NOs: 131 to 142 are substituted by another amino acid, and/or said N-terminal capping module comprises a sequence selected from the group consisting of (1) SEQ ID NOs: 25 and 115 to 122 and (2) sequences in which up to 9 framework residues in any of SEQ ID NOs: 25 and 115 to 122 are substituted by another amino acid, and/or said C-terminal capping module comprises a sequence selected from the group consisting of (1) SEQ ID NOs: 123 to 130 and (2) sequences in which up to 9 framework residues in any of SEQ ID NOs: 123 to 130 are substituted by another amino acid.

70. The repeat domain according to claim 66, wherein the repeat domain comprises an amino acid sequence selected from the group consisting of (1) SEQ ID NOs: 104, 108, 109 and 112 to 114 and (2) sequences with at least 80% amino acid sequence identity among the framework residues of any one of SEQ ID NOs: 104, 108, 109 and 112 to 114.

71. The repeat domain according to claim 66, wherein the repeat domain comprises an amino acid sequence selected from the group consisting of (1) SEQ ID NOs: 8 to 9 and 95 to 98 and (2) sequences with at least 80% amino acid sequence identity with any one of SEQ ID NOs: 8 to 9 and 95 to 98.

72. A recombinant protein comprising the repeat domain according to claim 66.

73. A nucleic acid encoding the repeat domain according to claim 66.

74. A method of generating a modified ankyrin repeat domain, the method comprising steps (a) and (b),
wherein step (a) is providing an ankyrin repeat domain, which has an isoelectric point (pI) higher than pH 4.07, and/or among all amino acid residues comprised in the repeat domain, a percentage of basic amino acid residues higher than 7.0%, and/or among the framework residues comprised in the repeat domain, a percentage of basic amino acid residues higher than 6.1%; and
wherein step (b) is (1) substituting at least one basic amino acid residue of the repeat domain of step (a) with a neutral or acidic amino acid residue; and/or
(2) substituting at least one neutral amino acid residue of the repeat domain of step (a) with an acidic amino acid;
wherein said modified ankyrin repeat domain has at least one characteristic selected from the following list of characteristics:
(i) an isoelectric point (pI) equal to or lower than pH 4.07;
(ii) among all amino acid residues comprised in the repeat domain, a percentage of basic amino acid residues equal to or lower than 7.0%; and
(iii) among the framework residues comprised in the repeat domain, a percentage of basic amino acid residues equal to or lower than 6.1%.

75. The method according to claim 74, wherein the repeat domain of step (a) binds specifically to a target, and wherein said modified repeat domain binds specifically said target with a K_{D} lower than 10⁻⁷ M.

76. The method according to claim 74, wherein said modified repeat domain comprises one internal repeat module, two internal repeat modules, three internal repeat modules, or four internal repeat modules.

77. A designed ankyrin repeat domain comprising an N-terminal capping module, at least one internal repeat module and a C-terminal capping module, wherein said repeat domain is linked to a drug moiety, and wherein said repeat domain has at least one characteristic selected from the following list of characteristics:
(a) an isoelectric point (pI) equal to or lower than pH 4.6;
(b) among all amino acid residues comprised in said repeat domain, a percentage of basic amino acid residues equal to or lower than 12.0%;
(c) among all amino acid residues comprised in said repeat domain, a percentage of Arg and Lys residues equal to or lower than 8.0%;
(d) among the framework residues comprised in said repeat domain, a percentage of basic amino acid residues equal to or lower than 9.6%; and
(e) among the framework residues comprised in said repeat domain, a percentage of Arg and Lys residues equal to or lower than 6.7%.

78. The repeat domain according to claim 77, wherein said repeat domain comprises one internal repeat module, two internal repeat modules, three internal repeat modules, or four internal repeat modules.

79. The repeat domain according to claim 77, wherein said repeat domain binds specifically to a target, optionally wherein said repeat domain binds said target with a dissociation constant (K_{D}) lower than 10⁻⁷ M.

80. The repeat domain according to claim 77, wherein said drug moiety is a toxin.

81. The repeat domain according to claim 78, wherein said toxin is a radionuclide or a cytotoxin.

82. The repeat domain according to claim 77, wherein said repeat domain comprises an amino acid sequence selected from the group consisting of (1) SEQ ID NOs: 104 to 114 and (2) sequences with at least 80% amino acid sequence identity among the framework residues of any one of SEQ ID NOs: 104 to 114.

83. The repeat domain according to claim 77, wherein said repeat domain comprises an amino acid sequence selected from the group consisting of (1) SEQ ID NOs: 8 to 10, 12 and 95 to 98 and (2) sequences with at least 80% amino acid sequence identity with any one of SEQ ID NOs: 8 to 10, 12 and 95 to 98.

84. A recombinant protein comprising the repeat domain according to claim 77.

85. A nucleic acid encoding the repeat domain according to claim 77.

86. A pharmaceutical composition comprising the recombinant protein according to claim 85, and optionally a pharmaceutically acceptable carrier or diluent.

87. A method of treating and/or diagnosing a medical condition, the method comprising the step of administering to a patient in need thereof a therapeutically and/or diagnostically effective amount of the recombinant protein according to claim 85.

88. The method of claim 87, wherein said medical condition is a cancer.


SEQUENCE LISTING (142 sequences)

SEQ ID NO: 1 - protein, 124 aa, synthetic construct
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 2 - protein, 124 aa, synthetic construct
DLGTQLLEAAQAGQLDTVRELLAAGADVNASDNDGYTPLHLAASEGHLEIVEVLLEAGADVNASDNDGYTPLHLAASEGHLEIVEVLLEAGADVNAQDTSGSTPADLAADAGNEDIAEVLQKAA

SEQ ID NO: 3 - protein, 124 aa, synthetic construct
DLGSKLLEAAQAGQLDEVRELLKAGADVNAKDNDGYTPLHLAASEGHLEIVEVLLEAGADVNAKDNDGYTPLHLAASEGHLEIVEVLLEAGADVNAQDTSGSTPADLAADAGHEDIAEVLQQAA

SEQ ID NO: 4 - protein, 124 aa, synthetic construct
DLGSKLLEAAQAGQLDEVRELLKAGADVNAKDKDGYTPLHLAAREGHLEIVEVLLSAGADVNAKDKDGYTPLHLAAREGHLEIVEVLLSAGADVNAQDTSGSTPADLAADAGHEDIAEVLQKAA

SEQ ID NO: 5 - protein, 124 aa, synthetic construct
DLGTQLLQAAQAGQLDEVRELLAAGADVNAKDNSGYTPLHLAASSGNLEIVEVLLSAGADVNAKDNSGYTPLHLAASSGNLEIVEVLLSAGADVNAQDTSGSTPADLAATAGHEDIAEVLQKAA

SEQ ID NO: 6 - protein, 124 aa, synthetic construct
DLGKKLLEAARAGQDDEVRELLKAGADVNAKDEYGLTPLYLATAHGHLEIVEVLLKAGADVNAVDAIGFTPLHLAAFIGHLEIAEVLLKAGADVNAQDKFGKTPADIAAGAGNEDIAEVLQKAA

SEQ ID NO: 7 - protein, 124 aa, synthetic construct
DLGTQLLRAAESGQLDTVRTLLKAGADVNAKDEYGLTPLYLATAHGHLQIVRVLLQAGADVNAKDAIGFTPLHLAAFIGHLKIAEVLLKAGADVNAQDKFGKTPAQIAATAGNQQIASVLQKAA

SEQ ID NO: 8 - protein, 124 aa, synthetic construct
DLGTQLLRAAESGQLDEVRELLKAGADVNAQDEYGLTPLYLATAHGHLEIVTVLLEAGADVNAQDAIGFTPLHLAAFIGHLEIATVLLEAGADVNAQDKFGKTPADIAAGAGNEDIAEVLQKAA

SEQ ID NO: 9 - protein, 124 aa, synthetic construct
DLGTQLLQAAESGQLDTVRTLLKAGADVNAQDEYGLTPLYLATAHGHLQIVQVLLSAGADVNAQDAIGFTPLHLAAFIGHLQIAQVLLSAGADVNAQDKFGTTPAQIAATAGNQQIASVLQQAA

SEQ ID NO: 10 - protein, 124 aa, synthetic construct
DLGTQLLQAAESGQLDTVRELLKAGADVNAQDEYGLTPLYLATAHGHLQIVRVLLQAGADVNAQDAIGFTPLHLAAFIGHLEIATVLLKAGADVNAQDKFGKTPADIAATAGNQQIASVLQKAA

SEQ ID NO: 11 - protein, 124 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAA

SEQ ID NO: 12 - protein, 124 aa, synthetic construct
DLGIQLLFAAAKGQLDTVRTLLQAGADVNAKDFQGVTPLHIAAQSGHLQIVEVLLQAGADVNAKDVTGDTPLHLAAQHGHLQIVTVLLQAGADVNAQDERGWTPADLAADWGNQQIAEVLQQAA

SEQ ID NO: 13 - protein, 16 aa, synthetic construct
MRGSHHHHHHENLYFQ

SEQ ID NO: 14 - protein, 5 aa, synthetic construct
GSGSC

SEQ ID NO: 15 - protein, 30 aa, synthetic construct
DLGTQLLEAAQAGQLDTVRELLAAGADVNA

SEQ ID NO: 16 - protein, 30 aa, synthetic construct
DLGSKLLEAAQAGQLDEVRELLKAGADVNA

SEQ ID NO: 17 - protein, 30 aa, synthetic construct
DLGTQLLQAAQAGQLDEVRELLAAGADVNA

SEQ ID NO: 18 - protein, 30 aa, synthetic construct
DLGTQLLQAAESGQLDTVRTLLKAGADVNA

SEQ ID NO: 19 - protein, 30 aa, synthetic construct
  VARIANT 4: note = X is selected from any amino acid
  VARIANT 5: note = X is selected from the group consisting of Q and K
  VARIANT 8..12: note = X is selected from any amino acid
  VARIANT 17: note = X is selected from the group consisting of E and T
  VARIANT 20: note = X is selected from the group consisting of E and T
  VARIANT 23: note = X is selected from the group consisting of A and K
DLGXXLLXAAXXGQLDXVRXLLXAGADVNA

SEQ ID NO: 20 - protein, 30 aa, synthetic construct; identical to SEQ ID NO: 19
  VARIANT 4..23: note = X is selected from any amino acid

SEQ ID NO: 21 - protein, 30 aa, synthetic construct
  VARIANT 4..23: note = X is selected from any amino acid
DLGXXLLXAAXXGQXDXVRXLLXAGADVNA

SEQ ID NO: 22 - protein, 30 aa, synthetic construct
  VARIANT 4..12: note = X is selected from any amino acid
DLGXKLLXAAXXGQDDEVRILLAAGADVNA

SEQ ID NO: 23 - protein, 30 aa, synthetic construct
  VARIANT 4..12: note = X is selected from any amino acid
DLGXKLLXAAXXGQLDEVRILLAAGADVNA

SEQ ID NO: 24 - protein, 30 aa, synthetic construct
  VARIANT 4..12: note = X is selected from any amino acid
DLGXKLLXAAXXGQDDEVRELLKAGADVNA

SEQ ID NO: 25 - protein, 30 aa, synthetic construct
  VARIANT 4..12: note = X is selected from any amino acid
DLGXKLLXAAXXGQLDEVRELLKAGADVNA

SEQ ID NO: 26 - protein, 33 aa, synthetic construct
SDNDGYTPLHLAASEGHLEIVEVLLEAGADVNA

SEQ ID NO: 27 - protein, 33 aa, synthetic construct
KDNDGYTPLHLAASEGHLEIVEVLLEAGADVNA

SEQ ID NO: 28 - protein, 33 aa, synthetic construct
KDNSGYTPLHLAASSGNLEIVEVLLSAGADVNA

SEQ ID NO: 29 - protein, 33 aa, synthetic construct
QDEYGLTPLYLATAHGHLQIVQVLLSAGADVNA

SEQ ID NO: 30 - protein, 33 aa, synthetic construct
QDAIGFTPLHLAAFIGHLQIAQVLLSAGADVNA

SEQ ID NO: 31 - protein, 33 aa, synthetic construct
  VARIANT 1: note = X is selected from the group consisting of S, K and Q
  VARIANT 3..6: note = X is selected from any amino acid
  VARIANT 10: note = X is selected from the group consisting of H and Y
  VARIANT 11: note = X is selected from any amino acid
  VARIANT 13: note = X is selected from the group consisting of A and T
  VARIANT 14..15: note = X is selected from any amino acid
  VARIANT 17: note = X is selected from the group consisting of H and N
  VARIANT 19: note = X is selected from the group consisting of Q and E
  VARIANT 21: note = X is selected from the group consisting of V and A
  VARIANT 22: note = X is selected from the group consisting of Q and E
  VARIANT 26: note = X is selected from the group consisting of Q and K
XDXXGXTPLXXAXXXGXLXIXXVLLXAGADVNA

SEQ ID NO: 32 - protein, 33 aa, synthetic construct; identical to SEQ ID NO: 31
  VARIANT 1..26: note = X is selected from any amino acid

SEQ ID NO: 33 - protein, 33 aa, synthetic construct
  VARIANT 1..27: note = X is selected from any amino acid
XDXXGXTPLHLAAXXGHLEIVEVLLKXGADVNA

SEQ ID NO: 34 - protein, 33 aa, synthetic construct
  VARIANT 3..15: note = X is selected from any amino acid
KDXXGXTPLHLAAXXGHLEIVEVLLKAGADVNA

SEQ ID NO: 35 - protein, 33 aa, synthetic construct
  VARIANT 3..15: note = X is selected from any amino acid
KDXXGXTPLHXAAXXGHLEIVEVLLKAGADVNA

SEQ ID NO: 36 - protein, 28 aa, synthetic construct
QDTSGSTPADLAADAGNEDIAEVLQKAA

SEQ ID NO: 37 - protein, 28 aa, synthetic construct
QDTSGSTPADLAADAGHEDIAEVLQQAA

SEQ ID NO: 38 - protein, 28 aa, synthetic construct
QDTSGSTPADLAATAGHEDIAEVLQKAA

SEQ ID NO: 39 - protein, 28 aa, synthetic construct
QDKFGTTPAQIAATAGNQQIASVLQQAA

SEQ ID NO: 40 - protein, 28 aa, synthetic construct
  VARIANT 3..6: note = X is selected from any amino acid
  VARIANT 10: note = X is selected from the group consisting of Q and D
  VARIANT 11: note = X is selected from the group consisting of I and L
  VARIANT 14..15: note = X is selected from any amino acid
  VARIANT 17: note = X is selected from the group consisting of H and N
  VARIANT 18: note = X is selected from the group consisting of E and Q
  VARIANT 19: note = X is selected from the group consisting of D and Q
  VARIANT 22: note = X is selected from the group consisting of E and S
  VARIANT 26: note = X is selected from the group consisting of K and Q
QDXXGXTPAXXAAXXGXXXIAXVLQXAA

SEQ ID NO: 41 - protein, 28 aa, synthetic construct
  VARIANT 3..26: note = X is selected from any amino acid
QDXXGXTPAXXAAXXGXXXIAXVLQXAA

SEQ ID NO: 42 - protein, 28 aa, synthetic construct
  VARIANT 3..15: note = X is selected from any amino acid
QDXXGXTPADLAAXXGHEDIAEVLQKLN

SEQ ID NO: 43 - protein, 30 aa, synthetic construct
DLGTQLLRAAESGQLDEVRELLKAGADVNA

SEQ ID NO: 44 - protein, 30 aa, synthetic construct
DLGTQLLQAAESGQLDTVRELLKAGADVNA

SEQ ID NO: 45 - protein, 30 aa, synthetic construct
DLGIQLLFAAAKGQLDTVRTLLQAGADVNA

SEQ ID NO: 46 - protein, 33 aa, synthetic construct
KDKDGYTPLHLAAREGHLEIVEVLLSAGADVNA

SEQ ID NO: 47 - protein, 33 aa, synthetic construct
QDEYGLTPLYLATAHGHLEIVTVLLEAGADVNA

SEQ ID NO: 48 - protein, 33 aa, synthetic construct
QDAIGFTPLHLAAFIGHLEIATVLLEAGADVNA

SEQ ID NO: 49 - protein, 33 aa, synthetic construct
QDEYGLTPLYLATAHGHLQIVRVLLQAGADVNA

SEQ ID NO: 50 - protein, 33 aa, synthetic construct
QDAIGFTPLHLAAFIGHLEIATVLLKAGADVNA

SEQ ID NO: 51 - protein, 33 aa, synthetic construct
KDFQGVTPLHIAAQSGHLQIVEVLLQAGADVNA

SEQ ID NO: 52 - protein, 33 aa, synthetic construct
KDVTGDTPLHLAAQHGHLQIVTVLLQAGADVNA

SEQ ID NO: 53 - protein, 28 aa, synthetic construct
QDTSGSTPADLAADAGHEDIAEVLQKAA

SEQ ID NO: 54 - protein, 28 aa, synthetic construct
QDKFGKTPADIAAGAGNEDIAEVLQKAA

SEQ ID NO: 55 - protein, 28 aa, synthetic construct
QDKFGKTPADIAATAGNQQIASVLQKAA

SEQ ID NO: 56 - protein, 28 aa, synthetic construct
QDERGWTPADLAADWGNQQIAEVLQQAA

SEQ ID NO: 57 - protein, 30 aa, synthetic construct
  VARIANT 2..30: note = X is selected from any amino acid
DXXXXXXXXXXXXXXDXXRXXXXXXXDXXX

SEQ ID NO: 58 - skipped

SEQ ID NO: 59 - protein, 30 aa, synthetic construct; identical to SEQ ID NO: 57
  VARIANT 2..14: note = X is selected from any amino acid
  VARIANT 15: note = X is selected from the group consisting of L, V, I, A, M and T
  VARIANT 17..30: note = X is selected from any amino acid

SEQ ID NO: 60-64 - skipped (5 entries)

SEQ ID NO: 65 - protein, 30 aa, synthetic construct
  VARIANT 4..12: note = X is selected from any amino acid
DLGXKLLEAAXXGQDDEVRELLKAGADVNA

SEQ ID NO: 66 - protein, 30 aa, synthetic construct
  VARIANT 4..23: note = X is selected from any amino acid
DLGXKLLEAAXXGQDDXVRXLLXAGADVNA

SEQ ID NO: 67 - protein, 30 aa, synthetic construct
  VARIANT 4..24: note = X is selected from any amino acid
DLGXXLLEAAXXGQDDXVRXLXXXGADVNA

SEQ ID NO: 68 - protein, 30 aa, synthetic construct
  VARIANT 1..24: note = X is selected from any amino acid
XXXXXLLEAAXXGXDXXXXXXXXXGADVNA

SEQ ID NO: 69 - protein, 30 aa, synthetic construct
  VARIANT 1..24: note = X is selected from any amino acid
XXXXXXXEXXXXXXDXXXXXXXXXGADVNA

SEQ ID NO: 70 - protein, 30 aa, synthetic construct
  VARIANT 15: note = X is selected from the group consisting of L, V, I, A, M and T
  VARIANT 1..14: note = X is selected from any amino acid
  VARIANT 16..24: note = X is selected from any amino acid
XXXXXXXEXXXXXXXXXXXXXXXXGADVNA

SEQ ID NO: 71 - protein, 30 aa, synthetic construct
  VARIANT 4..12: note = X is selected from any amino acid
DLGXKLLQAAXXGQLDEVRELLKAGADVNA

SEQ ID NO: 72 - protein, 30 aa, synthetic construct
  VARIANT 4..23: note = X is selected from any amino acid
DLGXKLLQAAXXGQLDXVRXLLXAGADVNA

SEQ ID NO: 73 - protein, 30 aa, synthetic construct
  VARIANT 4..24: note = X is selected from any amino acid
DLGXXLLQAAXXGQLDXVRXLXXXGADVNA

SEQ ID NO: 74 - protein, 30 aa, synthetic construct
  VARIANT 1..24: note = X is selected from any amino acid
XXXXXLLQAAXXGXLXXXXXXXXXGADVNA

SEQ ID NO: 75 - protein, 30 aa, synthetic construct
  VARIANT 1..24: note = X is selected from any amino acid
XXXXXXXQXXXXXXLXXXXXXXXXGADVNA

SEQ ID NO: 76 - protein, 30 aa, synthetic construct
  VARIANT 1..14: note = X is selected from any amino acid
  VARIANT 15: note = X is selected from the group consisting of L, V, I, A, M and T
  VARIANT 16..24: note = X is selected from any amino acid
XXXXXXXQXXXXXXXXXXXXXXXXGADVNA

SEQ ID NO: 77 - protein, 24 aa, synthetic construct
  VARIANT 4..12: note = X is selected from any amino acid
DLGXKLLQAAXXGQLDEVRELLKA

SEQ ID NO: 78 - protein, 24 aa, synthetic construct
  VARIANT 4..23: note = X is selected from any amino acid
DLGXKLLQAAXXGQLDXVRXLLXA

SEQ ID NO: 79 - protein, 24 aa, synthetic construct
  VARIANT 4..24: note = X is selected from any amino acid
DLGXXLLQAAXXGQLDXVRXLXXX

SEQ ID NO: 80-81 - skipped (2 entries)

SEQ ID NO: 82 - protein, 28 aa, synthetic construct
  VARIANT 1..15: note = X is selected from any amino acid
XDXXGXTPADXAADXGHEDIAEVLQKAA

SEQ ID NO: 83 - protein, 28 aa, synthetic construct
  VARIANT 1..26: note = X is selected from any amino acid
XDXXGXTPADXAADXGHEXIAXVLQXAA

SEQ ID NO: 84 - protein, 28 aa, synthetic construct
  VARIANT 1..26: note = X is selected from any amino acid
XDXXGXTPXXXAADXGXEXXXXXXXXAA

SEQ ID NO: 85 - protein, 28 aa, synthetic construct
  VARIANT 1..28: note = X is selected from any amino acid
XDXXGXTPXXXXADXXXEXXXXXXXXXX

SEQ ID NO: 86 - protein, 28 aa, synthetic construct
  VARIANT 1..26: note = X is selected from any amino acid
XDXXGXTPXXXXXDXXXEXXXXXXXXAA

SEQ ID NO: 87 - protein, 28 aa, synthetic construct
  VARIANT 1..15: note = X is selected from any amino acid
XDXXGXTPADXAARXGHQDIAEVLQKAA

SEQ ID NO: 88 - protein, 28 aa, synthetic construct
  VARIANT 1..26: note = X is selected from any amino acid
XDXXGXTPADXAARXGHQXIAXVLQXAA

SEQ ID NO: 89 - protein, 28 aa, synthetic construct
  VARIANT 1..26: note = X is selected from any amino acid
XDXXGXTPXXXAARXGXQXXXXXXXXAA

SEQ ID NO: 90 - protein, 28 aa, synthetic construct
  VARIANT 1..28: note = X is selected from any amino acid
XDXXGXTPXXXXARXXXQXXXXXXXXXX

SEQ ID NO: 91 - protein, 28 aa, synthetic construct
  VARIANT 1..26: note = X is selected from any amino acid
XDXXGXTPXXXXXRXXXQXXXXXXXXAA

SEQ ID NO: 92 - protein, 33 aa, synthetic construct
  VARIANT 1..27: note = X is selected from any amino acid
XDXXGXTPLHXAAXXGHLEIVEVLLKXGADVNA

SEQ ID NO: 93 - protein, 33 aa, synthetic construct
  VARIANT 1..27: note = X is selected from any amino acid
XDXXGXTPLHXAAXXGHPEIVEVLLKXGADVNA

SEQ ID NO: 94 - protein, 33 aa, synthetic construct
  VARIANT 1..27: note = X is selected from any amino acid
XDXXGXTPLHXAXXXGXXXIVXVLLXXGADVNA

SEQ ID NO: 95 - protein, 124 aa, synthetic construct
DLGTQLLQAAESGQLDEVHDLLQAGADVNAQDEYGLTPLYLATAHGHLEIVTVLLEAGADVNAQDAIGFTPLHLAAFIGHLEIATVLLEAGADVNAQDKFGKTPADIAAGAGNEDIAEVLQKAA

SEQ ID NO: 96 - protein, 124 aa, synthetic construct
DLGTQLLEAAESGQLDEVRELLQAGADVNAQDEYGLTPLYLATAHGHLEIVTVLLEAGADVNAQDAIGFTPLHLAAFIGHLEIATVLLEAGADVNAQDKFGKTPADIAAGAGNEDIAEVLQKAA

SEQ ID NO: 97 - protein, 124 aa, synthetic construct
DLGTQLLRAAESGQLDEVRELLKAGADVNAQDEYGLTPLYLATAHGHLEIVTVLLEAGADVNAQDAIGFTPLHLAAFIGHLEIATVLLEAGADVNAQDNFGTTPADIAAGAGNEDIAEVLQQAA

SEQ ID NO: 98 - protein, 124 aa, synthetic construct
DLGTQLLEAAESGQLDEVRELLKAGADVNAQDEYGLTPLYLATAHGHLEIVTVLLEAGADVNAQDAIGFTPLHLAAFIGHLEIATVLLEAGADVNAQDKFGTTPADIAAGAGNEDIAEVLQKAA

SEQ ID NO: 99 - protein, 30 aa, synthetic construct
DLGTQLLQAAESGQLDEVHDLLQAGADVNA

SEQ ID NO: 100 - protein, 30 aa, synthetic construct
DLGTQLLEAAESGQLDEVRELLQAGADVNA

SEQ ID NO: 101 - protein, 30 aa, synthetic construct
DLGTQLLEAAESGQLDEVRELLKAGADVNA

SEQ ID NO: 102 - protein, 28 aa, synthetic construct
QDNFGTTPADIAAGAGNEDIAEVLQQAA

SEQ ID NO: 103 - protein, 28 aa, synthetic construct
QDKFGTTPADIAAGAGNEDIAEVLQKAA

SEQ ID NO: 104 - protein, 124 aa, synthetic construct
  VARIANT 4..111: note = X is selected from any amino acid
DLGXQLLXAAXXGQLDTVRELLAAGADVNASDXXGXTPLHXAAXXGHLEIVEVLLEAGADVNASDXXGXTPLHXAAXXGHLEIVEVLLEAGADVNAQDXXGXTPADLAAXXGNEDIAEVLQKAA

SEQ ID NO: 105 - protein, 124 aa, synthetic construct
  VARIANT 4..111: note = X is selected from any amino acid
DLGXKLLXAAXXGQLDEVRELLKAGADVNAKDXXGXTPLHXAAXXGHLEIVEVLLEAGADVNAKDXXGXTPLHXAAXXGHLEIVEVLLEAGADVNAQDXXGXTPADLAAXXGHEDIAEVLQQAA

SEQ ID NO: 106 - protein, 124 aa, synthetic construct
  VARIANT 4..111: note = X is selected from any amino acid
DLGXKLLXAAXXGQLDEVRELLKAGADVNAKDXXGXTPLHXAAXXGHLEIVEVLLSAGADVNAKDXXGXTPLHXAAXXGHLEIVEVLLSAGADVNAQDXXGXTPADLAAXXGHEDIAEVLQKAA

SEQ ID NO: 107 - protein, 124 aa, synthetic construct
  VARIANT 4..111: note = X is selected from any amino acid
DLGXQLLXAAXXGQLDEVRELLAAGADVNAKDXXGXTPLHXAAXXGNLEIVEVLLSAGADVNAKDXXGXTPLHXAAXXGNLEIVEVLLSAGADVNAQDXXGXTPADLAAXXGHEDIAEVLQKAA

SEQ ID NO: 108 - protein, 124 aa, synthetic construct
  VARIANT 4..111: note = X is selected from any amino acid
DLGXQLLXAAXXGQLDEVRELLKAGADVNAQDXXGXTPLYXATXXGHLEIVTVLLEAGADVNAQDXXGXTPLHXAAXXGHLEIATVLLEAGADVNAQDXXGXTPADIAAXXGNEDIAEVLQKAA

SEQ ID NO: 109 - protein, 124 aa, synthetic construct
  VARIANT 4..111: note = X is selected from any amino acid
DLGXQLLXAAXXGQLDTVRTLLKAGADVNAQDXXGXTPLYXATXXGHLQIVQVLLSAGADVNAQDXXGXTPLHXAAXXGHLQIAQVLLSAGADVNAQDXXGXTPAQIAAXXGNQQIASVLQQAA

SEQ ID NO: 110 - protein, 124 aa, synthetic construct
  VARIANT 4..111: note = X is selected from any amino acid
DLGXQLLXAAXXGQLDTVRELLKAGADVNAQDXXGXTPLYXATXXGHLQIVRVLLQAGADVNAQDXXGXTPLHXAAXXGHLEIATVLLKAGADVNAQDXXGXTPADIAAXXGNQQIASVLQKAA

SEQ ID NO: 111 - protein, 124 aa, synthetic construct
  VARIANT 4..111: note = X is selected from any amino acid
DLGXQLLXAAXXGQLDTVRTLLQAGADVNAKDXXGXTPLHXAAXXGHLQIVEVLLQAGADVNAKDXXGXTPLHXAAXXGHLQIVTVLLQAGADVNAQDXXGXTPADLAAXXGNQQIAEVLQQAA

SEQ ID NO: 112 - protein, 124 aa, synthetic construct
  VARIANT 4..111: note = X is selected from any amino acid
DLGXQLLXAAXXGQLDEVHDLLQAGADVNAQDXXGXTPLYXATXXGHLEIVTVLLEAGADVNAQDXXGXTPLHXAAXXGHLEIATVLLEAGADVNAQDXXGXTPADIAAXXGNEDIAEVLQKAA

SEQ ID NO: 113 - protein, 124 aa, synthetic construct
  VARIANT 4..111: note = X is selected from any amino acid
DLGXQLLXAAXXGQLDEVRELLQAGADVNAQDXXGXTPLYXATXXGHLEIVTVLLEAGADVNAQDXXGXTPLHXAAXXGHLEIATVLLEAGADVNAQDXXGXTPADIAAXXGNEDIAEVLQKAA

SEQ ID NO: 114 - protein, 124 aa, synthetic construct
  VARIANT 4..111: note = X is selected from any amino acid
DLGXQLLXAAXXGQLDEVRELLKAGADVNAQDXXGXTPLYXATXXGHLEIVTVLLEAGADVNAQDXXGXTPLHXAAXXGHLEIATVLLEAGADVNAQDXXGXTPADIAAXXGNEDIAEVLQQAA

SEQ ID NO: 115 - protein, 30 aa, synthetic construct
  VARIANT 4..12: note = X is selected from any amino acid
DLGXQLLXAAXXGQLDTVRELLAAGADVNA

SEQ ID NO: 116 - protein, 30 aa, synthetic construct
  VARIANT 4..12: note = X is selected from any amino acid
DLGXQLLXAAXXGQLDEVRELLAAGADVNA

SEQ ID NO: 117 - protein, 30 aa, synthetic construct
  VARIANT 4..12: note = X is selected from any amino acid
DLGXQLLXAAXXGQLDEVRELLKAGADVNA

SEQ ID NO: 118 - protein, 30 aa, synthetic construct
  VARIANT 4..12: note = X is selected from any amino acid
DLGXQLLXAAXXGQLDTVRTLLKAGADVNA

SEQ ID NO: 119 - protein, 30 aa, synthetic construct
  VARIANT 4..12: note = X is selected from any amino acid
DLGXQLLXAAXXGQLDTVRELLKAGADVNA

SEQ ID NO: 120 - protein, 30 aa, synthetic construct
  VARIANT 4..12: note = X is selected from any amino acid
DLGXQLLXAAXXGQLDTVRTLLQAGADVNA

SEQ ID NO: 121 - protein, 30 aa, synthetic construct
  VARIANT 4..12: note = X is selected from any amino acid
DLGXQLLXAAXXGQLDEVHDLLQAGADVNA

SEQ ID NO: 122 - protein, 30 aa, synthetic construct
  VARIANT 4..12: note = X is selected from any amino acid
DLGXQLLXAAXXGQLDEVRELLQAGADVNA

SEQ ID NO: 123 - protein, 28 aa, synthetic construct
  VARIANT 3..15: note = X is selected from any amino acid
QDXXGXTPADLAAXXGNEDIAEVLQKAA

SEQ ID NO: 124 - protein, 28 aa, synthetic construct
  VARIANT 3..15: note = X is selected from any amino acid
QDXXGXTPADLAAXXGHEDIAEVLQQAA

SEQ ID NO: 125 - protein, 28 aa, synthetic construct
  VARIANT 3..15: note = X is selected from any amino acid
QDXXGXTPADLAAXXGHEDIAEVLQKAA

SEQ ID NO: 126 - protein, 28 aa, synthetic construct
  VARIANT 3..15: note = X is selected from any amino acid
QDXXGXTPADIAAXXGNEDIAEVLQKAA

SEQ ID NO: 127 - protein, 28 aa, synthetic construct
  VARIANT 3..15: note = X is selected from any amino acid
QDXXGXTPAQIAAXXGNQQIASVLQQAA

SEQ ID NO: 128 - protein, 28 aa, synthetic construct
  VARIANT 3..15: note = X is selected from any amino acid
QDXXGXTPADIAAXXGNQQIASVLQKAA

SEQ ID NO: 129 - protein, 28 aa, synthetic construct
  VARIANT 3..15: note = X is selected from any amino acid
QDXXGXTPADLAAXXGNQQIAEVLQQAA

SEQ ID NO: 130 - protein, 28 aa, synthetic construct
  VARIANT 3..15: note = X is selected from any amino acid
QDXXGXTPADIAAXXGNEDIAEVLQQAA

SEQ ID NO: 131 - protein, 33 aa, synthetic construct
  VARIANT 3..15: note = X is selected from any amino acid
SDXXGXTPLHXAAXXGHLEIVEVLLEAGADVNA

SEQ ID NO: 132 - protein, 33 aa, synthetic construct
  VARIANT 3..15: note = X is selected from any amino acid
KDXXGXTPLHXAAXXGHLEIVEVLLEAGADVNA

SEQ ID NO: 133 - protein, 33 aa, synthetic construct
  VARIANT 3..15: note = X is selected from any amino acid
KDXXGXTPLHXAAXXGHLEIVEVLLSAGADVNA

SEQ ID NO: 134 - protein, 33 aa, synthetic construct
  VARIANT 3..15: note = X is selected from any amino acid
KDXXGXTPLHXAAXXGNLEIVEVLLSAGADVNA

SEQ ID NO: 135 - protein, 33 aa, synthetic construct
  VARIANT 3..15: note = X is selected from any amino acid
QDXXGXTPLYXATXXGHLEIVTVLLEAGADVNA

SEQ ID NO: 136 - protein, 33 aa, synthetic construct
  VARIANT 3..15: note = X is selected from any amino acid
QDXXGXTPLYXATXXGHLQIVQVLLSAGADVNA

SEQ ID NO: 137 - protein, 33 aa, synthetic construct
  VARIANT 3..15: note = X is selected from any amino acid
QDXXGXTPLYXATXXGHLQIVRVLLQAGADVNA

SEQ ID NO: 138 - protein, 33 aa, synthetic construct
  VARIANT 3..15: note = X is selected from any amino acid
KDXXGXTPLHXAAXXGHLQIVEVLLQAGADVNA

SEQ ID NO: 139 - protein, 33 aa, synthetic construct
  VARIANT 3..15: note = X is selected from any amino acid
QDXXGXTPLHXAAXXGHLEIATVLLEAGADVNA

SEQ ID NO: 140 - protein, 33 aa, synthetic construct
  VARIANT 3..15: note = X is selected from any amino acid
QDXXGXTPLHXAAXXGHLQIAQVLLSAGADVNA

SEQ ID NO: 141 - protein, 33 aa, synthetic construct
  VARIANT 3..15: note = X is selected from any amino acid
QDXXGXTPLHXAAXXGHLEIATVLLKAGADVNA

SEQ ID NO: 142 - protein, 33 aa, synthetic construct
  VARIANT 3..15: note = X is selected from any amino acid
KDXXGXTPLHXAAXXGHLQIVTVLLQAGADVNA